SEQUENCE LISTING (44 sequences)

SEQ ID NO: 1 - DNA, 807 bp, Burkholderia fungorum LB400
  CDS (1)..(807): Coding sequence for Burkholderia fungorum LB400 RBU11704 xylose dehydrogenase
atgtatttgttgtcatacccggaacaggtggactatccgatgtcgtacgcaatctatcccagcctctcaggcaaaacggttgtcatcaccggcggcggcagcggcatcggcgccgcgatggtcgaagctttcgcccggcagggcgcgcgagttttcttcctcgacgtcgctgaggacgattcgctggcgttgcagcaatcgctgagcgacgcgcctcacccgccgttgttccgccgctgcgatctgcgcagcgtcgatgcgatccacagtgcgtttgccgggatcgtcgagatcgccgggccgatcgaggtactcgtcaacaacgctggcaacgacgaccggcatgaagtcgacgccatcacgccggcctattgggacgagcgcatggccgtgaacctgcggcaccagttcttctgcgcgcaggccgcagcggccggcatgcgcaagatcgggcgcggcgtgatcctgaatcttggctcggtttcctggcacctcgcgttgccgaacctcgcgatctacatgagcgcgaaggccggtatcgaagggctgacccggggcctcgcgcgcgatctcggcgccgccggcatccgcgtgaactgcattattcccggcgcggtgcggactccccgtcagatgcagctctggcagtcgcccgagagcgaagcgaagctcgtcgccagccaatgtctgcgtttgcgtatcgaacctgagcatgtcgcgcgcatggcgttgtttcttgcgtccgacgatgcgtcgcgttgctcagggcgggattatttcgtcgacgccgggtggtacggagaatga

SEQ ID NO: 2 - protein, 268 aa, Burkholderia fungorum LB400
MYLLSYPEQVDYPMSYAIYPSLSGKTVVITGGGSGIGAAMVEAFARQGARVFFLDVAEDDSLALQQSLSDAPHPPLFRRCDLRSVDAIHSAFAGIVEIAGPIEVLVNNAGNDDRHEVDAITPAYWDERMAVNLRHQFFCAQAAAAGMRKIGRGVILNLGSVSWHLALPNLAIYMSAKAGIEGLTRGLARDLGAAGIRVNCIIPGAVRTPRQMQLWQSPESEAKLVASQCLRLRIEPEHVARMALFLASDDASRCSGRDYFVDAGWYGE

SEQ ID NO: 3 - DNA, 747 bp, Caulobacter crescentus CB15
  CDS (1)..(747): Coding sequence for Caulobacter crescentus CB15 RCO01012 xylose dehydrogenase
atgtcctcagccatctatcccagcctgaagggcaagcgcgtcgtcatcaccggcggcggctcgggcatcggggccggcctcaccgccggcttcgcccgtcagggcgcggaggtgatcttcctcgacatcgccgacgaggactccagggctcttgaggccgagctggccggctcgccgatcccgccggtctacaagcgctgcgacctgatgaacctcgaggcgatcaaggcggtcttcgccgagatcggcgacgtcgacgtgctggtcaacaacgccggcaatgacgaccgccacaagctggccgacgtgaccggcgcctattgggacgagcggatcaacgtcaacctgcgccacatgctgttctgcacccaggccgtcgcgccgggcatgaagaagcgtggcggcggggcggtgatcaacttcggttcgatcagctggcacctggggcttgaggacctcgtcctctacgaaaccgccaaggccggcatcgaaggcatgacccgcgcgctggcccgggagctgggtcccgacgacatccgcgtcacctgcgtggtgccgggcaacgtcaagaccaagcgccaggagaagtggtacacgcccgaaggcgaggcccagatcgtggcggcccaatgcctgaagggccgcatcgtcccggagaacgtcgccgcgctggtgctgttcctggcctcggatgacgcgtcgctctgcaccggccacgaatactggatcgacgccggctggcgttga

SEQ ID NO: 4 - protein, 248 aa, Caulobacter crescentus CB15
MSSAIYPSLKGKRVVITGGGSGIGAGLTAGFARQGAEVIFLDIADEDSRALEAELAGSPIPPVYKRCDLMNLEAIKAVFAEIGDVDVLVNNAGNDDRHKLADVTGAYWDERINVNLRHMLFCTQAVAPGMKKRGGGAVINFGSISWHLGLEDLVLYETAKAGIEGMTRALARELGPDDIRVTCVVPGNVKTKRQEKWYTPEGEAQIVAAQCLKGRIVPENVAALVLFLASDDASLCTGHEYWIDAGWR

SEQ ID NO: 5 - DNA, 1968 bp, Escherichia coli yjhG
  CDS (1)..(1968): Coding sequence for E. coli yjhG  xylonate dehydratase
atgtctgttcgcaatatttttgctgacgagagccacgatatttacaccgtcagaacgcacgccgatggcccggacggcgaactcccattaaccgcagagatgcttatcaaccgcccgagcggggatctgttcggtatgaccatgaatgccggaatgggttggtctccggacgagctggatcgggacggtattttactgctcagtacactcggtggcttacgcggcgcagacggtaaacccgtggcgctggcgttgcaccaggggcattacgaactggacatccagatgaaagcggcggccgaggttattaaagccaaccatgccctgccctatgccgtgtacgtctccgatccttgtgacgggcgtactcagggtacaacggggatgtttgattcgctaccataccgaaatgacgcatcgatggtaatgcgccgccttattcgctctctgcccgacgcgaaagcagttattggtgtggcgagttgcgataaggggcttccggccaccatgatggcactcgccgcgcagcacaacatcgcaaccgtgctggtccccggcggcgcgacgctgcccgcaaaggatggagaagacaacggcaaggtgcaaaccattggcgcacgcttcgccaatggcgaattatctctacaggacgcacgccgtgcgggctgtaaagcctgtgcctcttccggcggcggctgtcaatttttgggcactgccgggacatctcaggtggtggccgaaggattgggactggcaatcccacattcagccctggccccttccggtgagcctgtgtggcgggagatcgccagagcttccgcgcgagctgcgctgaacctgagtcaaaaaggcatcaccacccgggaaattctcaccgataaagcgatagagaatgcgatgacggtccatgccgcgttcggtggttcaacaaacctgctgttacacatcccggcaattgctcaccaggcaggttgccatatcccgaccgttgatgactggatccgcatcaacaagcgcgtgccccgactggtgagcgtactgcctaatggcccggtttatcatccaacggtcaatgcctttatggcaggtggtgtgccggaagtcatgttgcatctgcgcagcctcggattgttgcatgaagacgttatgacggttaccggcagcacgctgaaagaaaacctcgactggtgggagcactccgaacggcgtcagcggttcaagcaactcctgctcgatcaggaacaaatcaacgctgacgaagtgatcatgtctccgcagcaagcaaaagcgcgcggattaacctcaactatcaccttcccggtgggcaatattgcgccagaaggttcggtgatcaaatccaccgccattgacccctcgatgattgatgagcaaggtatctattaccataaaggtgtggcgaaggtttatctgtccgagaaaagtgcgatttacgatatcaaacatgacaagatcaaggcgggcgatattctggtcattattggcgttggaccttcaggtacagggatggaagaaacctaccaggttaccagtgccctgaagcatctgtcatacggtaagcatgtttcgttaatcaccgatgcacgtttctcgggcgtttctactggcgcgtgcatcggccatgtggggccagaagcgctggccggaggccccatcggtaaattacgcaccggggatttaattgaaattaaaattgattgtcgcgagcttcacggcgaagtcaatttcctcggaacccgtagcgatgaacaattaccttcacaggaggaggcaactgcaatattaaatgccagacccagccatcaggatttacttcccgatcctgaattgccagatgatacccggctatgggcaatgcttcaggccgtgagtggtgggacatggaccggttgtatttatgatgtaaacaaaattggcgcggctttgcgcgattttatgaataaaaactga

SEQ ID NO: 6 - protein, 655 aa, Escherichia coli yjhG [sequence omitted]

SEQ ID NO: 7 - DNA, 1968 bp, Escherichia coli yagF
  CDS (1)..(1968): Coding sequence for Escherichia coli yagF xylonate dehydratase
atgaccattgagaaaattttcaccccgcaggacgacgcgttttatgcggtgatcacccacgcggcggggccgcagggcgctctgccgctgaccccgcagatgctgatggaatctcccagcggcaacctgttcggcatgacgcagaacgccgggatgggctgggacgccaacaagctcaccggcaaagaggtgctgattatcggcactcagggcggcatccgcgccggagacggacgcccaatcgcgctgggctaccacaccgggcattgggagatcggcatgcagatgcaggcggcggcgaaggagatcacccgcaatggcgggatcccgttcgcggccttcgtcagcgatccgtgcgacgggcgctcgcagggcacgcacggtatgttcgattccctgccgtaccgcaacgacgcggcgatcgtgtttcgccgcctgatccgctccctgccgacgcggcgggcggtgatcggcgtagcgacctgcgataaagggctgcccgccaccatgattgcgctggccgcgatgcacgacctgccgactattctggtgccgggcggggcgacgctgccgccgaccgtcggggaagacgcgggcaaggtgcagaccatcggcgcgcgtttcgccaaccacgaactctccctgcaggaggccgccgaactgggctgtcgcgcctgcgcctcgccgggcggcgggtgtcagttcctcggcacggcgggcacctcgcaggtggtcgcggaggcgctgggtctggcgctgccgcactccgcgctggcgccgtccgggcaggcggtgtggctggagatcgcccgccagtcggcgcgcgcggtcagcgagctggatagccgcggcatcaccacgcgggatatcctctccgataaagccatcgaaaacgcgatggtgatccacgcggcgttcggcggctccaccaatttactgctgcacattccggccatcgcccacgcggcgggctgcacgatcccggacgttgagcactggacgcgcatcaaccgtaaagtgccgcgtctggtgagcgtgctgcccaacggcccggactatcacccgaccgtgcgcgccttcctcgcgggcggcgtgccggaggtgatgctccacctgcgcgacctcggcctgctgcatctggacgccatgaccgtgaccggccagacggtgggcgagaaccttgaatggtggcaggcgtccgagcgccgggcgcgcttccgccagtgcctgcgcgagcaggacggcgtagagccggatgacgtgatcctgccgccggagaaggcaaaagcgaaagggctgacctcgacggtctgcttcccgacgggcaacatcgctccggaaggttcggtgatcaaggccacggcgatcgacccgtcggtggtgggcgaagatggcgtataccaccacaccggccgggtgcgggtgtttgtctcggaagcgcaggcgatcaaggcgatcaagcgggaagagattgtgcagggcgatatcatggtggtgatcggcggcgggccgtccggcaccggcatggaagagacctaccagctcacctccgcgctaaagcatatctcgtggggcaagacggtgtcgctcatcaccgatgcgcgcttctcgggcgtgtcgacgggcgcctgcttcggccacgtgtcgccggaggcgctggcgggcgggccgattggcaagctgcgcgataacgacatcatcgagattgccgtggatcgtctgacgttaactggcagcgtgaacttcatcggcaccgcggacaacccgctgacgccggaagagggcgcgcgcgagctggcgcggcggcagacgcacccggacctgcacgcccacgactttttgccggacgacacccggctgtgggcggcactgcagtcggtgagcggcggcacctggaaaggctgtatttatgacaccgataaaattatcgaggtaattaacgccggtaaaaaagcgctcggaatttaa

SEQ ID NO: 8 - protein, 655 aa, Escherichia coli yagF
MTIEKIFTPQDDAFYAVITHAAGPQGALPLTPQMLMESPSGNLFGMTQNAGMGWDANKLTGKEVLIIGTQGGIRAGDGRPIALGYHTGHWEIGMQMQAAAKEITRNGGIPFAAFVSDPCDGRSQGTHGMFDSLPYRNDAAIVFRRLIRSLPTRRAVIGVATCDKGLPATMIALAAMHDLPTILVPGGATLPPTVGEDAGKVQTIGARFANHELSLQEAAELGCRACASPGGGCQFLGTAGTSQVVAEALGLALPHSALAPSGQAVWLEIARQSARAVSELDSRGITTRDILSDKAIENAMVIHAAFGGSTNLLLHIPAIAHAAGCTIPDVEHWTRINRKVPRLVSVLPNGPDYHPTVRAFLAGGVPEVMLHLRDLGLLHLDAMTVTGQTVGENLEWWQASERRARFRQCLREQDGVEPDDVILPPEKAKAKGLTSTVCFPTGNIAPEGSVIKATAIDPSVVGEDGVYHHTGRVRVFVSEAQAIKAIKREEIVQGDIMVVIGGGPSGTGMEETYQLTSALKHISWGKTVSLITDARFSGVSTGACFGHVSPEALAGGPIGKLRDNDIIEIAVDRLTLTGSVNFIGTADNPLTPEEGARELARRQTHPDLHAHDFLPDDTRLWAALQSVSGGTWKGCIYDTDKIIEVINAGKKALGI

SEQ ID NO: 9 - DNA, 411 bp, Pseudomonas fragi
  CDS (1)..(411): Coding sequence for Pseudomonas fragi ATCC 4973 xylonate dehydratase fragment.
ctcgaggattggcagcgcgtgggtgaagacgtgcccttgctggtcaactgcatgcctgccggcgagtacctgggcgaaagcttccaccgcgccggtggcgtaccggcggtgatgcatgagctggacaaagtgggccgcctgcaccgcgattgcctcacggtcagtggccgcaacatgggtgaagtggtcgccgactgcgtcaccggcgaccgcgacgtgatccgctcctacgaagacccgctgatgcaccgcgctggttttattgtgctcagcggcaacttcttcgacagcgcgatcatgaaaatgtcggtggtgggcgaagccttccgcaagacctacctcagcgacccgctgcaacccaacagcttcgaggcgcgggccattgtgttcgaaggccccgaagactacacn

SEQ ID NO: 10 - protein, 137 aa, Pseudomonas fragi
LEDWQRVGEDVPLLVNCMPAGEYLGESFHRAGGVPAVMHELDKVGRLHRDCLTVSGRNMGEVVADCVTGDRDVIRSYEDPLMHRAGFIVLSGNFFDSAIMKMSVVGEAFRKTYLSDPLQPNSFEARAIVFEGPEDYT

SEQ ID NO: 11 - DNA, 960 bp, Escherichia coli
  misc_feature (1)..(960): Coding sequence for E. coli yjhH 3-deoxy-D-glycero-pentulosonate aldolase
atgggctgggatacagaaacgaaaatgagcacttacgaaaaggaaactgaggtaatgaaaaaattcagcggcattattccaccggtatccagcacgtttcatcgtgacggaacccttgataaaaaggcaatgcgcgaagttgccgacttcctgattaataaaggggtcgacgggctgttttatctgggtaccggtggtgaatttagccaaatgaatacagcccagcgcatggcactcgccgaagaagctgtaaccattgtcgacgggcgagtgccggtattgattggcgtcggttccccttccactgacgaagcggtcaaactggcgcagcatgcgcaagcctacggcgctgatggtatcgtcgccatcaacccctactactggaaagtcgcaccacgaaatcttgacgactattaccagcagatcgcccgtagcgtcaccctaccggtgatcctgtacaactttccggatctgacgggtcaggacttaaccccggaaaccgtgacgcgtctggctctgcaaaacgagaatatcgttggcatcaaagacaccatcgacagcgttggtcacttgcgtacgatgatcaacacagttaagtcggtacgcccgtcgttttcggtattctgcggttacgatgatcatttgctgaatacgatgctgctgggcggcgacggtgcgataaccgccagcgctaactttgctccggaactctccgtcggcatctaccgcgcctggcgtgaaggcgatctggcgaccgctgcgacgctgaataaaaaactactacaactgcccgctatttacgccctcgaaacaccgtttgtctcactgatcaaatacagcatgcagtgtgtcgggctgcctgtagagacatattgcttaccaccgattcttgaagcatctgaagaagcaaaagataaagtccacgtgctgcttaccgcgcagggcattttaccagtctga

SEQ ID NO: 12 - protein, 319 aa, Escherichia coli
  SITE (1)..(1): Putative initiator Met
MGWDTETKMSTYEKETEVMKKFSGIIPPVSSTFHRDGTLDKKAMREVADFLINKGVDGLFYLGTGGEFSQMNTAQRMALAEEAVTIVDGRVPVLIGVGSPSTDEAVKLAQHAQAYGADGIVAINPYYWKVAPRNLDDYYQQIARSVTLPVILYNFPDLTGQDLTPETVTRLALQNENIVGIKDTIDSVGHLRTMINTVKSVRPSFSVFCGYDDHLLNTMLLGGDGAITASANFAPELSVGIYRAWREGDLATAATLNKKLLQLPAIYALETPFVSLIKYSMQCVGLPVETYCLPPILEASEEAKDKVHVLLTAQGILPV

SEQ ID NO: 13 - DNA, 930 bp, Escherichia coli
  CDS (1)..(930): Coding sequence for E. coli yagE 3-deoxy-D-glycero-pentulosonate aldolase
atgattcagcaaggagatctcatgccgcagtccgcgttgttcacgggaatcattccccctgtctccaccatttttaccgccgacggccagctcgataagccgggcaccgccgcgctgatcgacgatctgatcaaagcaggcgttgacggcctgttcttcctgggcagcggtggcgagttctcccagctcggcgccgaagagcgtaaagccattgcccgctttgctatcgatcatgtcgatcgtcgcgtgccggtgctgatcggcaccggcggcaccaacgcccgggaaaccatcgaactcagccagcacgcgcagcaggcgggcgcggacggcatcgtggtgatcaacccctactactggaaagtgtcggaagcgaacctgatccgctatttcgagcaggtggccgacagcgtcacgctgccggtgatgctctataacttcccggcgctgaccgggcaggatctgactccggcgctggtgaaaaccctcgccgactcgcgcagcaatattatcggcatcaaagacaccatcgactccgtcgcccacctgcgcagcatgatccataccgtcaaaggtgcccatccgcacttcaccgtgctctgcggctacgacgatcatctgttcaataccctgctgctcggcggcgacggggcgatatcggcgagcggcaactttgccccgcaggtgtcggtgaatcttctgaaagcctggcgcgacggggacgtggcgaaagcggccgggtatcatcagaccttgctgcaaattccgcagatgtatcagctggatacgccgtttgtgaacgtgattaaagaggcgatcgtgctctgcggtcgtcctgtctccacgcacgtgctgccgcccgcctcgccgctggacgagccgcgcaaggcgcagctgaaaaccctgctgcaacagctcaagctttgctga

SEQ ID NO: 14 - protein, 309 aa, Escherichia coli
MIQQGDLMPQSALFTGIIPPVSTIFTADGQLDKPGTAALIDDLIKAGVDGLFFLGSGGEFSQLGAEERKAIARFAIDHVDRRVPVLIGTGGTNARETIELSQHAQQAGADGIVVINPYYWKVSEANLIRYFEQVADSVTLPVMLYNFPALTGQDLTPALVKTLADSRSNIIGIKDTIDSVAHLRSMIHTVKGAHPHFTVLCGYDDHLFNTLLLGGDGAISASGNFAPQVSVNLLKAWRDGDVAKAAGYHQTLLQIPQMYQLDTPFVNVIKEAIVLCGRPVSTHVLPPASPLDEPRKAQLKTLLQQLKLC

SEQ ID NO: 15 - DNA, 28 bp, Artificial Sequence
  Forward amplification primer for Burkholderia fungorum LB400 D-xylose dehydrogenase gene (RBU11704)
cgggatccatgtatttgttgtcataccc

SEQ ID NO: 16 - DNA, 27 bp, Artificial Sequence
  Reverse amplification primer for B. fungorum LB400 D-xylose dehydrogenase gene (RBU11704)
cgggatccatatcgacgaaataaaccg

SEQ ID NO: 17 - DNA, 28 bp, Artificial Sequence
  Forward amplification primer for Caulobacter crescentus CB15 D-xylose dehydrogenase gene (RCO01012)
gcggatccatgtcctcagccatctatcc

SEQ ID NO: 18 - DNA, 28 bp, Artificial Sequence
  Reverse amplification primer for C. crescentus CB15 D-xylose dehydrogenase gene (RCO01012)
gcggatccgatgacagttttcttaggtc

SEQ ID NO: 19 - DNA, 26 bp, Artificial Sequence
  Forward amplification primer for E. coli W3110 D-xylonate dehydratase gene (yjhG)
cggaattcatgtctgttcgcaatatt

SEQ ID NO: 20 - DNA, 26 bp, Artificial Sequence
  Reverse amplification primer for E. coli W3110 D-xylonate dehydratase gene (yjhG)
gcaagcttaattcaggtgtctggatg

SEQ ID NO: 21 - DNA, 26 bp, Artificial Sequence
  Forward amplification primer for E. coli W3110 D-xylonate dehydratase gene (yagF)
cggaattcgatgaccattgagaaaat

SEQ ID NO: 22 - DNA, 26 bp, Artificial Sequence
  Reverse amplification primer for E. coli W3110 D-xylonate dehydratase gene (yagF)
gcaagcttcaacgatatatctcaact

SEQ ID NO: 23 - DNA, 28 bp, Artificial Sequence
  Forward amplification primer for E. coli W3110 3-deoxy-D-glycero-pentulosonate aldolase gene (yjhH)
cggaattcatgggctgggatacagaaac

SEQ ID NO: 24 - DNA, 28 bp, Artificial Sequence
  Reverse amplification primer for E. coli W3110 3-deoxy-D-glycero-pentulosonate aldolase gene (yjhH)
gcggatcctcagactggtaaaatgccct

SEQ ID NO: 25 - DNA, 27 bp, Artificial Sequence
  Forward amplification primer for E. coli W3110 3-deoxy-D-glycero-pentulosonate aldolase gene (yagE)
cggaattcatgattcagcaaggagatc

SEQ ID NO: 26 - DNA, 27 bp, Artificial Sequence
  Reverse amplification primer for E. coli W3110 3-deoxy-D-glycero-pentulosonate aldolase gene (yagE)
taggatccttatcgtccggctcagcaa

SEQ ID NO: 27 - DNA, 28 bp, Artificial Sequence
  Forward amplification primer for C. crescentus CB15 D-xylose dehydrogenase gene, for construction of plasmid pWN9.068A
gcgcatgcatgtcctcagccatctatcc

SEQ ID NO: 28 - DNA, 28 bp, Artificial Sequence
  Reverse amplification primer for C. crescentus CB15 D-xylose dehydrogenase gene, for construction of plasmid pWN9.068A
gcgcatgcgatgacagttttcttaggtc

SEQ ID NO: 29 - DNA, 20 bp, Artificial Sequence
  Forward amplification primer for Pseudomonas fragi xylonate dehydratase gene
ctggargaytggcarcgygt

SEQ ID NO: 30 - DNA, 20 bp, Artificial Sequence
  Reverse amplification primer for Pseudomonas fragi xylonate dehydratase gene
gtrtartcytcrggrccytc

SEQ ID NO: 31 - DNA, 61 bp, Artificial Sequence
  Forward amplification primer for the DNA fragment for use in disrupting the E. coli genomic 3-deoxy-D-glycero-pentulosonate aldolase gene (yjhH)
gttgccgacttcctgattaataaaggggtcgacgggctgtgtgtaggctggagctgcttcg

SEQ ID NO: 32 - DNA, 61 bp, Artificial Sequence
  Reverse amplification primer for the DNA fragment for use in disrupting the E. coli genomic 3-deoxy-D-glycero-pentulosonate aldolase gene (yjhH)
aactgtgttgatcatcgtacgcaagtgaccaacgctgtcgcatatgaatatcctccttagt

SEQ ID NO: 33 - DNA, 57 bp, Artificial Sequence
  Forward amplification primer for the DNA fragment for use in disrupting the E. coli genomic 3-deoxy-D-glycero-pentulosonate aldolase gene (yagE)
ccgggaaaccatcgaactcagccagcacgcgcagcacatatgaatatcctccttagt

SEQ ID NO: 34 - DNA, 57 bp, Artificial Sequence
  Reverse amplification primer for the DNA fragment for use in disrupting the E. coli genomic 3-deoxy-D-glycero-pentulosonate aldolase gene (yagE)
ggatgggcacctttgacggtatggatcatgctgcgcgtgtaggctggagctgcttcg

SEQ ID NO: 35 - DNA, 57 bp, Artificial Sequence
  Forward amplification primer for the DNA fragment for use in inserting xdh into the E. coli genomic DNA
tacgacatcatccatcacccgcggcattacctgattatgtcctcagccatctatccc

SEQ ID NO: 36 - DNA, 55 bp, Artificial Sequence
  Reverse amplification primer for the DNA fragment for use in inserting xdh into the E. coli genomic DNA
cagaagttgctgatagaggcgacggaacgtttctcatatgaatatcctccttagt

SEQ ID NO: 37 - DNA, 1011 bp, Escherichia coli
  misc_feature (1)..(1011): Coding Sequence for E. coli AdhP alcohol dehydrogenase, from GenBank U00096
ttagtgacggaaatcaatcaccatgcggccacggattttgccttcttccatctcagtaaagatggtgttgatgtccgctaacggacgcagggcgactttcggcaccactttaccttcggcggcaaactggaaggcttcagttaaatcctggcgcgtgccgaccagcgaaccgaccacttcaataccatccagcacaagacgtgggatatccaggctcatagactccggcggtagaccgacagccacaacacgaccgcctgcacggacagcatcaactgccgagttaaacgcagctttagctaccgctgttaccaccgcagcgtgagcgccaccagttttctcctgcacaattttggcggcgtcttcggtgtgtgagttaatcgctaaatctgcgcccatttcggttgccagttttaactgctcatcattgacatcaatggcgatcactttggcgttaaagacattcttcgcgtattgcagggcgaggttacccagaccgccaagaccgtagatagcaatccactgccctggacgaatttttgacagcttaacggctttgtaggtggtgactcccgcacaggtaatgctgctggccgccgccgagtccagaccatctggcacttttaccgcgtaatcggcgaccacgatgcactcttccgccatcccgccatcaacgctgtatccggcatttttaactgaacggcagagcgtttcgttaccactgttacagtattcgcaatgaccgcatccttcgtagaaccacgccacgctggcacgatcgcctggttttaatgaggtgacacctggacccacttctgccaccacaccgatgccttcatggcccagaattacgccggttttgtcaccaaaatcgccattcttaacatgaagatcggtatgacatacaccacaacactccattttcagcagggcttcgccatgtttcagtgagcgcagtgttttatacgtaacgtcaacatgatgatccttcgtaacaactgcagccttcat

SEQ ID NO: 38 - protein, 336 aa, Escherichia coli
  MISC_FEATURE (1)..(336): AdhP 1-propanol-preferring, two-zinc-ion-containing alcohol dehydrogenase (Genbank Accession No. AAC74551) of IUBMB EC 1.1.1.1
MKAAVVTKDHHVDVTYKTLRSLKHGEALLKMECCGVCHTDLHVKNGDFGDKTGVILGHEGIGVVAEVGPGVTSLKPGDRASVAWFYEGCGHCEYCNSGNETLCRSVKNAGYSVDGGMAEECIVVADYAVKVPDGLDSAAASSITCAGVTTYKAVKLSKIRPGQWIAIYGLGGLGNLALQYAKNVFNAKVIAIDVNDEQLKLATEMGADLAINSHTEDAAKIVQEKTGGAHAAVVTAVAKAAFNSAVDAVRAGGRVVAVGLPPESMSLDIPRLVLDGIEVVGSLVGTRQDLTEAFQFAAEGKVVPKVALRPLADINTIFTEMEEGKIRGRMVIDFRH

SEQ ID NO: 39 - DNA, 987 bp, Escherichia coli
  misc_feature (1)..(987): Coding Sequence for E. coli yiaE 2-keto acid dehydrogenase, from GenBank AE005174
atggagagaagcatgaagccgtccgttatcctctacaaagccttacctgatgatttactgcaacgcctgcaagagcatttcaccgttcaccaggtggcaaacctcagcccacaaaccgtcgaacaaaatgcagcaatttttgccgaagctgaaggtttactgggttcaaacgagaatgttgatgccgcattgctggaaaaaatgccgaaactgcgtgccacatcaacgatctccgtcggctatgacaattttgatgtcgatgcgcttaccgcccgaaaaattctgctgatgcacacgccaaccgtcttaacagaaaccgtcgccgatacgctgatggcgctggtgttgtctaccgctcgtcgggttgtggaggtagcagaacgggtaaaagcaggcgaatggaccgcgagcataggcccggactggtacggcactgacgttcaccataaaacactgggcattgtcgggatgggacggatcggtatggcgctggcacaacgtgcgcactttggcttcaacatgcccatcctctataacgcgcgccgccaccataaagaagcagaagaacgcttcaacgcccgctactgcgatttggatacactgttacaagagtcagatttcgtttgcctgatcctgccgttaactgatgagacgcatcatctgtttggcgcagaacaattcgccaaaatgaaatcctccgccattttcattaatgccggacgtggcccggtggttgacgaaaatgcactgatcgcagcattgcagaaaggggaaattcacgccgccgggctggatgtcttcgaacaagagccactttccgtagattcgccgttgctctcaatggccaacgtcgtcgcagtaccgcatattggatctgccacccatgagacgcgttatggcatggccgcctgtgccgtggataatttgattgatgcgttacaaggaaaggttgagaagaactgtgtgaatccgcacgtcgcggactaa

SEQ ID NO: 40 - protein, 328 aa, Escherichia coli
  MISC_FEATURE (1)..(328): YiaE 2-keto acid dehydrogenase (Genbank Accession No. AAG58702)
MERSMKPSVILYKALPDDLLQRLQEHFTVHQVANLSPQTVEQNAAIFAEAEGLLGSNENVDAALLEKMPKLRATSTISVGYDNFDVDALTARKILLMHTPTVLTETVADTLMALVLSTARRVVEVAERVKAGEWTASIGPDWYGTDVHHKTLGIVGMGRIGMALAQRAHFGFNMPILYNARRHHKEAEERFNARYCDLDTLLQESDFVCLILPLTDETHHLFGAEQFAKMKSSAIFINAGRGPVVDENALIAALQKGEIHAAGLDVFEQEPLSVDSPLLSMANVVAVPHIGSATHETRYGMAACAVDNLIDALQGKVEKNCVNPHVAD

SEQ ID NO: 41 - DNA, 939 bp, Escherichia coli
  misc_feature (1)..(939): Coding Sequence for E. coli ycdW 2-Keto acid Dehydrogenase, from GenBank AP009048
atggatatcatcttttatcacccaacgttcgatacccaatggtggattgaggcactgcgcaaagctattcctcaggcaagagtcagagcatggaaaagcggagataatgactctgctgattatgctttagtctggcatcctcctgttgaaatgctggcagggcgcgatcttaaagcggtgttcgcactcggggccggtgttgattctattttgagcaagctacaggcacaccctgaaatgctgaacccttctgttccactttttcgcctggaagataccggtatgggcgagcaaatgcaggaatatgctgtcagtcaggtgctgcattggtttcgacgttttgacgattatcgcatccagcaaaatagttcgcattggcaaccgctgcctgaatatcatcgggaagattttaccatcggcattttgggcgcaggcgtactgggcagtaaagttgctcagagtctgcaaacctggcgctttccgctgcgttgctggagtcgaacccgtaaatcgtggcctggcgtgcaaagctttgccggacgggaagaactgtctgcatttctgagccaatgtcgggtattgattaatttgttaccgaatacccctgaaaccgtcggcattattaatcaacaattactcgaaaaattaccggatggcgcgtatctcctcaacctggcgcgtggtgttcatgttgtggaagatgacctgctcgcggcgctggatagcggcaaagttaaaggcgcaatgttggatgtttttaatcgtgaacccttaccgcctgaaagtccgctctggcaacatccacgcgtgacgataacaccacatgtcgccgcgattacccgtcccgctgaagctgtggagtacatttctcgcaccattgcccagctcgaaaaaggggagagggtctgcgggcaagtcgaccgcgcacgcggctactaa

SEQ ID NO: 42 - protein, 312 aa, Escherichia coli
  MISC_FEATURE (1)..(312): YcdW 2-Keto acid Dehydrogenase (Genbank Accession No. BAA35814)
MDIIFYHPTFDTQWWIEALRKAIPQARVRAWKSGDNDSADYALVWHPPVEMLAGRDLKAVFALGAGVDSILSKLQAHPEMLNPSVPLFRLEDTGMGEQMQEYAVSQVLHWFRRFDDYRIQQNSSHWQPLPEYHREDFTIGILGAGVLGSKVAQSLQTWRFPLRCWSRTRKSWPGVQSFAGREELSAFLSQCRVLINLLPNTPETVGIINQQLLEKLPDGAYLLNLARGVHVVEDDLLAALDSGKVKGAMLDVFNREPLPPESPLWQHPRVTITPHVAAITRPAEAVEYISRTIAQLEKGERVCGQVDRARGY

SEQ ID NO: 43 - DNA, 1587 bp, Pseudomonas putida
  misc_feature (1)..(1587): Coding Sequence for P. putida mdlC 2-keto acid decarboxylase, from GenBank AY143338
tcacttcaccgggcttacggtgcttacttcgataagtaccgggcctttggcagaaagcgcttcttgtagcgaacccttgagctgctcaaggttgtcggctttcagcgcttggacaccatagcccttggcgagtgcgcggaagtcgatccctggcacatccagcccaggaacgttttctgcttcgagaacgccggcaaaccatcgcaacgcaccgtaggtgccgttgttcatgatcacgaagatagtggggatgttgtactgagctgcagtccacaacgcactaatgctgtagttcgccgatccgtcgccaatgacggcgatgacttgtcgctcgggttctgcgagttgaacgccaattgctgcaggcagggcgaagcccagtccgccagctgcacagaagtagtagctaccagggttgcgcatgttcaggcgctgccacatttgggcggtcgttgaagtcgactcgttcaggtaaatcgcattctccggggccatgtcgttcagtgtgtcgaacactgtctctgggtgaagtcggccagcgtcttggtcaaccttcgcgggttccggagctgcagttgggagctggcggctgctctcttcaaccaagttggcaagagcgctagccatcgcaccaatgtctgccacgatcgcatcgcccattggcgcgcgtgcagcttcgagcgggtcgcaggtcaccgaaatcaatcgcgtgccaggtttgagatattgacctgggtcgtattggtggtaacggaacactggagcgccgattaccaaaaccacatcgtgaccttcgagcagctgagaaatcgctgcgatgccagctggcatcaatccacggaagcaaggatgacgggtagggaatgggcagcgtggagcggatggcgcaacccaaaccggagctttgaggcgttcggccaacatgacgcagtctgcgttcgcatttgctgcgtcgacgtccgggcccaggacgatcgccgggttggatgcgctgttgagagctttcaccagaatatcgagatcctggtcgttcaggcgtactgatgaactgacatggcgatcaaaaaggtggtgggactgaggatcagcatccttatcccaatcgtcatatggcaccgaaagatagacagggccttgtggcgccatgcttgccatatggatagccctgctcatcgcatgagggacttctgctgcgcttgcgggctcgtagctccatttgacaagtggtcgtggcaggttggcggcatcgacgttggtcagcagagcttcaacgccaatcatcgccctggtctgctggccggcagtgacgatcagcggggaatgtgagttccaggcgttactgagtgcacccatagcattgccggtaccagcagcagaatgcaggttaatgaaagccggcttccgactggcttgcgcatagccgtctgcaatgcccaccacacacgcttcctgcaaagccaggatgtatcgaaagtcctctggaaagtccttcaaaaacgggagctcgttcgagccaggattgccgaagaccgtatcgatgccttgacgtcgcaagagttcgtatgtggtgccgtgtaccgaagccat

SEQ ID NO: 44 - protein, 528 aa, Pseudomonas putida [sequence omitted]

(19) United States
(12) Patent Application Publication
(10) Pub. No.: US 2011/0076730 A1
(43) Pub. Date: Mar. 31, 2011

(54) MICROBIAL SYNTHESIS OF D-1,2,4-BUTANETRIOL

(75) Inventors: John W. Frost, Okemos, MI (US); Wei Niu, Spring Park, MN (US)

(73) Assignee: Board of Trustees of Michigan State University, East Lansing, MI (US)

(21) Appl. No.: 12/374,367

(22) PCT Filed: Jul. 19, 2007
(86) PCT No.: PCT/US07/16384

§ 371 Date: Dec. 31, 2009

Related U.S. Application Data
(60) Provisional application No. 60/831,964, filed on Jul. 19, 2006.

(51) Int. Cl. C12P 13/04; C12P 7/18; C07C 201/02; C12N 9/04; C07H 21/04; C12N 15/63; C12N 9/88; C12N 5/10; C12N 1/00; C12P 7/58; C12P 7/52; C12N 9/02; C12N 9/10; C07K 16/40; C07K 16/44; G01N 33/53

(52) U.S. Cl. 435/106; 435/158; 558/484; 435/190; 536/23.2; 435/320.1; 435/232; 435/419; 435/243; 435/137; 435/141; 435/189; 435/193; 530/387.9; 530/387.5; 436/501

(57) ABSTRACT

Improved enzyme systems, recombinant cells, and processes employing the same to produce biosynthetic D-1,2,4-butanetriol; D-1,2,4-butanetriol prepared thereby and derivatives thereof; D-1,2,4-butanetriol trinitrate prepared therefrom; and enzymes and genes useful in the enzyme systems and recombinant cells.

### CROSS-REFERENCE TO RELATED APPLICATIONS

This application claims the benefit of U.S. Provisional Application 60/831,964, filed on Jul. 19, 2006. The disclosure of the above application is incorporated herein by reference.

### SPONSORSHIP

This invention was made with Government support under Contract N00014-00-1-0825, awarded by the Office of Naval Research, and with support from the National Science Foundation. The Government may have certain rights in this invention.

### FIELD

The present disclosure relates to methods and materials for biosynthesis of 1,2,4-butanetriol and for production of 1,2,4-butanetriol trinitrate therefrom, as well as methods and materials for biosynthesis of compounds identified as by-products of 1,2,4-butanetriol biosynthetic systems hereof.

### BACKGROUND

The statements in this section merely provide background information related to the present disclosure and may not constitute prior art.

1,2,4-butanetriol is a chiral polyhydroxyl alcohol useful in forming energetic compounds, as well as bioactive agents, e.g., beta-acaridial pheromone. Racemic D,L-1,2,4-butanetriol can be nitrated to form the energetic material D,L-1,2,4-butanetriol trinitrate, which is less shock sensitive, more thermally stable and less volatile than the conventional energetic plasticizer, nitroglycerin. (CPIA/M3Solid Propellant Ingredients Manual; The Johns Hopkins University, Chemical Propulsion Information Agency: Whiting School of Engineering, Columbia, Md., 2000.) Although individual enantiomers of 1,2,4-butanetriol can be nitrated, the racemic mixture of D,L-1,2,4-butanetriol is typically employed as the synthetic precursor of 1,2,4-butanetriol trinitrate. 1,2,4-butantriol trinitrate is an energetic plasticizer with both civilian and military application potentials. V. Lindner, Explosives. In Kirk-Othmer Encyclopedia of Chemical Technology Online. (Wiley, New York, 1994). Thus, substitution of nitroglycerin with 1,2,4-butanetriol trinitrate as an energetic material promises to not only reduce hazards associated with such manufacturing and operating processes, but also to improve the operating range of the final product.

However, the limited availability of 1,2,4-butanetriol has limited the large-scale production of 1,2,4-butanetriol trinitrate. 1,2,4-Butanetriol is currently commercially manufactured by high pressure catalytic hydrogenation of D,L-malic acid, using NaBH₄ reduction of esterified D,L-malic acid, e.g., dimethyl malate, in a mixture of C₂₋₆ alcohols and tetrahydrofuran (FIG. 1a). (U.S. Pat. No. 6,479,714, Schofield et al., issued Nov. 12, 2002; International Publication WO 99/44976, Ikai, et al., published Sep. 10, 1999.) This chemosynthetic route also produces a variety of byproducts and for each ton of D,L-1,2,4-butanetriol synthesized, multiple tons of byproducts are generated, since this reaction generates 2-5 kg of borate salts for every kg of dimethyl malate being reduced. See, e.g., International Publication WO 98/08793, Monteith et al., issued Mar. 5, 1998; International Publication WO 99/44976, lkai et al., issued Sep. 10, 1999; H. Adkins & H. R. Billica, J. Am. Chem. Soc. 70:3121 (1948); U.S. Pat. No. 4,973,769, Mueller et al, issued Nov. 27, 1990; and U.S. Pat. No. 6,355,848, Antons et al., issued Mar. 12, 2002. The cost of proper disposal of the byproduct salt stream combined with the expense of employing stoichiometric amounts of NaBH₄ limit the application of this reaction to the production of small volumes of 1,2,4-butanetriol.

As a result, more economical and environmentally safer, biosynthetic techniques for obtaining D-, L-, and D,L-1,2,4-butanetriol have recently been developed, wherein the D-isomer is obtained by bioconversion of D-xylose or D-xylonic acid, and the L-isomer obtained by bioconversion of L-arabinose or L-arabinonic acid (FIG. 1b); and biosynthesis of each enantiomer has been successfully exemplified using a two-microbe process via the intermediacy of D-xylonic acid or L-arabinonic acid. See, e.g., W. Niu et al., Microbial synthesis of the energetic material precursor 1,2,4-butanetriol. J. Am. Chem. Soc. 125:12998-12999 (2003). Nevertheless, large-scale application of these biosynthetic routes is economically challenging due to the large amount of nutrient supplements that have been found important for optimizing strain cultivations and to the desirability of biosynthetic intermediates purification to maximize 1,2,4-butanetriol production. Thus, it would be advantageous to provide a single recombinant cell, capable of biosynthesis of 1,2,4-butanetriol, by growth on inexpensive media, e.g., a carbon-source-supplemented, minimal salts medium.

Although both the xylose/xylonate and arabinose/arabinonate routes can be used to obtain 1,2,4-butantriol, D-xylose, and D-xylonic acid, are economically advantageous relative to, e.g., L-arabinose, or L-arabinonic acid, in part due to the fact that D-xylose is more prevalent in low-cost, carbon source starting materials such as the hemicelluloses found in wood and plant fiber waste. For example, this is reflected in price comparison of commercially available pentoses, which shows that L-arabinose costs about twice as much as D-xylose (e.g., see Sigma-Aldrich product no. X1500 for 10 mg of >99% pure D-xylose at US$6.25, and product no. A3256 for 10 mg of >99% pure L-arabinose at US$13.00). As a result, it would be desirable to obtain 1,2,4-butanetriol biosynthesis systems that utilize a D-xylose, or D-xylonic acid, source, and that are useful for producing commercial yields of 1,2,4-butanetriol.

However, recently it has also been unexpectedly discovered that various desirable host cells for commercial scale 1,2,4-butanetriol biosynthesis contain native biocatalytic activities that are responsible for decreasing the actual yield of 1,2,4-butanetriol, obtainable from D-xylose or D-xylonic acid, to a level that is substantially below the theoretical maximum yield. As a result, it would be advantageous to provide improved host cells for 1,2,4-butanetriol biosynthesis that utilize D-xylose, or D-xylonic acid, but in which the yield can be increased by inhibiting or inactivating such carbon-diverting biocatalytic activities.

Major challenges to such further improvement of D-1,2,4-butanetriol biosynthesis systems lie in the lack of genetic information on the D-xylose dehydrogenase enzyme catalyzing the first step in the artificial biosynthetic pathway (FIG. 1b) and the existence of the above-described, unelucidated catabolic background in the microbial host cell.

Thus, it would be further advantageous to provide specific D-xylose dehydrogenase genes encoding enzymes having an efficient ability to convert D-xylose to D-xylonic acid, and that can be expressed in host cells useful for D-1,2,4-butanetriol biosynthesis, as well as to characterize the mechanism of the undesirable catabolic reactions in such as way as to provide a technique for controlling it.

### SUMMARY

In various embodiments, the present invention provides improved host cells that are capable of bioconverting a D-xylose, or D-xylonic acid, source to 1,2,4-butanetriol, and in which one or more carbon-diverting biocatalytic activity is inhibited or inactivated. In some embodiments, the carbon-diverting biocatalytic activity that is inhibited or inactivated is a 3-deoxy-D-glycero-pentulosonic acid aldolase that is capable of splitting 3-deoxy-D-glycero-pentulosonic acid to form pyruvate and glycolaldehyde. The present invention also provides specific, novel D-xylose dehydrogenases and their coding sequences. The present invention further provides:

Processes for preparing D-1,2,4-butanetriol, comprising (A) providing (1) a recombinant cellular entity containing a 1,2,4-butanetriol biosynthetic enzyme system that comprises (a) a D-xylose dehydrogenase, (b) a D-xylonic acid dehydratase, (c) a 2-keto acid decarboxylase, and (d) an alcohol dehydrogenase, wherein the cellular entity is one that has been manipulated to inhibit or inactivate a 3-deoxy-D-glycero-pentulosonic acid aldolase polypeptide or nucleic acid thereof; and (2) a xylose source that is capable, under conditions in which the enzyme system can operate to produce 1,2,4-butanetriol, of providing D-xylose to the D-xylose dehydrogenase enzyme; and (B) placing the cellular entity and the xylose source under conditions in which the enzyme system can produce 1,2,4-butanetriol from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate,
(3) the 2-keto acid decarboxylase to convert resulting 3-deoxy-D-glycero-pentulosonate to 3,4-dihydroxy-D-butanal, and
(4) the alcohol dehydrogenase to convert resulting 3,4-dihydroxy-D-butanal to D-1,2,4-butanetriol;
   thereby preparing D-1,2,4-butanetriol.

Processes for preparing D-1,2,4-butanetriol, comprising (A) providing (1) a recombinant cellular entity containing a 1,2,4-butanetriol biosynthetic enzyme system that comprises (a) a D-xylose dehydrogenase comprising the amino acid sequence of any one of SEQ ID NO:2, SEQ ID NO:4, or a conservative-substituted variant of or homologous polypeptide to SEQ ID NO:2 or SEQ ID NO:4 and having D-xylose dehydrogenase activity, (b) a D-xylonic acid dehydratase, (c) a 2-keto acid decarboxylase, and (d) an alcohol dehydrogenase, and (2) a xylose source that is capable, under conditions in which the enzyme system can operate to produce 1,2,4-butanetriol, of providing D-xylose to the D-xylose dehydrogenase enzyme; and (B) placing the cellular entity and the xylose source under conditions in which the enzyme system can produce 1,2,4-butanetriol from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate,
(3) the 2-keto acid decarboxylase to convert resulting 3-deoxy-D-glycero-pentulosonate to 3,4-dihydroxy-D-butanal, and
(4) the alcohol dehydrogenase to convert resulting 3,4-dihydroxy-D-butanal to D-1,2,4-butanetriol;
   thereby preparing D-1,2,4-butanetriol.

Process for preparing D-1,2,4-butanetriol, comprising (A) providing (1) a recombinant cellular entity containing a 1,2,4-butanetriol biosynthetic enzyme system that comprises (a) a D-xylose dehydrogenase, (b) a D-xylonic acid dehydratase comprising (i) the amino acid sequence of any one of SEQ ID NO:6, SEQ ID NO:8, or a conservative-substituted variant of or homologous polypeptide to SEQ ID NO:6 or SEQ ID NO:8 and having D-xylonate dehydratase activity, or (ii) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or a conservative-substituted variant thereof or homologous polypeptide thereto, (c) a 2-keto acid decarboxylase, and (d) an alcohol dehydrogenase, and (2) a xylose source that is capable, under conditions in which the enzyme system can operate to produce 1,2,4-butanetriol, of providing D-xylose to the D-xylose dehydrogenase enzyme; and (B) placing the cellular entity and the xylose source under conditions in which the enzyme system can produce 1,2,4-butanetriol from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate,
(3) the 2-keto acid decarboxylase to convert resulting 3-deoxy-D-glycero-pentulosonate to 3,4-dihydroxy-D-butanal, and
(4) the alcohol dehydrogenase to convert resulting 3,4-dihydroxy-D-butanal to D-1,2,4-butanetriol;
   thereby preparing D-1,2,4-butanetriol.

Processes for preparing D-1,2,4-butanetriol, comprising (A) providing (1) a recombinant cellular entity containing a 1,2,4-butanetriol biosynthetic enzyme system that comprises (a) a D-xylonic acid dehydratase comprising (i) the amino acid sequence of any one of SEQ ID NO:6, SEQ ID NO:8, or a conservative-substituted variant of or homologous polypeptide to SEQ ID NO:6 or SEQ ID NO:8 and having D-xylonate dehydratase activity, or (ii) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or a conservative-substituted variant thereof or homologous polypeptide thereto, (b) a 2-keto acid decarboxylase, and (c) an alcohol dehydrogenase, and (2) a xylonate source that is capable, under conditions in which the enzyme system can operate to produce 1,2,4-butanetriol, of providing D-xylonate to the D-xylonic acid dehydratase enzyme; and (B) placing the cellular entity and the xylonate source under conditions in which the enzyme system can produce 1,2,4-butanetriol from D-xylonate, and in which the xylonate source provides D-xylonate to the D-xylonic acid dehydratase enzyme, the enzyme system operating under the conditions by action of
(1) the D-xylonic acid dehydratase to convert resulting D-xylonate to 3-deoxy-D-glycero-pentulosonate,
(2) the 2-keto acid decarboxylase to convert resulting 3-deoxy-D-glycero-pentulosonate to 3,4-dihydroxy-D-butanal, and
(3) the alcohol dehydrogenase to convert resulting 3,4-dihydroxy-D-butanal to D-1,2,4-butanetriol;
   thereby preparing D-1,2,4-butanetriol.

Processes for preparing D-1,2,4-butanetriol, comprising (A) providing (1) a recombinant cellular entity containing a 1,2,4-butanetriol biosynthetic enzyme system that comprises (a) a D-xylonic acid dehydratase, (b) a 2-keto acid decarboxylase, and (c) an alcohol dehydrogenase, wherein the cellular entity is one that has been manipulated to inhibit or inactivate a 3-deoxy-D-glycero-pentulosonic acid aldolase polypeptide or nucleic acid thereof; and (2) a xylonate source that is capable, under conditions in which the enzyme system can operate to produce 1,2,4-butanetriol, of providing D-xylonate to the D-xylonic acid dehydratase enzyme; and (B) placing the cellular entity and the xylose source under conditions in which the enzyme system can produce 1,2,4-butanetriol from D-xylonic acid, and in which the xylonate source provides D-xylonate to the D-xylonate dehydratase enzyme, the enzyme system operating under the conditions by action of
(1) the D-xylonic acid dehydratase to convert D-xylonate to 3-deoxy-D-glycero-pentulosonate,
(2) the 2-keto acid decarboxylase to convert resulting 3-deoxy-D-glycero-pentulosonate to 3,4-dihydroxy-D-butanal, and
(3) the alcohol dehydrogenase to convert resulting 3,4-dihydroxy-D-butanal to D-1,2,4-butanetriol;
   thereby preparing D-1,2,4-butanetriol.

Such processes in which the recombinant cellular entity comprises a single cell that contains the enzyme system; such processes in which the cell is a microbial or plant cell; such processes that further include recovering D-1,2,4-butanetriol prepared thereby; D-1,2,4-Butanetriol prepared by such processes; processes for preparing 1,2,4-butanetriol trinitrate therefrom; D-1,2,4-Butanetriol trinitrate prepared by such a process;

D-xylose dehydrogenase enzymes comprising the amino acid sequence of any one of SEQ ID NO:2, SEQ ID NO:4, or a conservative-substituted variant of or homologous polypeptide to SEQ ID NO:2 or SEQ ID NO:4 and having D-xylose dehydrogenase activity; nucleic acids encoding such enzymes, and nucleic acids comprises the base sequence of any one of SEQ ID NO:1, SEQ ID NO:3, or a homologous polynucleotide to SEQ ID NO:1 or SEQ ID NO:3;

D-xylonic acid dehydratase enzymes comprising the amino acid sequence of any one of: SEQ ID NO:6; SEQ ID NO:8; a conservative-substituted variant of or homologous polypeptide to SEQ ID NO:6 or SEQ ID NO:8; a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end; or a conservative-substituted variant of or homologous polypeptide to the P. fragi D-xylonate dehydratase amino acid sequence; nucleic acids encoding such enzymes, and nucleic acids comprises the base sequence of any one of SEQ ID NO:1, SEQ ID NO:3, or a homologous polynucleotide to SEQ ID NO:1 or SEQ ID NO:3.

Use of such an enzyme in a D-1,2,4-butanetriol biosynthetic enzyme system;

Isolated or recombinant 1,2,4-butanetriol biosynthetic enzyme systems that comprise (A) a D-xylose dehydrogenase comprising the amino acid sequence of any one of SEQ ID NO:2, SEQ ID NO:4, or a conservative-substituted variant of or homologous polypeptide to SEQ ID NO:2 or SEQ ID NO:4 and having D-xylose dehydrogenase activity, (B) a D-xylonic acid dehydratase, (C) a 2-keto acid decarboxylase, and (D) an alcohol dehydrogenase, the enzyme system being capable of catalyzing the conversion of D-xylose to D-1,2,4-butanetriol;

Isolated or recombinant 1,2,4-butanetriol biosynthetic enzyme systems that comprise (A) a D-xylose dehydrogenase, (B) a D-xylonic acid dehydratase comprising (1) the amino acid sequence of any one of SEQ ID NO:6, SEQ ID NO:8, or a conservative-substituted variant of or homologous polypeptide to SEQ ID NO:6 or SEQ ID NO:8 and having D-xylonate dehydratase activity, or (2) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or a conservative-substituted variant thereof or homologous polypeptide thereto, (C) a 2-keto acid decarboxylase, and (D) an alcohol dehydrogenase, the enzyme system being capable of catalyzing the conversion of D-xylose to D-1,2,4-butanetriol.

Isolated or recombinant 2,4-butanetriol biosynthetic enzyme systems that comprise (A) a D-xylonic acid dehydratase comprising (1) the amino acid sequence of any one of SEQ ID NO:6, SEQ ID NO:8, or a conservative-substituted variant of or homologous polypeptide to SEQ ID NO:6 or SEQ ID NO:8 and having D-xylonate dehydratase activity, or (2) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or a conservative-substituted variant thereof or homologous polypeptide thereto, (B) a 2-keto acid decarboxylase, and (C) an alcohol dehydrogenase, the enzyme system being capable of catalyzing the conversion of D-xylonate to D-1,2,4-butanetriol.

Recombinant cellular entities that comprises such an enzyme system; such entitites that comprise a single cell that contains the enzyme system; such cells that are recombinant 3-deoxy-D-glycero-pentulosonic acid aldolase “minus” DgPu⁻ cells;

3-Deoxy-D-glycero-pentulosonate aldolase knock-out vectors comprising a polynucleotide containing a base sequence from any one of SEQ ID NO:11, SEQ ID NO:13, or nt55-319 of SEQ ID NO:11, wherein the vector is capable of inserting into or recombining with a genomic copy of a 3-deoxy-D-glycero-pentulosonate aldolase gene in such a manner as to inactivate the gene or its encoded aldolase.

Recombinant DgPu⁻ (3-deoxy-D-glycero-pentulosonate aldolase “minus”) cells;

Processes for preparing 3-deoxy-D-glycero-pentanoic acid, comprising (A) providing (1) a recombinant cellular entity containing a 3-deoxy-D-glycero-pentanoic acid biosynthetic enzyme system that comprises (a) a D-xylose dehydrogenase, (b) a D-xylonic acid dehydratase, and (c) a 2-keto-acid reductase, and (2) a xylose source that is capable, under conditions in which the enzyme system can operate to produce 3-deoxy-D-glycero-pentanoic acid, of providing D-xylose to the D-xylose dehydrogenase enzyme; and (B) placing the cellular entity and the xylose source under conditions in which the enzyme system can produce 3-deoxy-D-glycero-pentanoic acid from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate, and
(3) the 2-keto acid dehydrogenase (reductase) to convert resulting 3-deoxy-D-glycero-pentulosonate to 3-deoxy-D-glycero-pentanoic acid,
   thereby preparing 3-deoxy-D-glycero-pentanoic acid.

Processes for preparing 3-deoxy-D-glycero-pentanoic acid, comprising (A) providing (1) a recombinant cellular entity containing a 3-deoxy-D-glycero-pentanoic acid biosynthetic enzyme system that comprises (a) a D-xylonic acid dehydratase, and (b) a 2-keto-acid reductase, and (2) a xylonate source that is capable, under conditions in which the enzyme system can operate to produce 3-deoxy-D-glycero-pentanoic acid, of providing D-xylonate to the D-xylonate dehydratase enzyme; and (B) placing the cellular entity and the xylose source under conditions in which the enzyme system can produce 3-deoxy-D-glycero-pentanoic acid from D-xylonate, and in which the xylonate source provides D-xylonate to the D-xylonate dehydratase enzyme, the enzyme system operating under the conditions by action of
(1) the D-xylonic acid dehydratase to convert D-xylonate to 3-deoxy-D-glycero-pentulosonate, and
(2) the 2-keto acid dehydrogenase (reductase) to convert resulting 3-deoxy-D-glycero-pentulosonate to 3-deoxy-D-glycero-pentanoic acid,
   thereby preparing 3-deoxy-D-glycero-pentanoic acid.

Processes for preparing D-3,4-dihydroxy-butanoic acid, comprising:

(A) providing (1) a recombinant cellular entity containing a D-3,4-dihydroxy-butanoic acid biosynthetic enzyme system that comprises (a) a D-xylose dehydrogenase, (b) a D-xylonic acid dehydratase, and (c) a 2-keto-acid decarboxylase, and (d) an aldehyde dehydrogenase, and (2) a xylose source that is capable, under conditions in which the enzyme system can operate to produce D-3,4-dihydroxy-butanoic acid, of providing D-xylose to the D-xylose dehydrogenase enzyme; and (B) placing the cellular entity and the xylose source under conditions in which the enzyme system can produce D-3,4-dihydroxy-butanoic acid from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate, and
(3) the 2-keto acid decarboxylase to convert resulting 3-deoxy-D-glycero-pentulosonate to 3,4-dihydroxy-D-butanal, and
(4) the aldehyde dehydrogenase to convert resulting 3,4-dihydroxy-D-butanal to D-3,4-dihydroxy-butanoic acid,
   thereby preparing D-3,4-dihydroxy-butanoic acid.

Processes for preparing D-3,4-dihydroxy-butanoic acid, comprising

(A) providing (1) a recombinant cellular entity containing a D-3,4-dihydroxy-butanoic acid biosynthetic enzyme system that comprises (a) a D-xylonic acid dehydratase, and (b) 2-keto-acid decarboxylase, and (c) an aldehyde dehydrogenase, and (2) a xylonate source that is capable, under conditions in which the enzyme system can operate to produce D-3,4-dihydroxy-butanoic acid, of providing D-xylonate to the D-xylonate dehydratase enzyme; and (B) placing the cellular entity and the xylonate source under conditions in which the enzyme system can produce D-3,4-dihydroxy-butanoic acid from D-xylonic acid, and in which the xylonate source provides D-xylonate to the D-xylonate dehydratase enzyme, the enzyme system operating under the conditions by action of
(1) the D-xylonic acid dehydratase to convert D-xylonate to 3-deoxy-D-glycero-pentulosonate, and
(2) the 2-keto acid decarboxylase to convert resulting 3-deoxy-D-glycero-pentulosonate to 3,4-dihydroxy-D-butanal, and
(3) the aldehyde dehydrogenase to convert resulting 3,4-dihydroxy-D-butanal to D-3,4-dihydroxy-butanoic acid,
   thereby preparing D-3,4-dihydroxy-butanoic acid.

Processes for preparing (4S)-2-amino-4,5-dihydroxy pentanoic acid, comprising (A) providing (1) a recombinant cellular entity containing a 3-deoxy-D-glycero-pentanoic acid biosynthetic enzyme system that comprises (a) a D-xylose dehydrogenase, (b) a D-xylonic acid dehydratase, and (c) a 2-keto acid transaminase, and (2) a xylose source that is capable, under conditions in which the enzyme system can operate to produce (4S)-2-amino-4,5-dihydroxy pentanoic acid, of providing D-xylose to the D-xylose dehydrogenase enzyme; and (B) placing the cellular entity and the xylose source under conditions in which the enzyme system can produce (4S)-2-amino-4,5-dihydroxy pentanoic acid from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate, and
(3) the 2-keto acid transaminase to convert resulting 3-deoxy-D-glycero-pentulosonate to (4S)-2-amino-4,5-dihydroxy pentanoic acid,
   thereby preparing (4S)-2-amino-4,5-dihydroxy pentanoic acid.

Processes for preparing (4S)-2-amino-4,5-dihydroxy pentanoic acid, comprising (A) providing (1) a recombinant cellular entity containing a 3-deoxy-D-glycero-pentanoic acid biosynthetic enzyme system that comprises (a) a D-xylonic acid dehydratase, and (b) a 2-keto acid transaminase, and (2) a xylonate source that is capable, under conditions in which the enzyme system can operate to produce (4S)-2-amino-4,5-dihydroxy pentanoic acid, of providing D-xylonate to the D-xylonate dehydratase enzyme; and (B) placing the cellular entity and the xylose source under conditions in which the enzyme system can produce (4S)-2-amino-4,5-dihydroxy pentanoic acid from D-xylonic acid, and in which the xylonate source provides D-xylonate to the D-xylonate dehydratase enzyme, the enzyme system operating under the conditions by action of
(1) the D-xylonic acid dehydratase to convert D-xylonate to 3-deoxy-D-glycero-pentulosonate, and
(2) the 2-keto acid transaminase to convert resulting 3-deoxy-D-glycero-pentulosonate to (4S)-2-amino-4,5-dihydroxy pentanoic acid,
   thereby preparing (4S)-2-amino-4,5-dihydroxy pentanoic acid.

Such processes in which the cellular entity comprises a single cell that contains the enzyme system; such processes in which the cell is a recombinant DgPu⁻ cell;

3-Deoxy-D-glycero-pentanoic acid, D-3,4-dihydroxy-butanoic acid, and/or (4S)-2-amino-4,5-dihydroxy pentanoic acid prepared such a process

Isolated or recombinant 3-deoxy-D-glycero-pentanoic acid biosynthetic enzyme systems that comprise (A) a D-xylose dehydrogenase, (B) a D-xylonic acid dehydratase, and (C) a 2-keto-acid reductase, the enzyme system being capable of catalyzing the conversion of D-xylose to 3-deoxy-D-glycero-pentanoic acid;

Isolated or recombinant 3-deoxy-D-glycero-pentanoic acid biosynthetic enzyme systems that comprise (A) a D-xylonic acid dehydratase, and (B) a 2-keto-acid reductase, the enzyme system being capable of catalyzing the conversion of D-xylonate to 3-deoxy-D-glycero-pentanoic acid;

Isolated or recombinant D-3,4-dihydroxy-butanoic acid biosynthetic enzyme system that comprise: (A) a D-xylose dehydrogenase, (B) a D-xylonic acid dehydratase, and (C) a 2-keto-acid decarboxylase, and (D) an aldehyde dehydrogenase, the enzyme system being capable of catalyzing the conversion of D-xylose to D-3,4-dihydroxy-butanoic acid;

Isolated or recombinant D-3,4-dihydroxy-butanoic acid biosynthetic enzyme systems that comprise (A) a D-xylonic acid dehydratase, (B) a 2-keto-acid decarboxylase, and (C) an aldehyde dehydrogenase, the enzyme system being capable of catalyzing the conversion of D-xylonate to D-3,4-dihydroxy-butanoic acid.

Isolated or recombinant (4S)-2-amino-4,5-dihydroxy pentanoic acid biosynthetic enzyme systems that comprise (A) a D-xylose dehydrogenase, (B) a D-xylonic acid dehydratase, and (C) a 2-keto acid transaminase, the enzyme system being capable of catalyzing the conversion of D-xylose to (4S)-2-amino-4,5-dihydroxy pentanoic acid.

Isolated or recombinant (4S)-2-amino-4,5-dihydroxy pentanoic acid biosynthetic enzyme systems that comprise (A) a D-xylonic acid dehydratase, and (B) a 2-keto acid transaminase, the enzyme system being capable of catalyzing the conversion of D-xylonate to (4S)-2-amino-4,5-dihydroxy pentanoic acid.

Recombinant cellular entity that comprise such an enzyme system; and those in which the cellular entity comprises a single cell that contains the enzyme system; and those in which the cell is a recombinant DgPu⁻ cell;

Processes for screening for candidate enzyme-encoding polynucleotides, comprising (A) providing (1) a nucleic acid or nucleic acid analog probe comprising a nucleobase sequence identical to that of about 20 or more contiguous nucleotides of a coding sequence that encodes an enzyme polypeptide having any one of (a) the amino acid sequence of any one of SEQ ID NOs:2, 4, 6, 8, 10, 12, or 14, or (b) the amino acid sequence of residues 19-319 of SEQ ID NO:12, or (c) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or (d) the amino acid sequence of a biocatalytic activity retaining conservative substituted variant of or homologous amino acid sequence to any of (a), (b), or (c); and (2) a test sample comprising or suspected of comprising at least one target nucleic polynucleotide to which such a probe can specifically bind; (B) contacting the probe with the test sample under conditions in which the probe can specifically hybridize to a target polynucleotide, if present, to form a probe-target polynucleotide complex, and (C) detecting whether or not any probe-target polynucleotide complexes were formed thereby, wherein a target polynucleotide that was identified as part of a complex is thereby identified as a candidate enzyme-encoding polynucleotide.

Antibodies having specificity for an epitope of (A) an enzyme polypeptide having any one of (1) the amino acid sequence of any one of SEQ ID NOs:2, 4, 6, 8, 10, 12, or 14, or (2) the amino acid sequence of residues 19-319 of SEQ ID NO:12, or (3) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or (4) the amino acid sequence of a biocatalytic activity-retaining conservative substituted variant of or homologous amino acid sequence to any of (1), (2), or (3); or (B) a polynucleotide or nucleic acid analog having a base sequence encoding such an enzyme polypeptide (A).

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 illustrates synthetic routes to 1,2,4-butanetriol. (1a) Current commercial synthesis of 1,2,4-butanetriol from dimethyl malate using sodium borohydride and tetrahydrofuran in a C₂₋₆ alcohol(s). (1b and 1c) Biosynthetic pathway of D- and L-1,2,4-butanetriol. Enzymes: a) D-xylose dehydrogenase; a′) L-arabinose dehydrogenase; b) D-xylonic acid dehydratase; b′) L-arabinonic acid dehydratase; c) 2-keto acid decarboxylase; d) alcohol dehydrogenase.

FIG. 2 illustrates steps involved in the isolation of the partial coding sequence of the Pseudomonas fragi (ATCC 4973) D-xylonic acid dehydratase. (2a) SDS-PAGE of the D-xylonic acid dehydratase purified from P. fragi. (2b) N-terminal sequences of trypsin-digested peptides from purified D-xylonic acid dehydratase. Degenerate primers were designed according to the peptide sequences that were underlined. (2c) Partial DNA sequence and translated amino acid sequence of the D-xylonic acid dehydratase. The portion of underlined DNA labeled “3” encodes part of peptide 3; the portion of underlined DNA labeled “4” encodes peptide 4; and the portion of underlined DNA labeled “5” encodes part of peptide 5.

FIG. 3 illustrates the E. coli D-xylonic acid catabolic pathway, i.e. the pyruvate/glycolaldehyde pathway, and the genomic organization of its genes. (3a) Hypothetical E. coli D-xylonic acid catabolic pathway. Enzymes: (a) D-xylonic acid dehydratase; (b) 3-deoxy-D-glycero-pentulosonic acid aldolase. (3b and 3c) E. coli yjh and yag gene clusters.

FIG. 4 presents bar charts characterizing the performance of E. coli mutants grown on a single xylonate source. (4a) The growth character of E. coli strains on M9 medium containing D-xylonic acid as the sole carbon source. The plates were incubated at 37° C. for 72 h and the specific activity of D-xylonic acid dehydratase (open column) and 3-deoxy-D-glycero-pentulosonic acid aldolase (dotted column) of E. coli strains cultivated in LB medium containing D-xylonic acid. (4b) The catabolite accumulations of E. coli strains cultivated in LB medium containing D-xylonic acid (65 mM). D-xylonic acid (open column), 3-deoxy-D-glycero-pentulosonic acid (dotted column).

FIG. 5 presents a chart and graphs illustrating E. coli synthesis of 1,2,4-butanetriol from a xylose source, as well as a revised 1,2,4-butantriol biosynthesis pathway map. (5a) Summary of E. coli synthesis of 1,2,4-butanetriol in minimal salts mediums under fermentor-controlled cultivation conditions. (5b) Cell growth (open circles) and 1,2,4-butanetriol accumulation in the culture medium (hashed bars) by E. coli WN13/pWN7.126B. The arrows indicate the time points for D-xylose addition. (5c) Specific activities of D-xylose dehydrogenase (open column) and D-xylonic acid dehydratase (dotted column) during the cultivation of WN13/pWN7.126B. (5d) Revised D-1,2,4-butanetriol biosynthetic pathway map showing potential catabolic pathway steps that can divert carbon utilization from the main pathway to produce by-product compounds. Enzymes (with their genes) for labeled steps: (a) D-xylose dehydrogenase (xdh); (b) D-xylonic acid dehydratase (yjhG and yagF); (c) 2-keto acid decarboxylase (mdlC); (d) alcohol dehydrogenase (e.g., adhP); (e) 2-keto acid dehydrogenase (yiaE and ycdW); (f) 2-keto acid transaminase; (g) aldehyde dehydrogenase; (h) 3-deoxy-D-glycero-pentulosonic acid aldolase (yagE and yjhH); (k1) xylose isomerase; (k2) aldose reductase; (k3) xylonate dehydratase.

FIG. 6 presents a biosynthesis pathway map illustrating synthesis of byproducts of the common, D-1,2,4-butanetriol synthesis scheme, in a D-xylose-utilizing embodiment in which steps H and K1 are blocked. Exemplary net yields, from recombinant cell growth on minimal salts medium, for various compounds are shown, and enzyme identities for the depicted steps include: (a) D-xylose dehydrogenase (C. crescentus Xdh); (b) D-xylonate dehydratase (E. coli YjhG and YagF); (c) 2-keto acid decarboxylase (P. putida MdlC benzoylformate decarboxylase); (d) alcohol dehydrogenase (E. coli AdhP); (e) 2-keto acid dehydrogenase (E. coli KADH); (f) 2-keto acid transaminase (E. coli KAAT); (g) aldehyde dehydrogenase (E. coli ALDH); (h) 2-keto acid aldolase (E. coli YagE and YjhH; i.e. inactivated yagE and yjhH); and (k1) D-xylose isomerase (E. coli XylA; i.e. inactivated xylA).

FIG. 7 presents a schematic for the insertion of the adhP gene encoding alcohol dehydrogenase into the E. coli genome.

### DETAILED DESCRIPTION

The following description is merely exemplary in nature and is not intended to limit the present disclosure, application, or uses.

Subject matter of this application is related to subject matter of U.S. patent application Ser. No. 11/396,177, filed Mar. 31, 2006, International Patent Application No. PCT/US2004/031997, filed Sep. 30, 2004 and published Jul. 28, 2005 as WO 2005/068642, and U.S. Provisional Patent Application No. 60/507,708, filed Oct. 1, 2003, the disclosures of which are incorporated herein by reference.

The following definitions and non-limiting guidelines are to be considered in reviewing the description of this invention set forth herein. The headings (such as “Background” and “Summary,”) and sub-headings (such as “Screening Assays” and “Methods”) used herein are intended only for general organization of topics within the disclosure of the invention, and are not intended to limit the disclosure of the invention or any aspect thereof. The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features.

In particular, subject matter disclosed in the “Background” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., a “catalyst”) is made for convenience, and no inference should be drawn that the material must necessarily or solely function in accordance with its classification herein when it is used in any given composition. Specific Examples are provided for illustrative purposes of how to make and use the compositions and methods of this invention and, unless explicitly stated otherwise, are not intended to be a representation that given embodiments of this invention have, or have not, been made or tested.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

Unless otherwise indicated, articles such as “a” and “an” are used herein to indicate “at least one.” Terms such as having, including, containing, and comprising, used herein to describe a given embodiment, are open terms used to indicate that further components, e.g., ingredients, steps, or conditions, can be present in the embodiment.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As referred to herein, all compositional percentages are by weight of the total composition, unless otherwise specified.

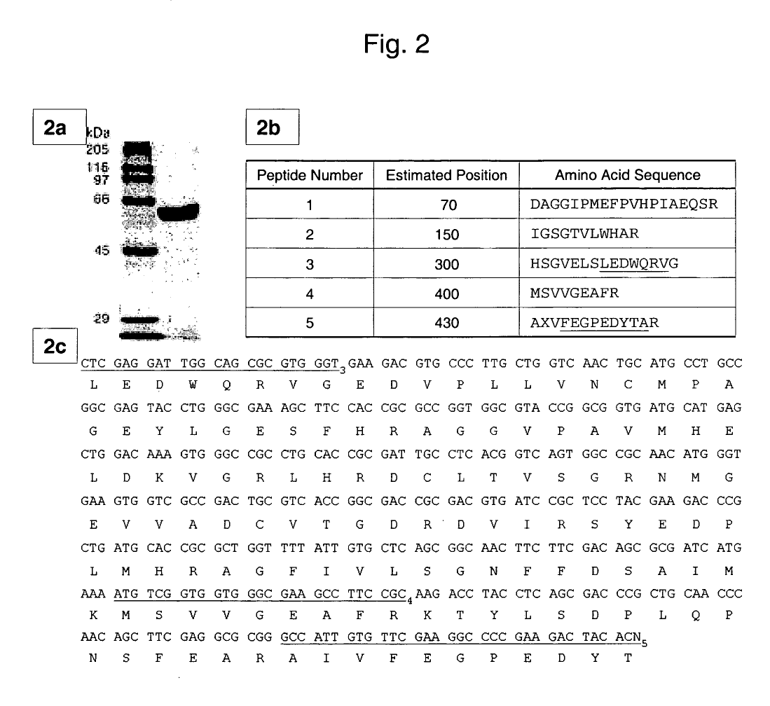

The present invention provides bioengineered synthesis methods, materials and organisms for producing D-1,2,4-butanetriol and intermediates from a carbon source. The bioconversion methods of the present invention are based on the de novo creation of biosynthetic pathways whereby D-1,2,4-butanetriol is synthesized from a carbon source (FIG. 2).

As used herein, members of a pair of acid-referent terms such as “xylonic acid” and “xylonate” are used interchangeably, unless otherwise indicated, either expressly or from context.

Antibodies, as used herein, include both native antibodies and recombinant antibodies, such as chimeric antibodies and CDR-grafted antibodies. As used herein, “antibody fragment” includes any polypeptides that contain an Fv structure identical in amino acid sequence to that of a whole antibody, whether native or recombinant, and which thereby retains binding specificity for the antigen or epitope for which the whole antibody is specific. Thus, antibody fragments, as used herein, include Fv, Fab, Fab′, F(ab′)₂, constant-domain-deleted antibodies (e.g., CH2-domain deleted antibodies), and single chain antibodies (e.g., scFv). Antibodies or antibody fragments can be monovalent or multivalent, i.e. the latter type having at least two Fv-type binding sites, at least one of which is an Fv structure having specificity for an enzyme polypeptide, nucleic acid, or nucleic acid analog hereof, or having specificity for such an Fv structure as does an anti-idiotypic antibody thereto.

As used herein, terms such as a “biocatalyst's gene,” refers to a nucleic acid that encodes the biocatalyst. Thus, reference to, e.g., a 3-deoxy-D-glycero-pentulosonic acid aldolase nucleic acid refers to a nucleic acid that encodes the specified aldolase. Biocatalysts, as used herein, can be traditional-polypeptide-type enzymes or antibody-based enzymes (abzymes) or can be nucleic acid-based enzymes (e.g., DNAzymes or RNAzymes).

As used herein, a “cellular entity” refers to a cell, or its protoplast or spheroplast, or a biocatalytically active cell fragment, e.g., a cytoplast, organelle, or lysate; where biocatalytic activity is retained after cell death, dead whole cell biocatalysts, e.g., cell ghosts, can be used. A cellular entity can comprise an organism, organ, tissue, tissue sample, cell culture, or other assemblage of cells. Microbial and plant cells can be particularly useful in some embodiments.

An extensive application of the thermally stable high energetic material, 1,2,4-butanetriol trinitrate, has been hindered by the lack of an economic route to synthesize its precursor, 1,2,4-butanetriol. In various embodiments, the present invention provides recombinant host cells that are capable of improved synthesis of D-1,2,4-butanetriol from D-xylose in minimal salts medium by following a previously established artificial biosynthetic pathway. Various embodiments of the present invention were made possible by the inventors' discovery of novel D-xylose dehydrogenases (Xdh), which can catalyze the oxidation of D-xylose into D-xylonic acid, and the elucidation of a previously unidentified D-xylonic acid catabolic pathway in wild-type Escherichia coli K-12.

In some embodiments hereof, a recombinant microbial host cell, e.g., a recombinant bacterial host cell, such as a recombinant E. coli is provided that can synthesize D-1,2,4-butanetriol directly from D-xylose in minimal salts medium. Thus, commercial scale biosynthetic production of D-1,2,4-butanetriol is now possible, and can permit, e.g., D-1,2,4-butanetriol trinitate to be more widely utilized. Experimental data indicates that D-1,2,4-butanetriol trinitrate exhibits the same explosive properties as racemic 1,2,4-butanetriol trinitrate, and thus D-1,2,4-butanetriol is equally useful a nitration target as racemic 1,2,4-butanetriol (J. Salan, Personal communication. Indian Head Division, Naval Surface Warfare Center, United States Navy. Indian Head, Md., 2005).

As noted above, major hurdles to further improvement of a D-xylose/xylonate-based biosynthetic approach to D-1,2,4-butanetriol production have included lack of genetic characterization of D-xylose dehydrogenases and the catabolic diversion of carbon from the biosynthetic pathway by an activity in the E. coli host strain. In various embodiments of the present invention, novel D-xylonic acid dehydratase enzymes, and their coding sequences, are now provided and characterized, such as the partial coding and amino acid sequences of the Pseudomonas fragi (ATCC 4973) D-xylonic acid dehydratase, and two newly discovered bacterial D-xylonic acid dehydratases. Novel D-xylonic acid dehydratase enzymes and genes from E. coli have also now been discovered.

In regard to the problem of catabolic diversion of carbon, various embodiments of the present invention provide enzymes, and their genes, from E. coli that catalyze such catabolism. Thus, in various embodiments, recombinant cells are now provided, in which such catabolic diversion is inhibited or inactivated. In various embodiments hereof, such cells are capable of biosynthesizing D-1,2,4-butanetriol in minimal salts medium. In various embodiments hereof, a recombinant cell is provided as a single cell that contains an enzyme system that is capable of D-xylose source-based D-1,2,4-butanetriol biosynthesis pathway. In some embodiments, recombinant D-1,2,4-butanetriol biosynthetic cells are provided that further have one or more knock-outs of the carbon-diverting catabolic activities.

In the proposed steps of the D-xylonate catabolic pathway, a dehydratase first catalyzes the conversion of D-xylonic acid into the 1,2,4-butanetriol pathway intermediate, 3-deoxy-D-glycero-pentulosonic acid, which is subsequently cleaved into pyruvate and glycolaldehyde via an aldolase-catalyzed reaction. Thus, elucidation of the D-xylonate catabolic pathway has resulted in identification of an aldolase-catalyzed pyruvate/glycolaldehyde biosynthetic activity that appears largely responsible for diversion of carbon from the 1,2,4-butanetriol biosynthesis pathway, with a concomitant decrease in yield.

An analysis using random transposon mutagenesis now reveals that the E. coli catabolism of D-xylonic acid is regulated through catabolite repression. Two sets of genes encoding the essential catabolic enzymes have now been identified in E. coli W3110 through use of enzyme assays and phenotype analysis of chromosomal knockout mutants. Genes yjhG and yagF (SEQ ID NOs:5 and 7) encode the D-xylonic acid dehydratases. Genes yjhH and yagE (SEQ ID NOs:11 and 13) respectively encode the corresponding 3-deoxy-D-glycero-pentulosonic acid aldolases.

In various embodiments, recombinant, D-xylose-to-D-1,2,4-butanetriol bioconverting cells (e.g., microbial cells; E. coli cells) are now provided in which 3-deoxy-D-glycero-pentulosonic acid aldolase activity is inhibited or inactivated, such as by disrupting the aldolase-encoding genes thereof. A cell that has been manipulated to inhibit or inactivate a 3-deoxy-D-glycero-pentulosonic acid aldolase polypeptide or nucleic acid thereof can be referred to herein as a recombinant DgPu⁻ cell.

In various embodiments hereof, D-xylose-to-D-1,2,4-butanetriol bioconverting E. coli cells have been manipulated to integrate an xdh gene into the chromosome thereof. In some embodiments thereof, such as in the exemplified E. coli WN13/pWN7.126B, greatly improved production of D-1,2,4-butanetriol has now been obtained, e.g., 6.2 g/L of D-1,2,4-butanetriol from D-xylose in 30% (mol/mol) yield under fermentor-controlled cultivation conditions. Other useful molecules that have now been identified in the culture medium include 3-deoxy-D-glycero-pentulosonic acid, 3-deoxy-D-glycero-pentanoic acid, (4S) 2-amino-4,5-dihydroxy pentanoic acid, and D-3,4-dihydroxy butanoic acid. Thus, enzyme systems and recombinant cells can also now be provided for biosynthesis of such other useful molecules.

Starting Materials for D-1,2,4-Butanetriol Biosynthesis. In various embodiments hereof, D-xylose can be used as a starting material for a D-1,2,4-butanetriol biosynthesis enzymatic pathway hereof. Various sources of D-xylose can be used. In some embodiments, a D-xylose source can be or comprise neat xylose or a mixture of xylose with other components. In some embodiments, a D-xylose source can be or comprise a non-xylose carbon source, wherein a recombinant cell that comprises an enzymatic pathway hereof, or that contains and is capable of expressing the genes thereof, is capable of utilizing the non-xylose carbon source to obtain D-xylose. Various such alternative xylose sources can be used. Thus, in some embodiments, a xylose source can comprise a simple carbon source, e.g., glucose, wherein the cell has the capability of synthesizing xylose therefrom. In some embodiments, a cell can have the capability of synthesizing xylose from a simple carbon source, such as glucose, by use of the cell's nucleotide sugars metabolism, starch or sucrose metabolism, or proteoglycan metabolism pathways. Various carbon sources can be used, based on a host cell's ability to convert it to D-xylose or D-xylonate. Some examples of simple carbon sources include C1 to C18 homo- or hetero-aliphatic compounds, including the C1-C8 heteroaliphatic compounds and carbon oxides, and host cell-hydrolyzable polymers containing residues thereof. In some embodiments, polyols or saccharides can be used.

In various embodiments, xylose can be synthesized from, e.g., glucose, by a cell comprising: (1) glucokinase (e.g., EC 2.7.1.1) to convert D-glucose to D-glucose-6-phosphate; (2) phosphoglucomutase (e.g., EC 5.4.2.2) to convert D-glucose-6-phosphate to D-glucose-1-phosphate; (3) UTP:glucose-1-phosphate uridylyltransferase (e.g., EC 2.7.7.91) to convert D-glucose-1-phosphate to UDP-D-glucose; (4) UDP-glucose 6-dehydrogenase (e.g., EC 1.1.1.22) to convert UDP-D-glucose to UDP-D-glucuronate; and (5) UDP-glucuronate decarboxylase (e.g., EC 4.1.1.35) to convert UDP-D-glucuronate to UDP-D-xylose. UDP-D-xylose can be hydrolyzed to provide D-xylose, or can be used to biosynthesize a xylose-residue-containing biopolymer, e.g., by action of a xylan synthase (e.g., EC 2.4.2.24), wherein the biopolymer can subsequently be hydrolyzed, e.g., as described below, to provide D-xylose. A cell useful in various embodiments hereof can have a native or recombinant ability to synthesize D-xylose from a simple carbon source. In some embodiments, a plant cell, or protoplast or spheroplast, can be used as a host cell that is capable of synthesizing D-xylose from a simple carbon source.

In some embodiments, a xylose source can be or comprise a xylose-residue-containing polymer, such as a xylose-residue-containing biopolymer, e.g., any xylose-residue-containing hemicellulose or pectin, wherein the cell has the capability of synthesizing xylose therefrom Thus, a xylose source can be or comprise any one or more of: the homo- or hetero-xylans, e.g., glucuronoxylans, arabino-glucuronoxylans, arabinoxylans, or glucurono-arabinoxylans; the xyloglucans; the xylogalacturonans; the xylogalactans; the xylofucans or xylogalactofucans; and the like; or any combination of thereof. A cell having the capability of synthesizing xylose from a xylose-residue-containing polymer can comprise enzymes providing that capability, such as a xylanase (e.g., EC 3.2.1.8; 3.2.1.32; 3.2.1.126; 3.2.1.136; or 3.2.1.156) for hydrolyzing homo- or hetero-xylan backbone xylose residue bonds, and/or a xylosidase (e.g., EC 3.2.1.32; 3.2.1.37; or 3.2.1.72) for hydrolyzing pendant xylose residue bonds. The xylanase(s) and/or xylosidase(s) can be present either alone or in combination with other, non-xylanase/non-xylosidase, polymer-operative or polymer fragment-operative hydrolytic enzyme(s), such as one or more of: a glycosidase; an esterase; a glycuronosidase; a glycanase, e.g., an exo- or endo-glucanase or -galactanase or -fucanase; a glycuronidase, e.g., an exo- or endo-galacturonase; or a combination thereof. A cell useful in various embodiments hereof can have a native or recombinant ability to synthesize D-xylose from a xylose-residue-containing polymer.

In some embodiments, a xylose source can comprise D-xylulose or D-xylitol, wherein the cell has the capability of synthesizing xylose therefrom, such as wherein the cell comprises a xylose isomerase (EC 5.3.1.5) or aldose reductase (EC 1.1.1.21), respectively. A cell useful in various embodiments hereof can have a native or recombinant ability to synthesize D-xylose from D-xylulose or D-xylitol

In various embodiments, D-xylonic acid can be used as a starting material for a 1,2,4-butanetriol biosynthesis enzymatic pathway hereof. Various sources of D-xylonic acid can be used. In some embodiments, a D-xylonate source can be or comprise neat D-xylonic acid or a mixture of xylonic acid with other components. In some embodiments, a D-xylonate source can be or comprise a non-xylonate carbon source, wherein a recombinant cell that comprises an enzymatic pathway hereof, or that contains and is capable of expressing the genes thereof, is capable of utilizing the non-xylonate carbon source to obtain D-xylonic acid. Various such alternative xylonic acid sources can be used. Thus, in some embodiments, a xylonate source can comprise a simple carbon source, e.g., glucose, wherein the cell has the capability of synthesizing xylonate therefrom. In some embodiments, a xylonate source can comprise 2-dehydro-3-deoxy-D-xylonate, wherein the cell has the capability of synthesizing xylonate therefrom, such as wherein the cell comprises a xylonate dehydratase (EC 4.2.1.82). A cell useful in various embodiments hereof can have a native or recombinant ability to synthesize D-xylose from, e.g., a simple carbon source, or from 2-dehydro-3-deoxy-D-xylonate.

In various embodiments, a xylose source or a xylonate source for use herein can comprise D-xylonolactone, wherein the cell is capable of converting it to xylose or xylonate, respectively; such as wherein the cell comprises a D-xylose-1-dehydrogenase (EC 1.1.1.175) or a xylono-1,4-lactonase (EC 3.1.1.68), respectively. A cell useful in various embodiments hereof can have a native or recombinant ability to synthesize D-xylose or D-xylonate from D-xylonolactone.

The capability to utilize a xylose source or xylonate source can be native to the cell used to prepare a recombinant cell hereof, or can be recombinantly added to the cell. Examples of cells having a native capability for converting xylose-residue-containing biopolymers to D-xylose include fungal cells, such as Neurospora, Aspergillus, and Penicillium, and bacterial cells, such as Bacillus, Pseudomonas, and Streptomyces. However, a recombinant 1,2,4-butanetriol synthesizing cell hereof can be co-cultured, in the presence of a xylose-residue-containing biopolymers, with a cell having a native or recombinant capability for converting xylose-residue-containing polymers to D-xylose, such as a cell that secretes hemicellulase(s), to provide xylose to the recombinant 1,2,4-butanetriol synthesizing cell. Similar co-culturing can be done, where another alternative xylose source or xylonate source is used, with a cell having the ability to secrete enzymes that perform the conversion to xylose or xylonate.

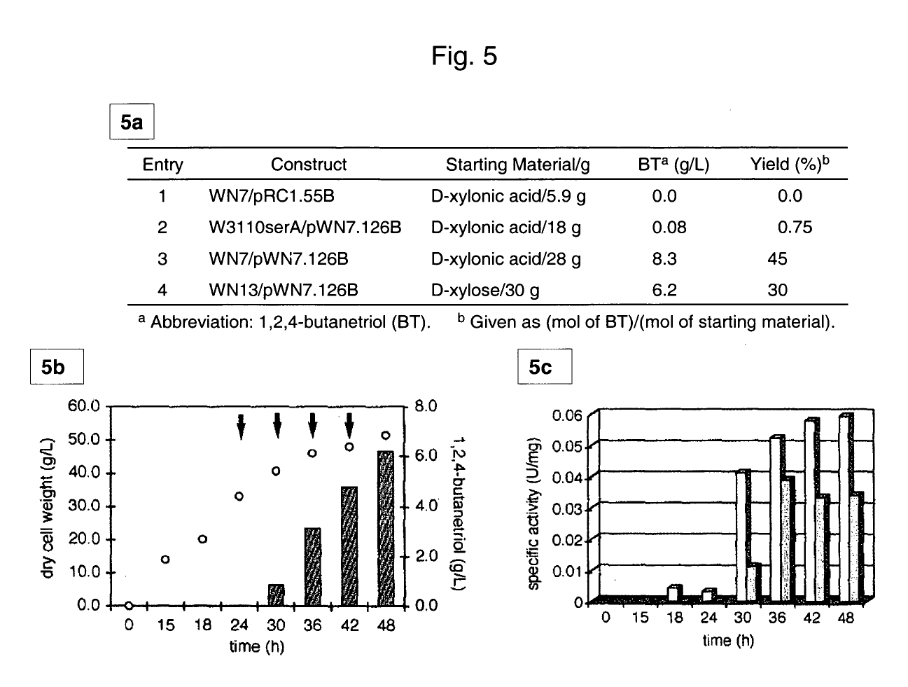

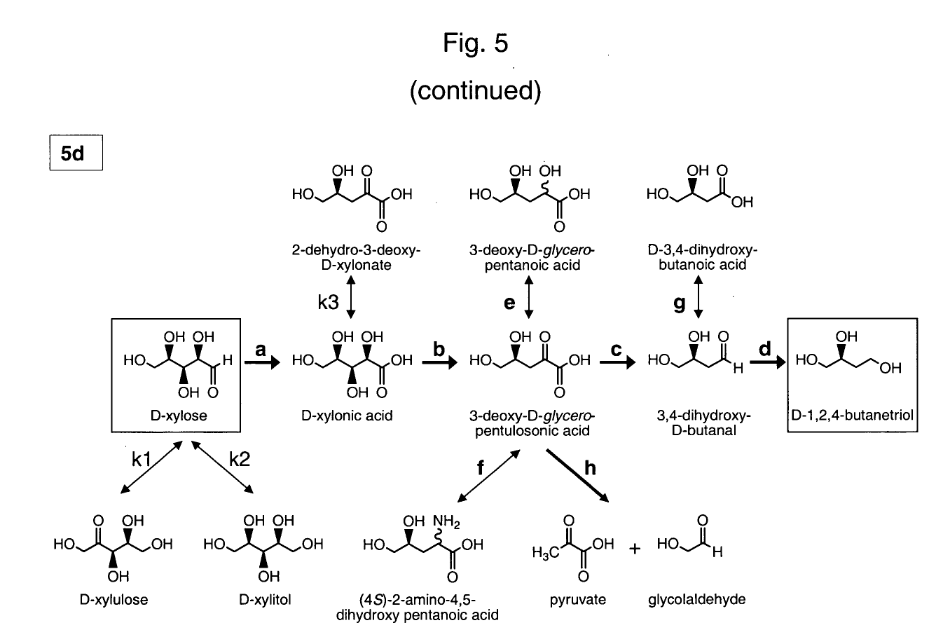

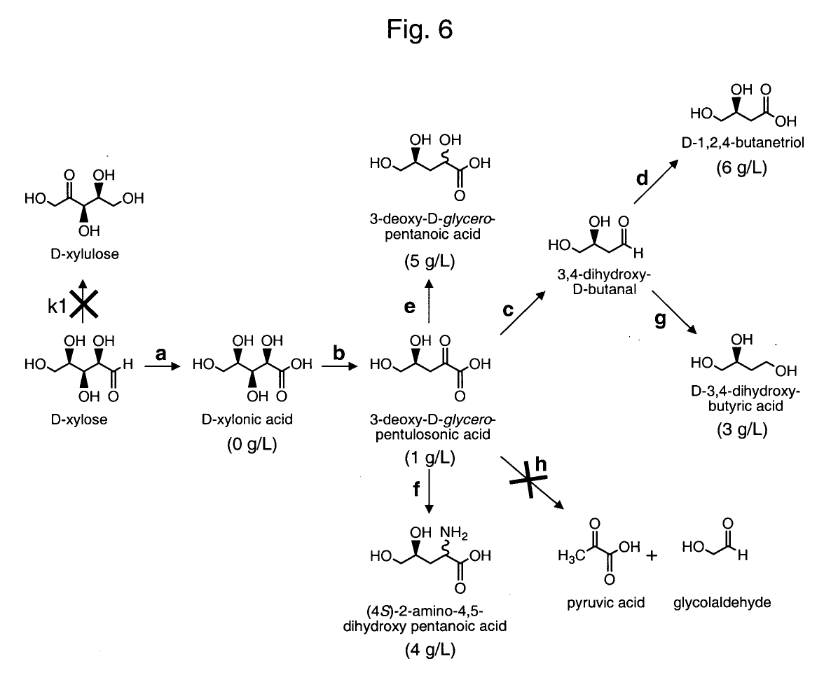

Biosynthetic Pathways for D-1,2,4-Butanetriol Production. Referring to FIG. 5d, in various embodiments, a D-1,2,4-butanetriol biosynthetic pathway hereof can utilize steps B, C, and D of FIG. 5d, using a xylonate source, or steps A, B, C, and D, using a xylose source. These steps are catalyzed by: (a) a D-xylose dehydrogenase (xdh), (b) a D-xylonic acid dehydratase (e.g., yjhG or yagF); (c) a 2-keto acid decarboxylase (e.g., mdlC); and (d) an alcohol dehydrogenase. As used herein, in the context of such a step (d), “alcohol dehydrogenase” refers to any alcohol dehydrogenase enzyme having a catalytic activity that converts 3,4-dihydroxy-D-butanal to D-1,2,4-butanetriol, e.g., an AdhP, or an AdhE or YiaY, type of alcohol dehydrogenase. In the examples hereof, a Pseudomonas putida md/C coding sequence encoding benzoylformate decarboxylase (EC 4.1.1.7) is used to provide the 2-keto acid decarboxylase activity. Enzymes for steps A and B are described in more detailed in subsequence sections.

In the examples hereof, native E. coli dehydrogenase activity is used to catalyze the final step (d) of the formation of 1,2,4-butanetriol. Although not wishing to be bound by theory, it is believed that this dehydrogenase activity is effected by one or more primary alcohol dehydrogenases; these are also known as aldehyde reductases. However, any enzymes exhibiting such an aldehyde reductase activity, i.e. that is capable of reducing 3,4,-dihydroxybutanal to 1,2,4-butanetriol, may be substituted. Examples of other enzymes exhibiting useful aldehyde reductase activities include, e.g., primary alcohol dehydrogenases not native to E. coli, or not native to the host cell in an in vivo embodiment hereof, and carbonyl reductases. Specific examples of these include NADH-dependent alcohol dehydrogenases (EC 1.1.1.1), NADPH-dependent alcohol dehydrogenases (EC 1.1.1.2), and NADPH-dependent carbonyl reductases (EC 1.1.1.184).

An enzyme system that is operative to effect a biocatalytic pathway hereof can be provided by inserting at least one gene into a selected host cell, to construct a pathway not present in the wild type cell. Thus, a recombinant host cell capable of 1,2,4-butanetriol production according to an in vivo embodiment of the present invention is one that has been transformed so as to become capable of at least one of: producing D-1,2,4-butanetriol from D-xylose or producing D-1,2,4-butanetriol from D-xylonic acid.

Methods and systems for biosynthesis of D-1,2,4-butanetriol according to the present invention can be operated either with or without the presence of a method or system for biosynthesis of L-1,2,4-butanetriol. In embodiments in which both D- and L-1,2,4-butanetriols are synthesized concurrently, a resulting mixture of isomers can be nitrated to form D,L-1,2,4-butanetriol trinitrate.

1,2,4-Butanetriol Uses and Derivatives. 1,2,4-Butanetriol prepared according to an embodiment of the present invention can be isolated, e.g., for use as, e.g., a serum glycerides chromatography standard (see, e.g., H. Li et al., J Lipid Res. (Jun. 20, 2006) [Epub ahead of print at the http World-Wide-Website jlr.org/cgi/reprint/D600009-JLR200v1]), and/or the 1,2,4-butanetriol can be derivatized to form desired product(s).

In various embodiments, 1,2,4-butanetriol trinitrate can be produced as the derivative by nitration. Nitration of 1,2,4-butanetriol produced in an embodiment hereof can be readily performed by use of a variety of commercially available nitrating agents. Common nitrating agents include: HNO₃ (or mixtures of HNO₃ and H₂SO₄), N₂O₄ (or mixtures of N₂O₄ and NO₂), N₂O₅ (or mixtures of N₂O₅ and HNO₃), NO₂Cl, peroxynitrite salts (X⁺ O═N—O—O⁻, commercially available as, e.g., Na⁺, K⁺, Li⁺, ammonium, or tetraalkylammonium peroxynitrites), and tetranitromethane, and compositions containing one or more such agent. These may be used according to any of the various nitration conditions and procedures known in the art to obtain 1,2,4-butanetriol trinitrate.

Alternatively, 1,2,4-butanetriol produced in an embodiment hereof can be converted to other useful derivative compounds whether by a biosynthetic or chemosynthetic route; see, e.g., N. Shimizu et al., Biosci. Biotechnol. Biochem. 67(8):1732-1736 (August 2003).

As described later herein, fermentor cultivation may be used to facilitate conversion of the carbon source to D-1,2,4-butanetriol. The culture broth may then be nitrated to form the butanetriol-trinitrate from the culture broth. In another embodiment, the butanetriol may be extracted from the culture broth, washed or purified and subsequently nitrated. The fed-batch fermentor process, precipitation methods and purification methods are known to those skilled in the art.

Once formed, the 1,2,4-butanetriol trinitrate can be used as an active ingredient in an energetic (e.g., explosive) composition, which can be in the form of an explosive device or a, e.g., rocket, fuel. Explosive devices include those designed for use in or as munitions, quarrying, mining, fastening (nailing, riveting), metal welding, demolition, underwater blasting, and fireworks devices; the devices may also be designed or used for other purposes, such as ice-blasting, tree root-blasting, metal shaping, and so forth.

In forming an energetic (e.g., explosive) composition, the 1,2,4-butanetriol trinitrate can be mixed with a further explosive compound, and, alternatively or in addition, with a non-explosive component, such as an inert material, a stabilizer, a plasticizer, or a fuel. Examples of further explosive compounds include, but are not limited to: nitrocellulose, nitrostarch, nitrosugars, nitroglycerin, trinitrotoluene, ammonium nitrate, potassium nitrate, sodium nitrate, trinitrophenylmethylnitramine, pentaerythritol-tetranitrate, cyclotrimethylene-trinitramine, cyclotetramethylene-tetranitramine, mannitol hexanitrate, ammonium picrate, heavy metal azides, and heavy metal fulminates. Further non-explosive components include, but are not limited to: aluminum, fuel oils, waxes, fatty acids, charcoal, graphite, petroleum jelly, sodium chloride, calcium carbonate, silica, and sulfur.

Thus, compositions containing 1,2,4-butanetriol trinitrate produced by a process hereof and explosive devices containing such 1,2,4-butanetriol trinitrate can also now be provided. 1,2,4-Butanetriol trinitrate prepared by a process according to an embodiment of the present invention can be used in a methods for blasting or propelling a material object comprising detonating, at a position upon, or adjacent to, a surface of said material object, an explosive device containing such 1,2,4-butanetriol trinitrate.

Other articles and compositions according to embodiments hereof include the following. Recombinant host cells containing an enzyme system according to an embodiment hereof, and such cells that are DgPu⁻ cells. DgPu⁻ cells. Recombinant host cells containing expressible nucleic acid encoding an enzyme system according to an embodiment hereof. Kits comprising a composition containing such an enzyme system, with instructions for the use thereof for the production 1,2,4-butanetriol or other desired product; kits comprising nucleic acid encoding such an enzyme system, with instructions for the use thereof for the formation of a recombinant cell capable of producing 1,2,4-butanetriol or other desired product; kits comprising a composition containing recombinant host cells capable of expressing such an enzyme system, with instructions for the use thereof for the production 1,2,4-butanetriol or other desired product.

Alternative Biosynthetic Products, Other than Butanetriol, and Pathways Therefor. As part of the work leading to the present invention, a number of previously unrecognized by-products of the 1,2,4-butanetriol-biosynthetic pathway were identified in 1,2,4-butanetriol-synthesizing cells according to the present invention that had their pyruvate/glycolaldehyde catabolic pathway (FIG. 5d Step h) blocked by inactivation of their 3-deoxy-D-glycero-pentulosonic acid aldolases genes. Among these by-product compounds are: (1) 3-deoxy-D-glycero-pentanoic acid, formed from 3-deoxy-D-glycero-pentulosonic acid by action of a 2-keto-acid reductase activity; (2) D-3,4-dihydroxy-butanoic acid, formed from 3,4-dihydroxy-D-butanal by action of an aldehyde dehydrogenase activity; and (3) (4S)-2-amino-4,5-dihydroxy pentanoic acid, formed from 3-deoxy-D-glycero-pentulosonic acid by action of a 2-keto acid transaminase activity.

These compounds contain chiral centers and so can be useful in the synthesis of bioactive and other agents, such as those of the following examples. 3-Deoxy-D-glycero-pentanoic acid can be used to prepare 3-deoxy pentanoic acid lactone, a feeding promoter compound that can be added as a growth promoter in livestock feed; see, e.g., U.S. Pat. No. 5,391,769, Matsumoto et al., issued Feb. 21, 1995. 3,4-Dihydroxy-butanoic acid can be used to synthesize anti-hypercholesterolemic agents; see, e.g., U.S. Patent Publication 2006/0040898, Puthiaparampil et al, published Feb. 23, 2006 and U.S. Pat. No. 5,998,633, Jacks et al., issued Dec. 7, 1999. 2-Amino-4,5-dihydroxypentanoic acid can be used to form metalloproteinase inhibitor compounds; see, e.g., D. T. Elmore, “Peptide Synthesis,” chap. 1 in Amino Acids, Peptides and Proteins, vol. 34, (RSC, 2003) (at p. 18).

Thus, in various embodiments, a 3-deoxy-D-glycero-pentanoic acid biosynthetic pathway hereof can utilize steps B and E of FIG. 5D, using a xylonate source, or steps A, B, and E, using a xylose source. In various embodiments, a D-3,4-dihydroxy-butanoic acid biosynthetic pathway hereof can utilize steps B, C, and G of FIG. 5D, using a xylonate source, or steps A, B, C, and G, using a xylose source. In various embodiments, a (4S)-2-amino-4,5-dihydroxy pentanoic acid biosynthetic pathway hereof can utilize steps B and F of FIG. 5D, using a xylonate source, or steps A, B, and F, using a xylose source. In some embodiments of any one of these, one or more of the post-Step B enzyme(s) that catalyze(s) the alternative conversion to one of the other two compounds, can be inhibited or inactivated, and, optionally, one or more of the enzyme(s) that catalyze(s) the conversion to 1,2,4-butanetriol, and/or the 3-deoxy-D-glycero-pentulosonic acid aldolase(s), can be inhibited or inactivated, as can one or more of any other enzyme(s) that divert xylose, xylonate, or other intermediates of the selected pathway(s) from use therein. Similarly, one or more of enzyme(s) catalyzing steps E, F, and/or G can be inhibited or inactivated in various embodiments of enzyme systems hereof capable of synthesizing 1,2,4-butanetriol.

Inactivation or Inhibition of Undesirable Catabolic Activity. In various embodiments in which a xylose source other than D-xylitol is used in a xylose-bioconverting pathway hereof, a host cell's aldose reductase(s) of FIG. 5D Step k2, and/or an enzyme(s) acting on the xylitol product thereof, can be inhibited or inactivated to prevent diversion of xylose; similarly, where such a xylose source other than D-xylulose is used therein, a host cell's xylose isomerase(s) of FIG. 5D Step k1, and/or an enzyme(s) acting on the D-xylulose product thereof, such as xylulokinase(s) (EC 2.7.1.17), can be inhibited or inactivated to help prevent diversion of xylose. Where a xylose source comprises, e.g., xylose, a xylose-residue-containing polymer, or a simple carbon source, both such strategies can be employed together to help prevent xylose diversion.

In various embodiments in which a xylonate source other than D-xylonic acid is used in a xylonate-bioconverting pathway hereof, or in various embodiments in which a xylose-bioconverting pathway is employed, a host cell's xylonate dehydratase(s) of FIG. 5D Step k3, and/or an enzyme(s) acting on the 2-dehydro-3-deoxy-D-xylonate product thereof, e.g., 2-dehydro-3-deoxy-D-pentonate aldolase (EC 4.1.2.28) of FIG. 5D Step h, can be inhibited or inactivated to help prevent diversion of xylonate. Therefore, any or all pathways that divert a desired starting material or intermediate from a selected biosynthetic pathway according to an embodiment of the present invention, can be inhibited or inactivated.

In any biosynthetic pathways hereof, whether utilizing a xylose or xylonate source, an enzyme(s) acting on the 2-dehydro-3-deoxy-D-xylonate product thereof, e.g., 2-dehydro-3-deoxy-D-pentonate aldolase (EC 4.1.2.28) of FIG. 5D Step h, can be inhibited or inactivated to help prevent diversion of carbon from the desired pathway.

With reference to FIG. 5d, as noted above, Step E is catalyzed by a 2-ketoacid reductase activity (or alpha-hydroxyacid dehydrogenase; e.g., EC 1.1.99.6), one 2-ketoacid reductase sequence being, e.g., Genbank Accession No. AAC74117.gi:87081824, encoded by U00096 . . . gi:48994873. Step F is catalyzed by a 2-ketoacid-operative transaminase activity (e.g., EC 2.6.1.21 or 2.6.1.67), one transaminase sequence being, e.g., Genbank Accession No. YP_{—}556835.gi:91781629, encoded by nt280347-281312 of NC_{—}007951.gi:91781384. Step G is catalyzed by an aldehyde dehydrogenase activity (e.g., EC 1.2.1.3; 1.2.1.4; 1.2.1.5; 1.2.99.3; or 1.2.99.7), one aldehyde dehydrogenase sequence being, e.g., Genbank Accession No. AAA23428.gi:145224, encoded by M38433.gi:145223. Step K1 is catalyzed by xylose isomerase (EC 5.3.1.5), one xylose isomerase sequence being Genbank Accession No. ABG71642.gi:110345405, encoded by CP000247.gi:110341805. Step K2 is catalyzed by aldose reductase (EC 1.1.1.21), one aldose reductase sequence being Genbank Accession No. AAG54503.gi:12512935, encoded by AE005174.gi:56384585. Step K3 is catalyzed by xylonate dehydratase (EC 4.2.1.82); see, e.g., AS Dahms & A Donald, “D-xylo-Aldonate dehydratase,” Methods Enzymol. 90(Pt. E):302-305 (1982).

FIG. 5d Step H is catalyzed by a 3-deoxy-D-glycero-pentulosonic acid aldolase, sequences of which include SEQ ID NOs:12 and 14, encoded by SEQ ID NOs:11 and 13, respectively. These sequences can be used, e.g., by bioinformatic searching or hybridization assays, to identify other such undesirable, 3-deoxy-D-glycero-pentulosonic acid aldolase genes in cells targeted for development into a recombinant host cell according to an embodiment hereof. These gene sequences, and the gene sequences of such other catabolic aldolases identified by use thereof, can be used to construct polynucleotide vectors, e.g., plasmids, designed to inactivate such aldolase genes. RNA interference techniques can alternatively be used to inhibit expression of such genes. Thus, 3-deoxy-D-glycero-pentulosonic acid aldolase activities can be inhibited or inactivated in a desired host cell.

Thus, also provided herein are novel enzyme systems, and recombinant cells solely or jointly comprising enzymes systems, for synthesis of one or more of D-1,2,4-butanetriol, 3-deoxy-D-glycero-pentanoic acid; D-3,4-dihydroxy-butanoic acid; or (4S)-2-amino-4,5-dihydroxy pentanoic acid. In various embodiments, such enzyme systems or recombinant cells are capable of synthesizing the compound(s) from a xylose source or xylonate source.

3-deoxy-D-glycero-pentulosonic acid aldolase can also be inhibited or inactivated in recombinant host cells containing an engineered biopathway for L-1,2,4-butanetriol biosynthesis from L-arabinose or L-arabinonic acid, to similarly prevent diversion of 3-deoxy-D-glycero-pentulosonate therefrom. A cellular entity that has been manipulated to inhibit or inactivate a 3-deoxy-D-glycero-pentulosonic acid aldolase polypeptide or nucleic acid thereof can be referred to herein as a recombinant DgPu⁻ entity e.g., a recombinant DgPu⁻ cell.

Enzyme Polypeptides and Coding Sequences. In some embodiments according to the present invention, a polypeptide is provided that has D-xylose dehydrogenase activity. Each of SEQ ID NOs:2 and 4 presents the amino acid sequence of a wild-type xylose dehydrogenase (Xdh) that acts to catalyze the conversion of D-xylose to D-xylonate. In some embodiments according to the present invention, a polynucleotide, or nucleic acid analog, is provided that encodes a D-xylose dehydrogenase enzyme hereof. Each of SEQ ID NOs:1 and 3 presents the DNA coding sequence of a wild-type D-xylose dehydrogenase (xdh).

In some embodiments according to the present invention, a polypeptide is provided that has D-xylonate dehydratase activity. Each of SEQ ID NOs:6 and 8 presents the amino acid sequence of a wild-type D-xylonate dehydratase that acts to catalyze the conversion of D-xylonate to 3-deoxy-D-glycero-pentulosonate: E. coli YjhG and YagF. In some embodiments according to the present invention, a polynucleotide, or nucleic acid analog, is provided that encodes a D-xylonate dehydratase enzyme hereof. Each of SEQ ID NOs:5 and 7 presents the DNA coding sequence of a wild-type D-xylonate dehydratase: E. coli yjhG and yagF.

Similarly, SEQ ID NO:10, encoded by SEQ ID NO:9, presents that amino acid sequence of a This P. fragi D-xylonic acid dehydratase fragment from Pseudomonas fragi., which bacterium is publicly available from the American Type Culture Collection (Manassas, Va., U.S.) under Accession No. ATCC 4973. This D-xylonate dehydratase, and its gene, can be isolated from the bacterium using any of the techniques known in the art, e.g., those described in the Examples section below. The DNA coding sequence of this enzyme has a putative length of about 1300 nt, and has a 3′-terminal portion comprising the base sequence of SEQ ID NO:9 near its end. The encoded D-xylonate dehydratase polypeptide has a putative length of about 430+ residues, an approximate MW of about 60 kDa, and has a C-terminal portion comprising the amino acid sequence of SEQ ID NO:10 near its end. This enzyme is also capable of catalyzing the conversion of D-xylonic acid to 3-deoxy-D-glycero-pentulosonic acid.

In some embodiments according to the present invention, a polynucleotide is provided that encodes, or that contains coding sequence from, a 3-deoxy-D-glycero-pentulosonate aldolase. Each of SEQ ID NOs:12 and 14 presents the amino acid sequence of a wild-type aldolase that can catalyze the conversion of 3-deoxy-D-glycero-pentulosonate to pyruvate and glycolaldehyde: E. coli YjhH and YagE. Nucleic acid sequences encoding these amino acid sequences can be used, as described above, to construct knock-out vectors or RNA interference vectors. In some embodiments according to the present invention, a polynucleotide, or nucleic acid analog, is provided that encodes a D-xylonate dehydratase enzyme hereof, e.g., each of SEQ ID NOs:11 and 13 presents the DNA coding sequence of a wild-type 3-deoxy-D-glycero-pentulosonate aldolase: E. coli yjhH and yagE.

Likewise, residues 19-319 of SEQ ID NO:12 present the alternative amino acid sequence of the wild-type E. coli YjhH aldolase that can catalyze the conversion of 3-deoxy-D-glycero-pentulosonate to pyruvate and glycolaldehyde. Nucleic acid sequences encoding this amino acid sequence can be used, as described above, to construct knock-out vectors or RNA interference vectors. In some embodiments according to the present invention, a polynucleotide, or nucleic acid analog, is provided that encodes a D-xylonate dehydratase enzyme hereof, e.g., nt 55-957 of SEQ ID NOs:11 present the DNA coding sequence of the alternative amino acid sequence of the wild-type E. coli YjhH aldolase. The full or the alternative nucleotide sequence of SEQ ID NO:11 can be used, e.g., to screen for other such aldolases and/or to prepare knock-out or RNA interference vectors. The full or alternative amino acid sequence of SEQ ID NO:12 can be used, e.g., to catalyze the stated reaction or as an epitopic target for antibody and binding molecule production and/or selection.

Enzyme-Encoding Nucleic Acid and Polypeptide Variants. A coding sequence according to the present invention can be operably attached to transcription and/or translation control elements that are functional in a desired host cell, such as a microbial (e.g., bacteria, fungi/yeast, archaea, or protist) or plant (e.g., dicot, monocot, gymnosperm, bryophyte, or pteridophyte) cell, although a vertebrate (e.g., mammalian animal or human) or invertebrate (e.g., insect) cell can be used. Nucleic acids hereof can be incorporated into nucleic acid vectors and/or can be used to transform host cells. Examples of genetic elements, vectors, and transformation techniques include those described in U.S. Pat. Nos. 6,803,501, Baerson et al., issued Oct. 12, 2004, and 7,041,805, Baker et al., issued May 9, 2006, the descriptions thereof being incorporated herein by reference.

Coding sequences hereof can be mutated, e.g., as by random or directed mutation, to introduction amino acid substitutions, deletions, or insertions; conservative amino acid substitutions may be introduced thereby. Useful conservative amino acid substitutions include those described, e.g., in U.S. Pat. No. 7,008,924, Yan et al., issued Mar. 7, 2006 the description thereof being incorporated herein by reference. Hybridization under conditions of stringency, or manual or automated (e.g., bioinformatic) sequence comparison, may be performed, using the sequence of a polypeptide or nucleic acid hereof, to screen for further candidate enzyme polypeptides or further candidate enzyme-encoding polynucleotides, e.g., homologous polypeptide and polynucleotides, having or encoding a biocatalytic activity that is the same as that of an enzyme defined herein with reference to a sequence in the Sequence Listing. Useful measures of sequence homology (similarly and identically of aligned sequences) and stringent hybridization conditions for hybridization screening include those described, e.g., in U.S. Pat. Nos. 7,049,488, Fischer et al., issued May 23, 2006, and 7,041,805, Baker et al., issued May 9, 2006, the descriptions thereof being incorporated herein by reference. In some embodiments, a homologous amino acid sequence can be at least 70%, or about or at least 75%, 80%, 85%, 90%, or 95% homologous to that a given Sequence Listing-listed polypeptide. In some embodiments, a homologous nucleobase sequence can be about or at least 90%, or 95%, 98% homologous to that of a given Sequence Listing-listed polynucleotide. A coding sequence according to the present invention can be codon-optimized to improve expression in a desired host cell, according to any of the techniques known in the art, e.g., as described in U.S. Pat. No. 6,858,422, Giver et al., issued Feb. 22, 2005, the description thereof being incorporated herein by reference. Thus, conservative-substituted amino acid variants of a given enzyme hereof and homologous enzymes to a given enzyme hereof, retaining the same type of biocatalytic activity, can be used for the same function in enzyme systems, pathways, and methods hereof.

Polynucleotides according to the present invention, e.g., polynucleotides comprising a base sequence of any one of SEQ ID NOs:1, 3, 5, 7, 9, 11, or 13, and other same-activity-enzyme-encoding polynucleotides hereof, can be used as templates in a directed evolution process employed to obtain a desired enhancement or variation in function of the respective encoded enzyme, e.g., by two or more rounds of gene recombination (e.g., gene shuffling), and/or random mutation (e.g., by error-prone PCR) or directed mutation (e.g., point mutation) to the template(s). Coding sequences, and genes of which they form an operative part, can be codon optimized to function, or to function better, in a selected host cell. Any of the many codon-optimization techniques known in the art can be used.

Basic DNA manipulations and genetic techniques useful herein can be performed according to standard protocols as described, e.g., in T. Maniatis et al. Molecular Cloning: A Laboratory Manual (Cold Spring Harbor Laboratory Press, Cold Spring Harbor, N.Y., 1982); and J. Sambrook et al., Molecular cloning: A laboratory manual (2nd ed., Cold Spring Harbor Laboratory Press, Cold Spring Harbor, N.Y., 1989), incorporated herein by reference.

Screening Assays. In some embodiments of the present invention, an enzyme polypeptide-encoding nucleic acid or nucleic acid analog hereof can be used to screen a sample at least suspected of containing another same-activity-enzyme-encoding nucleic acid, by a duplex- or triplex-forming hybridization assay. A probe useful for this purpose can comprise a contiguous base sequence of at least 10, or about or at least 20, 30, 40, or 50 bases from the polypeptide-encoding nucleic acid hereof. The probe(s) can be detectably labeled, e.g., with a colored, unquenched or reversibly quenched fluorescent, luminescent, or phosphorescent label, or a label that can be reacted to produce a detectable signal, such as a photonic signal, or a binding site- or binding molecule-type label, such as a biotin- or avidin-labeled probe that can be reacted to attach a moiety that provides a detectable signal. Similarly, the nucleobase sequence information of an enzyme polypeptide-encoding nucleic acid can be used in a bioinformatic method, e.g., in silico or by direct visualization, to identify another nucleobase sequence as a, or as a candidate, same-activity-enzyme-encoding sequence.

Antibodies can be prepared that have binding specificity for an enzyme polypeptide or nucleic acid according to various embodiments hereof. Such antibodies can be used to screen biomolecule libraries, mixtures, and so forth that are at least suspected of containing a same-activity enzyme or same-activity-enzyme-encoding nucleic acid, i.e. the activity being of the same type as the biomolecule providing the sequence or serving as the antigen. Anti-idiotypic antibodies to such antibodies can also be prepared and used for screening purposes. The antibodies can be detectably labeled. Aptamers having such binding specificity can alternatively be prepared and used for this purpose.

### EXAMPLES

Isolation of a partial gene sequence of the Pseudomonas fragi (ATCC 4973) D-xylonic acid dehydratase. The D-xylose catabolic pathway in Pseudomonas fragi (ATCC 4973) is induced when this carbohydrate is available as a carbon source for growth. See, e.g., R. Weimberg, Pentose oxidation by Pseudomonas fragi, J. Biol. Chem. 236:629-635 (1961). Therefore, the D-xylonic acid dehydratase was purified from cells cultivated in medium containing D-xylose. The purification was performed using a DE-52 anion exchange column, a hydroxyapatite column, a phenylsepharose column, and an HPLC Resource anion exchange column. This method resulted in a 97-fold purification with protein purity of near homogeneity based on an SDS-PAGE analysis. The molecular weight of the purified protein was estimated to be 60 kDa on a denaturing protein gel (FIG. 2a).

To isolate the gene that encodes the purified D-xylonic acid dehydratase, the protein was processed by trypsin digestion and N-terminal sequence analysis of the HPLC-purified digestion products. Amino acid sequences of five short peptides were thus obtained (FIG. 2b). A BLAST analysis of the NCBI database for short and nearly exact matches of the five peptide sequences revealed several proteins that contained amino acid sequences with close to 80% homology to all the five queries. The relative positions of the five peptides in P. fragi D-xylonic acid dehydratase were therefore estimated using the relative positions of their homologs in the parent proteins from the NCBI database. Using a pair of degenerate primers that was designed according to the partial amino acid sequences of peptide 3 and peptide 5, we successfully amplified a single DNA product from the genomic DNA of P. fragi. The PCR product was cloned into pCRTOPO-2.1 vector and the DNA sequence of the insert was determined (FIG. 2c). To further evaluate whether this 410 by DNA fragment encoded the purified D-xylonic acid dehydratase, we examined the peptide sequence that was translated from the DNA sequence “in frame” (FIG. 2c).

The N-terminus of the peptide contained the partial amino acid sequence of peptide 3 stretching to its C-terminal end (FIG. 2c). The C-terminus of the peptide contained the partial amino acid sequence of peptide 5 stretching to its N-terminal end (FIG. 2c). Furthermore, the translated peptide also contained the entire amino acid sequence of peptide 4, which was estimated to be situated between peptide 3 and peptide 5 in D-xylonic acid dehydratase. We therefore concluded that the PCR product is a partial gene encoding the D-xylonic acid dehydratase from P. fragi.

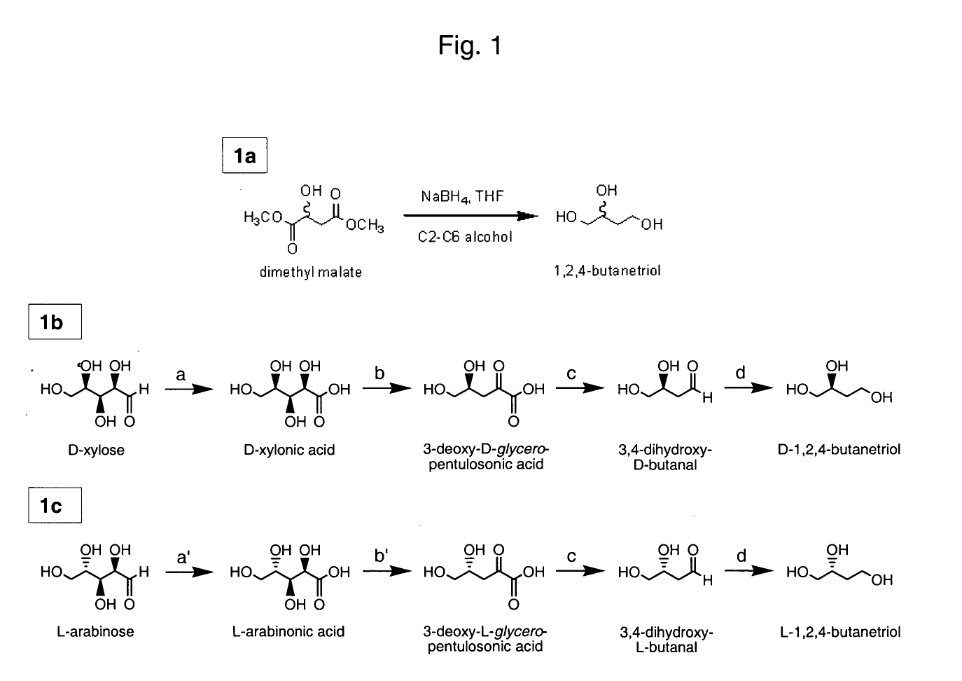

Discovery of novel D-xylose dehydrogenases. The first step of the D-1,2,4-butanetriol biosynthetic pathway utilizes a D-xylose dehydrogenase activity to covert D-xylose into D-xylonic acid (FIG. 1b). Although genes encoding this enzyme have been isolated from archaea and mammals, the expression of these reported enzymes in E. coli necessitated the use of special host strains to compensate for the differences in codon usages between different species. See, e.g., U. Johnsen & P. Schoenheit, Novel xylose dehydrogenase in the halophilic archaeon Haloarcula marisomortui, J. Bacteriol. 186:6198-6207 (2004); S. Aoki et al., Identification of dimeric dihydrodiol dehydrogenase as NADP⁺-dependent D-xylose dehydrogenase in pig liver, Chem. Biol. Inter. 130-132:775-784 (2001); and Y. Asada et al., Roles of His-79 and Tyr-180 of O— xylose dehydrogenase/dihydrodiol dehydrogenase in catalytic function, Biochem. Biophys. Res. Commun. 278:333-337 (2000). Thus, a D-xylose dehydrogenase that could be easily expressed in a regular E. coli strain is desirable for the construction of a D-1,2,4-butanetriol-synthesizing E. coli.

In a variety of xylose-metabolizing Pseudomonas strains, both the D-xylose dehydrogenase and the D-xylonic acid dehydratase have been reported as essential catabolic enzymes for D-xylose utilization. See, e.g., R. Weimberg, J. Biol. Chem. 236:629-635 (1961); and A. S. Dahms, 3-Deoxy-D-pentulosonic acid aldolase and its role in a new pathway of D-xylose degradation. Biochem. Biophys. Res. Commun. 60:1433-1439 (1974). We attempted to identify a D-xylose dehydrogenase-encoding gene by bioinformatic analysis of bacterial chromosomes. A BLAST analysis of the ERGO bacteria genome database using the partial amino acid sequence of D-xylonic acid dehydratase from P. fragi was performed.

A Burkholderia fungorum LB400 protein (see SEQ ID NO:2, encoded by SEQ ID NO:1), which was annotated by the ERGO bacteria genome database as the galactonate dehydratase, showed the highest homology score. In the previous analysis of the NCBI database, the same protein was also shown to contain amino acid sequences with high homology to all the five peptides resulting from the protease digestion of the purified D-xylonic acid dehydratase. When we examined the functions of ORFs adjacent to the proposed galactonate dehydratase, we identified one putative enzyme, designated as RBU11704 in the ERGO database, belonging to the short-chain dehydrogenase/reductase (SDR) superfamily. Because one major group of enzymes that constitutes the SDR superfamily is the carbohydrate dehydrogenases, exemplified by the glucose dehydrogenase, this B. fungorum protein was therefore considered as a D-xylose dehydrogenase candidate for further characterization. See, e.g., H. Joernvall et al., Short-chain dehydrogenases/reductases (SDR), Biochem. 34:6003-6013 (1995).

Examination of ORFs adjacent to other proteins with high homology to the partial D-xylonic acid dehydratase further revealed a second putative protein that belonged to the SDR superfamily. This Caulobacter crescentus CB 15 protein (see SEQ ID NO:4, encoded by SEQ ID NO:3), designated as RC001012 in the ERGO database, was encoded by a gene assigned as CC0821 in the CauloCyc (see the http internet site at biocyc.org) pathway/genome database of C. crescentus. The CC0821 gene has been previously proposed as one of two genes that could potentially encode a D-xylose dehydrogenase. See, e.g., A. K. Hottes et al., Transcriptional profiling of Caulobacter crescentus during growth on complex and minimal media, J. Bacteriol. 186:1448-1461 (2004). Protein sequence alignment showed that protein RC001012 has a 77% homology to protein RBU11704 from B. fungorum.

Characterization of the B. fungorum protein RBU11704 and the C. crescentus protein RC001012 utilized N-terminal 6×His-tagged fusion proteins purified by nickel/nitrilotriacetic acid (Ni-NTA) resin (available from QIAGEN Inc., Valencia, Calif., U.S.). Among the carbohydrates being tested, D-xylose, L-arabinose, and D-glucose could be oxidized into corresponding sugar acid under the catalysis of both enzymes. On the other hand, D-fructose, D-galactose, D-mannose, 2-deoxy-D-glucose, D-glucose-6-phosphate, and D-ribose were not the substrates for either enzyme.

In comparison to the two previously reported D-xylose dehydrogenases, which prefer NADP⁺ as the cofactor, the two bacteria enzymes showed more than 500-fold higher activities when NAD⁺ instead of NADP⁺ was provided as the cofactor. See, e.g., U. Johnsen & P. Schoenheit, J. Bacteriol. 186:6198-6207 (2004); and Y. Asada et al., Biochem. Biophys. Res. Commun. 278:333-337 (2000). Inclusion of divalent cations (Zn²⁺ or Fe²⁺) in the enzyme assays had no effect on the specific activities of the purified enzymes. The maximum activities of both enzymes were observed around pH 8.3. Analysis of enzyme kinetics revealed a significantly lower Km towards D-xylose relative to other carbohydrates for both dehydrogenases, while the Km(D-xylose) value of protein RC001012 (0.099 mM) was ten-fold lower than the Km(D-xylose) value of protein RBU11704 (0.97 mM) (Table 1). Furthermore, the C. crescentus enzyme is more active towards the C5 substrate L-arabinose but less active towards the C6 substrate D-glucose relative to the B. fungorum enzyme. As a D-xylose dehydrogenase, the C. crescentus enzyme is more efficient (kcat/Km) than the archaeal and the mammalian enzymes, while the B. fungorum enzyme has comparable catalytic efficiency to the reported enzymes (Table 1). We refer herein to the protein RBU11704 from B. fungorum LB400 and the protein RC001012 from C. crescentus CB15 in the ERGO database as D-xylose dehydrogenases (Xdh). Based on the kinetic data of the two enzymes, the D-xylose dehydrogenase from C. crescentus was selected to attempt to construct an of E. coli strain capable of synthesizing D-1,2,4-butanetriol from D-xylose.

TABLE 1
Kinetic data of D-xylose dehydrogenases

| | | | | | | L- | |
|---|---|---|---|---|---|---|---|
| Xylose | Cofactor | D-xylose | | D-glucose | | arabinose | |
| Dehydrogenase | Km | Km | kcat^{c} | Km | kcat | Km | kcat |
| and Source | (mM) | (mM) | (s⁻¹) | (mM) | (s⁻¹) | (mM) | (s⁻¹) |
| Xdh- | 0.26^{a} | 0.97 | 29 | 176 | 12 | 43 | 13 |
| B. fungorum | | | | | | | |
| Xdh- | 0.13^{a} | 0.099 | 41 | 538 | 24 | 34 | 40 |
| C. crescentus | | | | | | | |
| Xdh- | 0.15^{b} | 1.2 | 71 | — | — | — | — |
| H. marismortui⁸ | | | | | | | |
| mDD¹⁰ | 0.55^{b} | 6.4 | 4.8 | — | — | — | — |

^{a}Cofactor is NAD+.
^{b}Cofactor is NADP+.
^{c}Enzymes were considered as monomers in the calculations for all the kcat values.

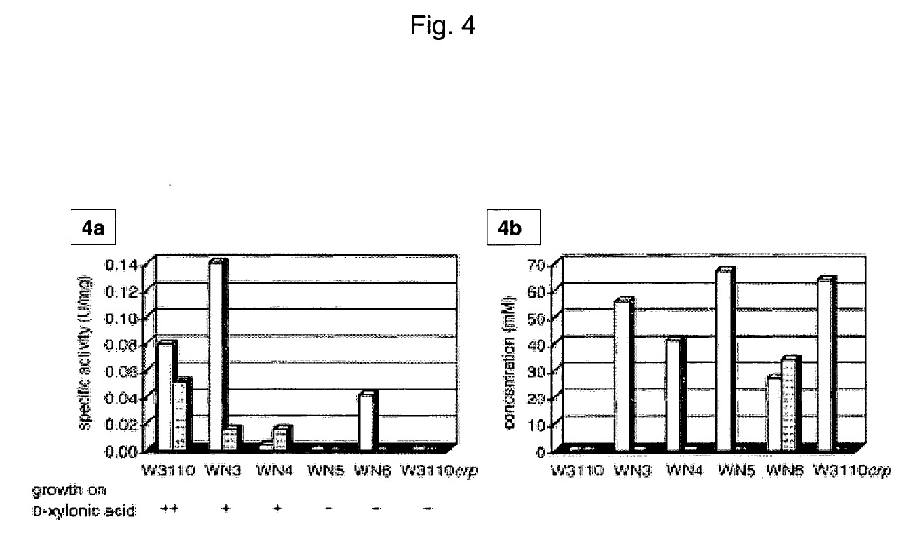

Elucidation of E. coli D-xylonic acid catabolic pathway. We have previously observed that E. coli K-12 wild-type strain W3110 could utilize D-xylonic acid as the sole source of carbon for growth via an unidentified catabolic pathway. See, e.g., W. Niu, Microbial synthesis of chemicals from renewable feedstocks. Ph.D. Thesis (Michigan State University, East Lansing, Mich., 2004). In the cell-free extract of thus cultivated W3110, we detected a D-xylonic acid dehydratase activity and a 3-deoxy-D-glycero-pentulosonic acid aldolase activity (FIG. 4a). Both activities were not detected in W3110 cells cultivated in media containing other common carbon sources such as D-glucose (see, e.g., W. Niu, ibid.). ¹H NMR analysis of catabolite accumulation further revealed that ethyleneglycol and glycolate were accumulated by W3110 cultured on D-xylonic acid. Both molecules were related to glycolaldehyde catabolism in E. coli. A D-xylose catabolic pathway has also been previously reported in Pseudomonas strains (see, e.g., R. Weimberg, J. Biol. Chem. 236:629-635 (1961); and A. S. Dahms, Biochem. Biophys. Res. Commun. 60: 1433-1439 (1974)).

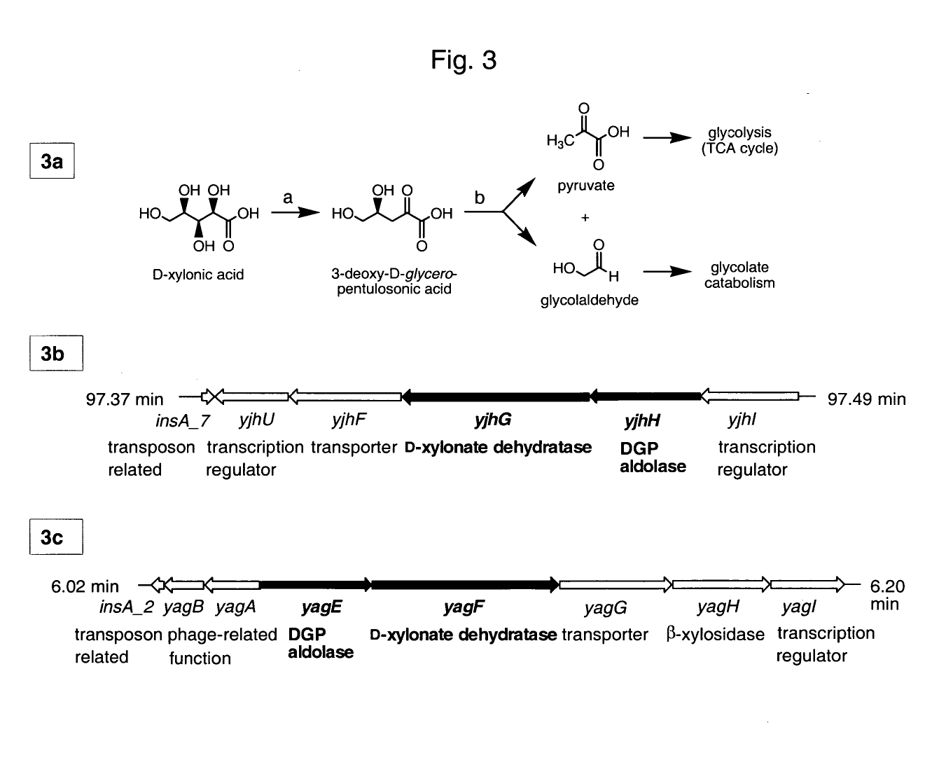

Using this information, we proposed a hypothetical pathway for E. coli catabolism of D-xylonic acid (FIG. 3a). In this pathway, D-xylonic acid is first converted into 3-deoxy-D-glycero-pentulosonic acid by the catalysis of a D-xylonic acid dehydratase, which also catalyzes the second step in D-1,2,4-butanetriol biosynthesis from D-xylose (FIG. 1b). The second step of the pathway involves an aldolase-catalyzed cleavage of the 2-keto acid intermediate to form pyruvate and glycolaldehyde. While the first reaction of the proposed pathway forms a key intermediate for D-1,2,4-butanetriol biosynthesis according to the present invention, the second reaction would divert this intermediate from biosynthesis to cell growth. Therefore, one strategy to improve E. coli biosynthesis of D-1,2,4-butanetriol is to use an E. coli strain which could not express functionally active 2-keto acid aldolase. As a result, all the 2-keto acid intermediate in the cells would be channeled to the biosynthetic pathway. However, successful application of this strategy would not be possible without validation of the proposed pathway and identification of genes encoding the proposed catabolic enzymes.

We first tried to elucidate the E. coli D-xylonic acid catabolic pathway by a random mutagenesis approach. Mutants of E. coli K-12 wild-type strain W3110 were generated using the EZ::Tn5™<R6Kyori/KAN-2> Tnp Transposome™ Kit (EPICENTRE Biotechnologies, Madison, Wis., U.S.). To isolate candidates that contained transposon insertion into genes crucial to the D-xylonic acid catabolism, the W3110 mutants were screened for the loss of ability to grow on M9 plates containing D-xylonic acid as the sole carbon source but retaining the same growth rate as the wild-type strain when cultured on M9 plates containing D-glucose as the sole carbon source. From 1,200 W3110 mutants, three candidates were identified using this phenotypic analysis. Two of the candidates had transposon inserted into the cya gene, which encodes the adenylate cyclase. See, e.g., M. Riley & B. Labedan, Escherichia coli gene products: physiological functions and common ancestries, In F. C. Neidhardt, (ed.), Escherichia coli and Salmonella: Cellular and Molecular Biology at 2118-2202 (2d ed.) (ASM Press,

Washington, D.C., 1996). The third candidate had transposon inserted into the crp gene, which encodes the cyclic AMP receptor protein (CRP). (F. C. Neidhardt, ibid.) As one of the global transcription regulators in E. coli, the binding of CRP to its DNA target is regulated by the cytoplasmic concentration of cAMP. See, e.g., M. H. Saier et al., Regulation of carbon utilization, In F. C. Neidhardt, (ed.), Escherichia coli and Salmonella: Cellular and Molecular Biology at 1325-1443 (2d ed.) (ASM Press, Washington, D.C., 1996). Studies also have shown that E. coli strains lacking adenylate cyclase activity have low cytoplasmic concentrations of cAMP. (M. H. Saier et al., ibid.)

Disruption of cya and/or crp genes resulted in catabolically repressed E. coli strains that could not grow on any carbon sources subject to catabolite repression. (M. H. Saier et al., ibid.) Therefore, we interpreted the isolation of the cya and the crp mutants which could not use D-xylonic acid as the sole carbon source for growth as an indication that E. coli catabolism of D-xylonic acid is regulated by catabolite repression. To avoid repetitive isolation of mutants with impaired regulation on catabolite repression, we used a third type of M9 plate that contained glycerol as the sole carbon source to screen an additional 2,500 W3110 mutants. Because the catabolism of glycerol by E. coli is also regulated by catabolite repression, we instead looked for W3110 mutants that could grow on both D-glucose and glycerol as the sole carbon source but could not grow on D-xylonic acid as the sole carbon source. However, surprisingly no mutant with such a phenotype was observed. The random mutagenesis experiment was not able to reveal any structural genes associated with an E. coli D-xylonic acid catabolic pathway.

In a further attempt to understand E. coli catabolism of D-xylonic acid, a bioinformatic analysis of the E. coli K-12 genome was performed, starting with a BLAST search using the partial amino acid sequence of the P. fragi D-xylonic acid dehydratase. We identified four candidate dehydratases with a sequence identity to the query sequence ranging from 32-41%. In addition to two well-studied enzymes, 6-phosphogluconate dehydratase and dihydroxyacid dehydratase, the other two uncharacterized putative dehydratases were encoded by gene yjhG (97.424 min) and gene yagF (6.0872 min). Examination of the E. coli genome regions upstream and downstream of yjhG and yagF revealed two sets of genes that encoded putative DNA transcription repressor proteins (yjhI and yagI), putative transporter proteins (yjhF and yagG), and putative aldolases/synthases (yjhH and yagE) (FIG. 3b). An additional gene (yagH) that encoded a putative P-xylosidase also located near the yagF gene. The structures of both sets of genes resembled the structures of other E. coli catabolic pathway encoding genes exemplified by the lac operon. Another intriguing observation was that both sets of genes encoded enzymes that are essential and sufficient for a regulated D-xylonic acid catabolism via our proposed pathway (FIG. 3a). For future convenience, we named the two sets of genes as yjh gene cluster and yag gene cluster.

To investigate the possible roles of the yjh and the yag gene clusters in E. coli catabolism of D-xylonic acid, we first tested the in vitro activities of the two putative dehydratases and the two putative aldolases/synthases. PCR-amplified DNA products of gene yjhG, yagF, yjhH, and yagE were respectively cloned into protein expression vector pJF118EH. The cell-free lysate of E. coli cells expressing the target enzymes was used in the analysis. Using ¹H NMR, we were able to detect the formation of 3-deoxy-glycero-pentulosonic acid from D-xylonic acid in enzymatic reactions catalyzed by the lysates of E. coli expressing YjhG or YagF. ¹H NMR analysis also showed that the two putative aldolases/synthases encoded by yjhH and yagE could catalyze the conversion from 3-deoxy-D-glycero-pentulosonic acid into pyruvate and glycolaldehyde. We further verified the aldolase activities of YjhH and YagE using a spectrophotometric method. By inclusion of the lactate dehydrogenase in the enzymatic reactions, the aldolase-catalyzed formation of pyruvate from 3-deoxy-D-glycero-pentulosonic acid was monitored by the oxidation of NADH. These results suggested that YjhG and YagF indeed had D-xylonic acid dehydratase activities; moreover, YjhH and YagE indeed had 3-deoxy-D-glycero-pentulosonic acid aldolase activities.

Next, we examined whether the yjh and the yag gene clusters were essential for E. coli catabolism of D-xylonic acid. Because the goal of elucidating E. coli D-xylonic acid catabolic pathway was to explore the possibility of constructing an E. coli mutant that could not consume 3-deoxy-D-glycero-pentulosonic acid and to evaluate the effect of such a catabolic modification on E. coli biosynthesis of D-1,2,4-butanetriol, genes encoding the two aldolases (yjhH and yagE) were targeted for chromosomal knockout experiments. Four E. coli mutants were generated from wild-type strain W3110. E. coli WN3 and WN4 were two single knockout strains. Replacement of a partial DNA sequence of the yjhH gene on the chromosome of W3110 with a gene encoding a chloramphenicol-resistance protein resulted in strain WN3 (Table 2). Replacement of a partial DNA sequence of the yagE gene on the chromosome of W3110 with a gene encoding a kanamycin-resistance protein resulted in strain WN4 (Table 2). E. coli WN5 was a double knockout strain which contained both mutations from strain WN3 and WN4 (Table 2).

TABLE 2
Bacterial strains and plasmids

| Strain/Plasmid | Relevant Characteristics | Reference/Source |
|---|---|---|
| Burkholderia fungorum | wild-type | ARS |
| LB400 | | |
| Caulobacter crescentus | wild-type | ATCC |
| CB15 | | |
| Pseudomonas fragi | wild-type | ATCC |
| DH5α | lacZΔM15 hsdR recA | Invitrogen |
| W3110 | wild-type K-12 | CGSC |
| W3110cya | W3110cya::Kan^{R} | this study |
| W3110crp | W3110crp::Kan^{R} | this study |
| WN3 | W3110yjhH::Cm^{R} | this study |
| WN4 | W3110yagE::Kan^{R} | this study |
| WN5 | W3110yjhH::Cm^{R}yagE::Kan^{R} | this study |
| WN6 | W3110ΔyjhHΔyagE | this study |
| WN7 | W3110ΔyjhHΔyagEserA | this study |
| W3110serA | W3110serA | this study |
| WNI3 | WN7xylAB::xdh-Cm^{R} | this study |
| pKD3 | Ap^{R}, Cm^{R} | ref 27 |
| pKD4 | Ap^{R}, Kan^{R} | ref 27 |
| pKD46 | Kan^{R} | ref 27 |
| pCRTOP02.1 | Kan^{R} | Invitrogen |
| pQE30 | Ap^{R} | Qiagen |
| pJG7.246 | Ap^{R}, lacl^{Q} in pQE30 | lab strain |
| pJF118EH | Ap^{R}, P_{tac} lacl^{O} | ref 26 |
| pRC1.55B | Cm^{R}, serA in pSU18 | lab strain |
| pWN7.270A | Ap^{R}, yjhG in pJF118EH | this study |
| pWN7.272A | Ap^{R}, yagF in pJFI18EH | this study |
| pWN8.020A | Ap^{R}, yagE in pJF118EH | this study |
| pWN8.022A | Ap^{R}, yjhH in pJF118EH | this study |
| pWN9.044A | Ap^{R}, xdh (B. fungorum) in | this study |
| | pJG7.246 | |
| pWN9.046A | Ap^{R}, xdh (C. crescentus) in | this study |
| | pJG7.246 | |
| pWN7.126B | Ap^{R}, serA in pWN5.238° | this study |
| pWN9.068A | Ap^{R}, xdh (C. crescentus) in pKD3 | this study |
| KIT4 | WN7xylAB::xdh-adhP-Ptac-FRT | this study |
| KIT10 | WN7xylAB::xdh-FRT adhP::FRT | this study |
| KIT18 | WN7xylAB::xdh-adhP-Ptac- | this study |
| | FRTyiaE::FRTycdW::FRT | |

Computer analysis has shown that each dehydratase-encoding gene shares a potential promoter sequence with the upstream aldolase-encoding gene (FIG. 3b). To alleviate the potential polar mutation effect on the expressions of dehydratases caused by gene insertion into the aldolase-encoding genes, a fourth E. coli mutant WN6 was generated by removal of the two antibiotic resistant gene markers from the chromosome of strain WN5 (Table 2). The four mutant strains were then evaluated for growth characters on M9 solid mediums (FIG. 4a). E. coli wild-type strain W3110 and the catabolically repressed strain W3110crp were included as controls in these experiments. When glucose was provided as the sole carbon source, all the four mutant strains had similar growth rates as strain W3110 on M9 plates. However, when D-xylonic acid was provided as the sole carbon source, only the two single knockout mutant strains were able to grow on M9 plates, but with a slower rate relative to the wild-type control strain (FIG. 4a). Unambiguous growth of E. coli WN5, WN6, and W3110crp was not detected on the same medium after 72 h of incubation at 37° C. (FIG. 4a). These observations indicated that the slower growth rates of WN3 and WN4 on D-xylonic acid were caused by lower activities of catabolic proteins directly related to the D-xylonic acid utilization. And because of the complete absence of these catalytic activities, E. coli WN5 and WN6 lost the ability to utilize D-xylonic acid as a sole carbon source for growth.

We further analyzed the four mutant strains for the expression of the two D-xylonic acid catabolic enzymes, D-xylonic acid dehydratase and 3-deoxy-D-glycero-pentulosonic acid aldolase. The enzyme assays utilized the cell-free lysate of individual strain that was cultivated in LB medium containing D-xylonic acid. The two single knockout E. coli mutants, WN3 and WN4, expressed both the dehydratase and the aldolase (FIG. 4a). Due to the predicted polar mutation effects, the double knockout mutant WN5 did not express either of the catabolic enzymes (FIG. 4a). On the other hand, the marker-free mutant strain WN6 recaptured the ability to express the D-xylonic acid dehydratase, while was still depleted with the 3-deoxy-D-glycero-pentulosinc acid aldolase activity (FIG. 4a). Using ¹H NMR, we also monitored the D-xylonic acid consumption and the catabolite accumulation of the cell cultures subjected to enzyme expression analysis. At the end of the cultivation, E. coli WN5 and W3110crp didn't consume any D-xylonic acid. The wild-type E. coli strain W3110 consumed all the D-xylonic acid in the medium, while the two single knockout strains and WN6 only consumed part of the acid (FIG. 4b). Among the six strains, E. coli WN6 was the only strain that secreted the substrate of the aldolase, 3-deoxy-D-glycero-pentulosonic acid, into the medium (FIG. 4b).

Up to this point, the results obtained from both the in vitro and the in vivo experiments verified that E. coli catabolism of D-xylonic acid followed our proposed pathway (FIG. 3a). Furthermore, two copies of the required catabolic enzymes were encoded by genes belonging to the yjh and the yag gene clusters.

Microbial synthesis of D-1,2,4-butanetriol. We first evaluated the effect of eliminating the 3-deoxy-D-glycero-pentulosonic acid aldolase activity on E. coli synthesis of D-1,2,4-butanetriol from D-xylonic acid. Two E. coli host strains, W3110 serA and WN7, were constructed for this purpose. W3110serA was directly derived from wild-type strain W3110 (Table 2) and WN7 was directly derived from strain WN6 (Table 2). The two host strains shared the same mutated serA gene located on the chromosome. The serA gene encodes 3-phosphoglycerate dehydrogenase, which is necessary for the biosynthesis of L-serine. Therefore, E. coli strain lacking this enzymatic activity could only grow in minimal salts medium without L-serine supplementation when the cells successfully maintained a SerA-encoding plasmid. This nutrient pressure strategy has been used extensively as an effective means of plasmid maintenance. See, e.g., K. M. Draths et al., Shikimic acid and quinic acid: replacing isolation from plant sources with recombinant microbial biocatalysis, J. Am. Chem. Soc. 121:1603-1604 (1999). In addition to the serA gene, plasmid pWN7.126B also contained an mdlC gene isolated from P. putida (ATCC 12633) (Table 2) (see SEQ ID NO:44, encoded by SEQ ID NO:43). The md/C gene encodes the 2-keto acid decarboxylase, which is the enzyme that catalyzes the third step in the D-1,2,4-butanetriol biosynthetic pathway (FIG. 1b).

The microbial syntheses were carried out in minimal salts mediums under fermentor controlled cultivation conditions at 33° C., pH 7.0, with dissolved oxygen level maintained at 10% air saturation. See, e.g., K. Li et al., Fed-batch fermentor synthesis of 3-dehydroshikimic acid using recombinant Escherichia coli, Biotechnol. Bioeng. 64:61-73 (1999). Glucose was provided as the sole carbon source for cell growth. A solution containing potassium D-xylonate was added into the culture medium as the biosynthetic starting material. To avoid catabolite repression on the expression of D-xylonic acid catabolic enzymes caused by high glucose concentration in the culture medium, the steady state glucose concentrations were maintained at approximately 0.2 mM. After 48 h of cultivation, E. coli W3110serA/pWN7.126B, which had functional D-xylonic acid catabolic pathways, only synthesized 0.08 g/L of D-1,2,4-butanetriol from 18 g of D-xylonic acid in 0.75% yield (FIG. 5a).

In contrast, E. coli WN7/pWN7.126B, which could express catalytically active D-xylonic acid dehydratases but not 3-deoxy-D-glycero-pentulosonic acid aldolases, synthesized 8.3 g/L of D-1,2,4-butanetriol from 28 g of D-xylonic acid in 45% yield (FIG. 5a). The results therefore demonstrated that inactivation of the 3-deoxy-D-glycero-pentulosonic acid aldolase was a successful strategy to improve E. coli synthesis of D-1,2,4-butanetriol from D-xylonic acid in minimal salts medium.

However, disruption of D-xylonic acid catabolic pathways in E. coli biocatalyst should in theory lead to a 100% conversion from D-xylonic acid to D-1,2,4-butanetriol. To understand the flow of carbons derived from D-xylonic acid during the biosynthesis, we analyzed the fermentation broth of strain WN7/pWN7.126B for byproduct formation. After removal of the cells, broth harvested after 48 h of cultivation was purified using Dowex 1 (CI⁻ form) and Dowex 50 (H⁺ form) ion exchange resins. The solute contents at each purification step were analyzed using ¹H NMR. We thus detected 3-deoxy-D-glycero-pentulosonic acid, 3-deoxy-D-glycero-pentanoic acid, (4S) 2-amino-4,5-dihydroxy pentanoic acid, and D-3,4-dihydroxy butanoic acid (FIG. 5d). The first molecule is a designated biosynthetic intermediate. The second and the third molecule could respectively be a reduction and a transamination product of this intermediate. Accumulation of these three byproducts indicated a mismatch between the in vivo catalytic activity of the D-xylonic acid dehydratase, which catalyzed the formation of 3-deoxy-D-glycero-pentulosonic acid, and the in vivo catalytic activity of the 2-keto acid decarboxylase, which catalyzed the conversion of this 2-keto acid into D-3,4-dihydroxybutanal (FIG. 1b). To understand the mechanism of D-3,4-dihydroxy butanoic acid formation, we also analyzed the fermentation broth of E. coli WN7/pRC1.55B, which didn't express the 2-keto acid decarboxylase. However, this organic acid was not detected in the purified broth. Therefore, D-3,4-dihydroxy butanoic acid is very likely to be an oxidation product of 3,4-dihydroxybutanal (FIG. 5d)

We proceeded to examine E. coli synthesis of D-1,2,4-butanetriol directly from D-xylose in minimal salts medium by the construction of host strain WN13. E. coli WN13 was derived from strain WN7 by replacing the genomic copy of xylAxylB gene cluster with a xdh(C. crescentus)-Cm^{R} gene cassette (Table 2). The xylA gene encodes the D-xylose isomerase. The xylB gene encodes the D-xylulose kinase. These are two enzymes essential for E. coli catabolism of D-xylose. The chromosomal modification of WN13 therefore abolished its ability to utilize D-xylose as a sole carbon source for growth. As a second consequence, E. coli WN 13 could express a D-xylose dehydrogenase activity under the control of the xylA promoter. Biosynthesis of D-1,2,4-butanetriol by E. coli WN13/pWN7.126B was evaluated under the similar fermentor controlled cultivation conditions as described above. The only change was that D-xylose instead of D-xylonic acid was added into the culture medium as the biosynthetic starting material at indicated time points (FIG. 5b). After 48 h of cultivation, E. coli WN13/pWN7.126B synthesized 6.2 g/L of D-1,2,4-butantriol from 30 g of D-xylose in 30% yield (FIG. 5a). The same biosynthetic byproducts accumulated by strain WN7/pWN7.126B were also detected in the culture medium of strain WN13/pWN7.126B. Analysis of the D-xylose dehydrogenase specific activities throughout the cultivation process showed that the expression of this enzyme was induced by D-xylose (FIG. 5c). This result indicated that the chromosomal integration of xdh gene was successful.

As a result of these discoveries and recombinant strain construction, improved biocatalysis of 1,2,4-butanetriol is now possible as a commercial option that offers stereo-selectivity, the use of mild reaction conditions, and the environmental benign nature of the process. The microbial synthesis of D-1,2,4-butanetriol followed such an artificial biosynthetic pathway (FIG. 1b) which was built around an oxidative D-xylose catabolic pathway utilized by certain gram-negative bacteria. See, e.g., R. Weimberg, J. Biol. Chem. 236:629-635 (1961); and A. S. Dahms, Biochem. Biophys. Res. Commun. 60: 1433-1439 (1974). Various embodiments of the present invention improve this pathway and its level of 1,2,4-butanetriol production, including embodiments in which a single host cell can perform a xylose-to-1,2,4-butranetriol synthesis, and in various embodiments can do so on minimal salts medium.

The elucidation of a previously unreported E. coli D-xylonic acid catabolic pathway (FIG. 3a) has now permitted the realization of D-1,2,4-butanetriol biosynthesis in minimal salts medium. Two sets of catabolic enzymes encoded by the yjh and the yag gene clusters were discovered in E. coli K-12 wild-type strain (FIG. 3b). Chromosomal knockout experiments showed that enzymes encoded by either gene cluster are sufficient for E. coli utilization of D-xylonic acid as the sole carbon source for growth (FIG. 4a). Furthermore, the polar mutation effect observed in mutant strain WN5 (FIG. 4) indicated that the genes encoding the 3-deoxy-D-glycero-pentulosonic acid aldolases (yjhH and yagE) and the genes encoding the D-xylonic acid dehydratases (yjhG and yagF) formed two transcription operons. The expression of catabolic enzymes encoded by both gene clusters are induced by D-xylonic acid and also tightly regulated under catabolite repression. The presence of two copies of genes encoding the same enzymatic activities explained why the transposon random mutagenesis experiment, which could only efficiently mutate one gene at a time, was unable to reveal structural genes for the D-xylonic acid catabolic pathway.

The identification of genes encoding the D-xylonic acid dehydratase and the 3-deoxy-D-glycero-pentulosonic acid aldolase will also facilitate future kinetic and structural studies of the two enzymes. Our preliminary enzyme assays showed that the two aldolases encoded by gene yjhH and gene yagE could catalyze the cleavage of both the D- and the L-3-deoxy-glycero-pentulosonic acid isomers (data not shown). These two enzymes therefore join a 2-keto-3-deoxygluconate aldolase isolated from Sulfolobus solfataricus as member of the few aldolases that catalyze non-stereo-specific aldo reactions. See, e.g., A. Theodossis et al., The structural basis for substrate promiscuity in 2-keto-3-deoxygluconate aldolase from the Entner-Doudoroff pathway in Sulfolobus solfataricus, J. Biol. Chem. 279:43886-43892 (2004). Likewise, these aldolases encoded by gene yjhH and gene yagE can be usefully inactivated or inhibited to enhance production of L-1,2,4-butanetriol in biosynthetic pathways using an L-arabinose or L-arabinonate source as a starting material.

The E. coli synthesis of D-1,2,4-butanetriol directly from D-xylose also benefits from the discovery of novel bacterial D-xylose dehydrogenases (Xdh). In addition to having catalytic efficiencies comparable to those of previously reported enzymes (Table 1), the novel D-xylose dehydrogenases from B. fungorum and C. crescentus can be efficiently expressed as catalytically active forms in commonly used E. coli production strains. Thus, these two D-xylose dehydrogenases can be utilized in a variety of common bacterial production strains for 1,2,4-butranetriol or other desired products.

To reduce the cost associated with biocatalyst preparation, the D-1,2,4-butanetriol synthesizing E. coli has now been constructed from a host strain that lost the ability to grow on D-xylose and D-xylonic acid as the sole carbon source. As a consequence, E. coli WN13/pWN7.126B was cultivated on D-glucose, which is a cheaper starting material relative to D-xylose. The biocatalyst utilized D-xylose solely for the biosynthetic purpose. In addition to producing the biosynthetic target, D-1,2,4-butanetriol, and the designed biosynthetic intermediate, 3-deoxy-D-glycero-pentulosonic acid, E. coli WN13/pWN7.126B was also found to synthesize other useful molecules that were not previously reported as common bacterial metabolites', including 3-deoxy-D-glycero-pentanoic acid, (4S) 2-amino-4,5-dihydroxy pentanoic acid, and D-3,4-dihydroxy butanoic acid (FIG. 5d). In various embodiments hereof, one or more of the enzymes can be inhibited or inactivated to decrease or eliminate the formation of these byproducts and thereby improve biosynthesis of D-1,2,4-butanetriol further.

Nevertheless, the multiple stereocenters in the byproducts can be exploited as valuable chiral synthons for chemical syntheses. Genetic modification of the E. coli WN13/pWN7.1268 could potentially lead to new strains to synthesize the “byproduct” as the target molecule. The expanded molecular diversity of the D-1,2,4-butanetriol biosynthetic pathway revealed the flexibility of a bacterial catalytic network, which is an observation echoes the “enzyme recruitment” theory for natural biosynthetic pathway evolution. See, e.g., R. A. Jensen, Enzyme recruitment in evolution of new function, Ann. Rev. Microbiol. 30: 409-425 (1976); and S. Schmidt et al., Metabolites: a helping hand for pathway evolution? Trends. Biochem. Sci. 28:336-341 (2003). Integration of foreign catalytic activities including D-xylose dehydrogenase and 2-keto acid decarboxylase into E. coli native catalytic network resulted in the rewiring of the carbon flow and the biosynthesis of novel metabolites.

### Materials and Methods

Chemicals and culture media. Potassium xylonate used for fermentation was prepared as previously described. See, W. Niu et al., J. Am. Chem. Soc. 125:12998-12999 (2003). Chemically synthesized potassium xylonate was used for enzyme assay and medium preparation. See, e.g., S. Morre & K. P. Link, Carbohydrate characterization: I. The oxidation of aldoses by hypoiodite in methanol; and II. The identification of seven aldo-monosaccharides as benzimidazole derivatives, J. Biol. Chem. 133:293-311 (1940). The 3-deoxy-D,L-glycero-pentulosonic acid was chemically synthesized. See, e.g., A. C. Stoolmiller, DL- and L-2-Keto-3-deoxyarabonate-1,2. Methods in Enzymol. 41:101-103 (1975). All the other chemicals were purchased from commercial resources.

All solutions were prepared in distilled, deionized water. LB medium (see, e.g., J. H. Miller, Experiments in Molecular Genetics (Cold Spring Harbor Laboratory Press, Plainview, N.Y., 1972)) (1 L) contained Bacto tryptone (10 g), Bacto yeast extract (5 g), and NaCl (10 g). M9 salts (1 L) contained Na₂HPO₄(6 g), KH₂PO₄(3 g), NH₄Cl (1 g), and NaCl (0.5 g). M9 minimal medium contained D-glucose (10 g), MgSO₄(0.12 g), and thiamine hydrochloride (0.001 g) in 1 L of M9 salts. M9 D-xylonic acid medium contained potassium D-xylonate (10 g) in place of D-glucose in M9 minimal salts. M9 glycerol medium contained glycerol (10 g) in place of D-glucose, in M9 minimal salts. Antibiotics were added where appropriate to the following final concentrations: ampicillin (Ap), 50 μg/mL; chloramphenicol (Cm), 20 μg/mL, and kanamycin (Kan), 50 μg/mL. Isopropyl-(3-D-thiogalactopyranoside (IPTG) was prepared as a 500 mM stock solution. Solutions of M9 salts, MgSO₄, glucose, and glycerol were autoclaved individually and then mixed. Solutions of potassium D-xylonate, thiamine hydrochloride, antibiotics, and IPTG were sterilized through 0.22-μm membranes. Solid mediums were prepared by addition of Difco agar to a final concentration of 1.5% (w/v) to the liquid medium.

The standard fermentation medium (1 L) contained K₂HPO₄(7.5 g), ammonium iron (III) citrate (0.3 g), citric acid monohydrate (2.1 g), and concentrated H₂SO₄(1.2 mL). Fermentation medium was adjusted to pH 7.0 by addition of concentrated NH₄OH before autoclaving. The following supplements were added immediately prior to initiation of the fermentation: D-glucose, MgSO₄ (0.24 g), and trace minerals including (NH₄)₆(Mo₇O₂₄).4H₂O (0.0037 g), ZnSO₄.7H₂O (0.0029 g), H₃BO₃(0.0247 g), CuSO₄.5H₂O (0.0025 g), and MnCl₂.4H₂O (0.0158 g). IPTG stock solution was added as necessary to the indicated final concentration. Glucose and MgSO₄(1 M) solutions were autoclaved separately. Antifoam 204 (Sigma-Aldrich Corp., St. Louis, Mo., U.S.) was added as needed.

Nucleotide and Amino Acid Sequences. Nucleotide and amino acid sequences are identified in Table 3.

TABLE 3
Identities of Listed Sequences

| SEQ ID NO | IDENTITY |
|---|---|
| SEQ ID NO: 1 | DNA coding sequence for Burkholderia fungorum LB400 xylose dehydrogenase |
| | (gene xdh; RBU11704) |
| SEQ ID NO: 2 | Amino acid sequence of Burkholderia fungorum LB400 xylose dehydrogenase |
| | (Xdh) |
| SEQ ID NO: 3 | DNA coding sequence for Caulobacter crescentus CB15 xylose dehydrogenase |
| | (gene xdh; RCO01012) |
| SEQ ID NO: 4 | Amino acid sequence of Caulobacter crescentus CB15 xylose dehydrogenase |
| | (Xdh) |
| SEQ ID NO: 5 | DNA coding sequence for E. coli xylonate dehydratase (gene yjhG) |
| SEQ ID NO: 6 | Amino acid sequence of E. coli xylonate dehydratase (YjhG) |
| SEQ ID NO: 7 | DNA coding sequence for E. coli xylonate dehydratase (gene yagF) |
| SEQ ID NO: 8 | Amino acid sequence of E. coli xylonate dehydratase (YagF) |
| SEQ ID NO: 9 | DNA coding sequence for Pseudomonas fragi (ATCC 4973) xylonate |
| | dehydratase fragment |
| SEQ ID NO: 10 | Amino acid sequence of Pseudomonas fragi (ATCC 4973) xylonate |
| | dehydratase fragment. |
| SEQ ID NO: 11 | DNA coding sequence for E. coli 3-deoxy-D-glycero-pentulosonate aldolase |
| | (gene yjhH) |
| SEQ ID NO: 12 | Amino acid sequence of E. coli 3-deoxy-D-glycero-pentulosonate aldolase |
| | (YjhH) |
| SEQ ID NO: 13 | DNA coding sequence for E. coli 3-deoxy-D-glycero-pentulosonate aldolase |
| | (gene yagE) |
| SEQ ID NO: 14 | Amino acid sequence of E. coli 3-deoxy-D-glycero-pentulosonate aldolase |
| | (YagE) |
| SEQ ID NO: 15 | Forward primer for Berkholderia fungorum LB400 xdh gene |
| SEQ ID NO: 16 | Reverse primer for Berkholderia fungorum LB400 xdh gene |
| SEQ ID NO: 17 | Forward primer for Caulobacter crescentus CB15 xdh gene |
| SEQ ID NO: 18 | Reverse primer for Caulobacter crescentus CB15 xhd dgene |
| SEQ ID NO: 19 | Forward primer for E. coli W3110 D-xylonate dehydratase gene (yjhG) |
| SEQ ID NO: 20 | Reverse primer for E. coli W3110 D-xylonate dehydratase gene (yjhG) |
| SEQ ID NO: 21 | Forward primer for E. coli W3110 D-xylonate dehydratase gene (yagF) |
| SEQ ID NO: 22 | Reverse primer for E. coli W3110 D-xylonate dehydratase gene (yagF) |
| SEQ ID NO: 23 | Forward primer for E. coli W3110 3-deoxy-D-glycero-pentulosonate aldolase |
| | gene (yjhH) |
| SEQ ID NO: 24 | Reverse primer for E. coli W3110 3-deoxy-D-glycero-pentulosonate aldolase |
| | gene (yjhH) |
| SEQ ID NO: 25 | Forward primer for E. coli W3110 3-deoxy-D-glycero-pentulosonate aldolase |
| | gene (yagE) |
| SEQ ID NO: 26 | Reverse primer for E. coli W3110 3-deoxy-D-glycero-pentulosonate aldolase |
| | gene (yagE) |
| SEQ ID NO: 27 | Forward primer for C. crescentus CB15 D-xylose dehydrogenase gene, for |
| | construction of plasmid pWN9.068A |
| SEQ ID NO: 28 | Reverse primer for C. crescentus CB15 D-xylose dehydrogenase gene, for |
| | construction of plasmid pWN9.068A |
| SEQ ID NO: 29 | Forward primer for Pseudomonas fragi xylonate dehydratase gene. |
| SEQ ID NO: 30 | Reverse primer for Pseudomonas fragi xylonate dehydratase gene. |
| SEQ ID NO: 31 | Forward primer for the DNA fragment used to disrupt E. coli genomic 3-deoxy- |
| | D-glycero-pentulosonate aldolase gene (yjhH) |
| SEQ ID NO: 32 | Reverse primer for the DNA fragment used to disrupt E. coli genomic 3-deoxy- |
| | D-glycero-pentulosonate aldolase gene (yjhH) |
| SEQ ID NO: 33 | Forward primer for the DNA fragment used to disrupt E. coli genomic 3-deoxy- |
| | D-glycero-pentulosonate aldolase gene (yagE) |
| SEQ ID NO: 34 | Reverse primer for the DNA fragment used to disrupt E. coli genomic 3-deoxy- |
| | D-glycero-pentulosonate aldolase gene (yagE) |
| SEQ ID NO: 35 | Forward primer for the DNA fragment used to insert xdh into E. coli genomic DNA |
| SEQ ID NO: 36 | Reverse primer for the DNA fragment used to insert xdh into E. coli genomic DNA |
| SEQ ID NO: 37 | DNA coding sequence for E. coli alcohol dehydrogenase (gene adhP) |
| SEQ ID NO: 38 | Amino acid sequence of E. coli alcohol dehydrogenase (AdhP) |
| SEQ ID NO: 39 | DNA coding sequence for E. coli 2-keto acid dehydrogenase (gene yiaE) |
| SEQ ID NO: 40 | Amino acid sequence of E. coli 2-keto acid dehydrogenase (YiaE) |
| SEQ ID NO: 41 | DNA coding sequence for E. coli 2-keto acid dehydrogenase (gene ycdW) |
| SEQ ID NO: 42 | Amino acid sequence of E. coli 2-keto acid dehydrogenase (YcdW) |
| SEQ ID NO: 43 | DNA coding sequence for Pseudomonas putida 2-keto acid decarboxylase |
| | (gene mclC) |
| SEQ ID NO: 44 | Amino acid sequence of Pseudomonas putida 2-keto acid decarboxylase |
| | (MdlC) |

Note that in SEQ ID NO: 11, nt1-3 show the putative initiator codon, whereas nt55-57 show an alternative initiator codon that makes nt55-960 the coding sequence for the alternative YjhH 3-deoxy-D-glycero-pentulosonate aldolase polypeptide. Similarly, in SEQ ID NO: 12, Met(1) is the putative initiator Met, and Met(19) is the alternative initiator Met, with Met(19)-Val(319) being the alternative YjhH 3-deoxy-D-glycero-pentulosonate aldolase polypeptide.

Bacterial strains and plasmids. E. coli K-12 strain W3110 was obtained from the E. coli Genetic Stock Center (Yale University, New Haven, Conn., U.S.). Plasmid constructions were carried out in E. coli DH5α, which was obtained from Life Technologies Inc. (Rockville, Md., U.S.). Pseudomonas fragi (ATCC 4973) and Caulobacter crescentus (ATCC 19089) were obtained from the American Type Culture Collection (Manassas, Va., U.S.). Burkhoideria fungorum LB400 was obtained as Accession No. NRRL B-18064 from ARS Patent Culture Collection (United States Department of Agriculture, Peoria, Ill., U.S.). Plasmid pJFI18EH (see, e.g., J. P. Furste et al., Molecular cloning of the plasmid Rp4 primase region in a multi-host-range tacP expression vector, Gene 48:119-131 (1986)) was generously provided by Professor M. Bagdasarian of Michigan State University. Homologous recombinations utilized plasmid pKD3, pKD4, pKD46, and pCP20 (see, K. A. Datsenko & B. L. Wanner, One step inactivation of chromosomal genes in Escherichia coli K-12 using PCR products, Proc. Natl. Acad. Sci USA 97:6640-6645 (2000)), which were obtained from the E. coli Genetic Stock Center. Plasmid pCRTOP02.1 was purchased from Invitrogen Corp. (Carlsbad, Calif., U.S.). Plasmid pQE30 was purchased from QIAGEN, Inc. All strains and plasmids used herein are summarized in Table 2.

General molecular biology and plasmid construction. Standard protocols were used for construction, purification, and analysis of plasmid DNA. J. Sambrook & D. W. Russell, Molecular Cloning, a Laboratory Manual (3d ed., 2001) (Cold Spring Harbor Lab. Press, Cold Spring Harbor, N.Y.). E. coli genomic DNA was isolated according to the procedure described in D. G. Pitcher et al., “Rapid extraction of bacterial genomic DNA with guanidium thiocyanate,” Lett. Appl. Microbiol. 8:151-56 (1989). Genomic DNA isolations from other bacterial strains followed a previously established method of K. Wilson, “Preparation of genomic DNA from bacteria,” in Current Protocols in Molecular Biology (F. M. Ausubel et al., eds.) 2.4.1-2.4.5 (1987) (Wiley, NY). Fast-Link™ DNA ligation kit was purchased from EPICENTRE Biotechnologies. DNA polymerase I (Klenow fragment) and calf intestinal alkaline phosphatase were purchased from Invitrogen Corp. PCR amplifications were carried out as described in Sambrook & Russell (2001). PfuTurbo® DNA polymerase was purchased from Stratagene Corp. (LaJolla, Calif., U.S.). Primers were synthesized by the Macromolecular Structure Facility at Michigan State University (East Lansing, Mich., U.S.). DNA sequencing service was provided by the Genomic Technology Support Facility at Michigan State University.

The xdh gene from B. fungorum LB400 was amplified from the genomic DNA isolated from the desired strain using the following forward and reverse primers with BamHI restriction sites underlined: 5′-CGGGATCCATGTATTTGTTGTCATACCC (SEQ ID NO:15) and 5′-CGGGATCCATATCGACGAAATAAACCG (SEQ ID NO:16). Digestion of the resulting DNA with BamHI followed by ligation into the BamHI site of pJG7.246 resulted in plasmid pWN9.044A. Plasmid pWN9.046A contained the gene encoding C. crescentus CB15 D-xylose dehydrogenase. This plasmid was constructed using the same strategy as for pWN9.044A. The following primers were used to amplify the xdh gene from the genomic DNA of C. crescentus CB15, 5′-GCGGATCCATGTCCTCAGCCATCTATCC (SEQ ID NO:17) and 5′-GCGGATCCGATGACAGTTTTCTTAGGTC (SEQ ID NO:18).

E. coli genes were amplified from the genomic DNA isolated from strain W3110. The following primers were used to amplify gene yjhG (EcoRI and HindIII restriction sites are underlined), 5′-CGGAATTCATGTCTGTTCGCAATATT (SEQ ID NO:19) and 5′-GCAAGCTTAATTCAGGTGTCTGGATG (SEQ ID NO:20). Gene yagF was amplified using the following primers (EcoRI and HindIII restriction sites are underlined), 5′-CGGAATTCGATGACCATTGAGAAAAT (SEQ ID NO:21) and 5′-GCAAGCTTCAACGATATATCTCAACT (SEQ ID NO:22). Localization of the yjhG and yagF PCR fragment between the EcoRI and HindIII sites of pJF118EH resulted in plasmid pWN7.270A and pWN7.272A, respectively. The following primers were used to amplify gene yjhH (EcoRI and BamHI restriction sites are underlined), 5′-CGGAATTCATGGGCTGGGATACAGAAAC (SEQ ID NO:23) and 5′-GCGGATCCTCAGACTGGTAAAATGCCCT (SEQ ID NO:24). Gene yagE was amplified using the following primers (EcoRI and BamHI restriction sites are underlined), 5′-CGGAATTCATGATTCAGCAAGGAGATC (SEQ ID NO:25) and 5′-TAGGATCCTTATCGTCCGGCTCAGCAA (SEQ ID NO:26). Localization of the yjhH and yagE PCR fragment between the EcoRI and BamHI sites of pJFI18EH resulted in plasmid pWN8.022A and pWN8.020A, respectively.

Plasmid pWN7.126B was derived from plasmid pWN5.238A. See, W. Niu et al., J. Am. Chem. Soc. 125:12998-12999 (2003). A 1.6-kb DNA fragment containing the serA gene was liberated from plasmid pRC1.55B by digestion with SmaI. Ligation of the serA locus with the ScaI-digested pWN5.238A resulted in plasmid pWN7.126B. Plasmid pWN9.068A was constructed for the purpose of generating E. coli WN13. The xdh gene from C. crescentus CB15 was amplified using the following primers with SphI restriction sites underlined, 5′-GCGCATGCATGTCCTCAGCCATCTATCC (SEQ ID NO:27) and 5′-GCGCATGCGATGACAGTTTTCTTAGGTC (SEQ ID NO:28). Insertion of the resulting PCR fragment into the SphI site of plasmid pKD3 resulted in pWN9.068A.

General enzymology. Cells were collected by centrifugation at 4,000 g and 4° C. Harvested cells were resuspended in the appropriate buffer and subsequently disrupted by two passages through a French press (16,000 psi or about 110.3 MPa). Cellular debris was removed by centrifugation at 48,000 g for 20 min. Protein concentrations were determined using the Bradford dye-binding method. See, M. M. Bradford, “A rapid and sensitive method for the quantification of microgram quantities of protein utilizing the principle of protein-dye binding,” Anal. Biochem. 72:248 (1976). Protein assay solution was purchased from Bio-Rad Laboratories, Inc. (Hercules, Calif., U.S.). Protein concentrations were determined by comparison to a standard curve prepared using bovine serum albumin.

D-Xylonic acid dehydratase activity was assayed according to procedures described previously. A. S. Dahms & A. Donald, “D-xylo-Aldonate dehydratase,” Methods in Enzymol. 90:302-305 (1982). The 2-keto acid formed during the reaction was quantified as its semicarbazone derivative. Resuspension buffer contained Tris-HCl (50 mM, pH 8.0) and MgCl₂(10 mM). Two solutions were prepared and incubated separately at 30° C. for 3 min. The first solution (150 μL) contained Tris-HCl (50 mM, pH 8.0), MgCl₂(10 mM) and an appropriate amount of cell lysate. The second solution (25 μL) contained potassium D-xylonate (0.1 M). After the two solutions were mixed (time=0), aliquots (30 μL) were removed at timed intervals and mixed with semicarbazide reagent (200 μL), which contained 1% (w/v) of semicarbazide hydrochloride and 0.9% (w/v) of sodium acetate in water. Following incubation at 30° C. for 15 min, each sample was diluted to 1 mL with H₂O. Precipitated protein was removed by microfugation. The absorbance of semicarbazone was measured at 250 nm. One unit of D-xylonate dehydratase activity was defined as the formation of 1 μmol of 2-keto acid per min at 30° C. A molar extinction coefficient of 10,200 M⁻¹ cm⁻¹ (250 nm) was used for 2-keto acid semicarbazone derivatives.

D-Xylose dehydrogenase was assayed using a modified procedure described previously. A. S. Dahms & J. Russo, “D-Xylose dehydrogenase,” Methods in Enzymol. 89(Pt. D):226-28 (1982). The resuspension buffer contained Tris-HCl (100 mM, pH 8.3). The enzymatic reaction (1 mL) contained Tris-HCl (100 mM, pH 8.3), NAD⁺ (2.5 mM), D-xylose (10 mM), and an appropriate amount of enzyme. The enzyme activity was measured spectrophotometrically by monitoring the formation of NADH at 340 nm. One unit of D-xylose dehydrogenase was defined as the formation of 1 μmol of NADH (c=6,220 M⁻¹ cm⁻¹) per min at 33° C.

The 3-deoxy-D-glycero-pentulosonic acid aldolase activity was measured according to a modified coupled-assay described previously. A. S. Dahms & A. Donald, “2-Keto-3-deoxy-D-xylonate aldolase (3-deoxy-D-pentulosonic acid aldolase),” Methods in Enzymol. 90 (Pt. E):269-72 (1982). Pyruvate liberated upon cleavage of the 2-keto acid was monitored in a reaction catalyzed by lactate dehydrogenase. The resuspension buffer contained HEPES (100 mM, pH 7.8). The assay solution (1 mL) contained HEPES (100 mM, pH 7.8), NADH (2 mM), lactate dehydrogenase (25 U), 3-deoxy-D,L-glycero-pentulosonic acid (5 mM), and an appropriate amount of enzyme. The background consumptions of NADH caused by NADH oxidase activity and possible endogenous pyruvate in the cell-free lysate were corrected by control experiments. One unit of 3-deoxy-D-glycero-pentulosonic acid aldolase activity was defined as the formation of 1 μmol of NAD⁺ (ε=6,220 M⁻¹ cm⁻¹) per min at room temperature.

Isolation of a partial gene sequence of P. fragi D-xylonic acid dehydratase. Cultivation of P. fragi for protein purification used a liquid medium (1 L) containing KH₂PO₄(4.5 g), Na₂HPO₄(4.7 g), NH₄Cl (1 g), CaCl₂(0.01 g), ferric ammonium citrate (0.1 g), MgSO₄(0.25 g), and corn steep liquor (0.1 g). See, e.g., R. Weimberg, J. Biol. Chem. 236:629-635 (1961). Growth of an inoculant was initiated by introduction of a single colony of P. fragi from a nutrient agar plate into 100 mL of the liquid medium containing D-xylose (0.25 g). The cells were cultured at 30° C. with agitation for 24 h. The resulting cell culture was transferred into a 2 L fermentor vessel that contained 1 L of the liquid medium with 10 g of D-xylose. Fermentor-controlled cultivation was carried out at 30° C., pH 6.5 with an impeller speed of 650 rpm for 48 h. Cells were harvested by centrifugation at 8,000 g and 4° C. for 10 min.

Buffers used for purification of D-xylonic acid dehydratase from P. fragi included buffer A: Tris-HCl (50 mM, pH 8.0), MgCl₂(2.5 mM), dithiothreitol (DTT) (1.0 mM), phenylmethylsulfonylfluoride (PMSF) (0.25 mM); buffer B: Tris-HCl (50 mM, pH 8.0), MgCl₂(2.5 mM), DTT (1.0 mM), PMSF (0.25 mM), NaCl (500 mM); buffer C: potassium phosphate (2.5 mM, pH 8.0), MgCl₂(2.5 mM), DTT (1.0 mM), PMSF (0.25 mM); buffer D: potassium phosphate (250 mM, pH 8.0), MgCl₂ (2.5 mM), DTT (1.0 mM), PMSF (0.25 mM); buffer E: Tris-HCl (50 mM, pH 8.0), MgCl₂(2.5 mM), DTT (1.0 mM), PMSF (0.25 mM), (NH₄)₂SO₄(1 M).

All protein purification manipulations were carried out at 4° C. D-Xylonic acid dehydratase specific activity was followed during the purification. P. fragi cells (150 g, wet weight) were resuspended in 250 mL of buffer A and disrupted by two passages through a French press cell at 16,000 psi (about 110.3 MPa). Cellular debris was removed by centrifugation (48,000 g, 20 min, 4° C.). The cell lysate was applied to a DEAE column (5×18 cm, packed with diethylaminoethyl Sepharose resin beads) equilibrated with buffer A. The column was washed with 1 L of buffer A followed by elution with a linear gradient (1.75 L+1.75 L, buffer A/buffer B). Fractions containing D-xylonic acid dehydratase were combined and concentrated to 100 mL. After dialysis against buffer C (3×1 L), the protein was loaded onto a hydroxyapatite column (2.5×35 cm) equilibrated with buffer C. The column was washed with 350 mL of buffer C and eluted with a linear gradient (850 mL+850 mL, buffer C/buffer D).

Fractions containing D-xylonic acid dehydratase were combined and concentrated to 30 mL. After dialysis against buffer E (3×300 mL), the protein solution was applied to a phenylsepharose column (2.5×15 cm) equilibrated with buffer E. The column was washed with 200 mL of buffer E followed by elution with a linear gradient (400 mL+400 mL, buffer E/buffer A). Fractions containing D-xylonic acid dehydratase were combined and concentrated to 15 mL. After dialysis against buffer A (3×150 mL), protein samples (15×0.1 mL) were loaded on a Resource 0 (6.4 mm×30 mm, 1 mL) column (from Amersham Biosciences, Piscataway, N.J., U.S.) equilibrated with buffer A. The column was washed with 25 mL of a 90:10 (v/v) mixture of buffer A and buffer B, and eluted with 20 column volumes of a linear gradient of NaCl (50 mM to 200 mM) in buffer A. Fractions containing D-xylonic acid dehydratase were combined and concentrated to 0.5 mL. After dialysis against buffer A (3×10 mL), the enzyme was quick frozen in liquid nitrogen and stored at about −80° C.

Trypsin digestion of the purified D-xylonic acid dehydratase, HPLC purification of the digestion products, and N-terminus peptide sequencing were carried out by the Macromolecular Structure Facility at Michigan State University. The DNA fragment encoding the partial P. fragi D-xylonic acid dehydratase was amplified from the genomic DNA of P. fragi using the following primers: 5′-CTGGARGAYTGGCARCGYGT (SEQ ID NO:29) and 5′-GTRTARTCYTCRGGRCCYTC (SEQ ID NO:30). The PCR product was cloned into pCRTOPO2.1 vector according to the manufacturer's instruction (Invitrogen Corp.). DNA sequence of the insert was determined using M13 forward and M13 reverse primers.

Purification and characterization of N-terminal 6×His-tagged D-xylose dehydrogenases. Single colony of E. coli DH5a/pWN9.044A and DH5a/pWN9.046A were respectively inoculated into 5 mL LB medium containing Ap. Inoculants were cultured at 37° C. with agitation overnight. Cells were subsequently transferred into 500 mL of LB containing Ap and grown at 37° C. with agitation. When the OD₆₀₀of the inoculants reached 0.4-0.6, the cell cultures were kept on ice for 10 min. IPTG solution was then added to the culture mediums to a final concentration of 0.5 mM. Cells were cultured for an additional 12 h at 30° C., then harvested by centrifugation at 4,000 g and 4° C. for 5 min. The harvested cells were resuspended in resuspension buffer containing Tris-HCl (100 mM, pH 8.0). Cell-free lysate was obtained as described in the general enzymology section. Purification of the 6×His-tagged D-xylose dehydrogenase using Ni-NTA resin followed protocols provided by the manufacture (Qiagen).

The cell-free lysate (16 mL) was mixed with 4 mL of Ni-NTA agarose resin (50% slurry (w/v)), and the mixture was stirred at 4° C. for one hour.

The lysate resin slurry was then transferred to a polypropylene column, and the column was washed with wash buffer (2×16 mL), which contains Tris-HCl (100 mM, pH 8.0), imidazole (20 mM), and NaCl (300 mM). The 6×His-tagged protein was eluted from the column by washing with elution buffer (2×4 mL), which contains Tris-HCl (100 mM, pH 8.0), imidazole (250 mM), and NaCl (300 mM). The eluted protein solution was dialyzed against cell resuspension buffer to remove imidazole and NaCl. Protein samples were analyzed using SDS-PAGE.

The pH dependence of the D-xylose dehydrogenases was measured between pH 4.4 and pH 9.0 at 33° C. using one of the following buffers: acetate (100 mM, pH 4.4-5.6), bis-Tris (100 mM, pH 5.6-7.5), or Tris-HCl (100 mM, pH 7.5-9.0). The substrate specificities of the enzymes were tested at 33° C. in Tris-HCl buffer (100 mM, pH 8.3) containing NAD⁺ (2.5 mM) and carbohydrate (50 mM). The Km and kcat values of the D-xylose dehydrogenases were obtained by analyzing experimental data using a nonlinear regression algorithm (Prism 4, GraphPad Software, Inc., San Diego, Calif., U.S.).

Random mutagenesis of E. coli. In vitro transposon mutagenesis of E. coli strain W3110 utilized the EZ::TN™ <R6Kyori/KAN-2> Tnp Transposome Kit (Epicentre) according to the protocols provided by the manufacture. The EZ:TN™ <R6Kyori/KAN-2> transposon-EZ:TN™ transposase complexes were introduced into electrocompetent E. coli W3110 by electroporation.

The electroporated cells were plated on LB plates containing kanamycin to select for mutants with transposon insertion into the chromosome. Colonies grown on these selection plates were further streaked out as pie plates. Single colonies from these pie plates were subjected to phenotypic analysis. Genomic DNAs isolated from W3110 mutants with desired phenotype were digested using EcoRI or BamHI. The chromosomal regions harboring the EZ::TN™ <R6KyorilKAN-2> transposon were rescued by electroporation of E. coli TRANSFORMAX EC100D pir⁺ electrocompetent cells (Epicentre) with the self-ligation mixture of the digested genomic DNA. The nucleotide sequences of the genomic DNA flanking the transposon element were determined by sequencing plasmids isolated from the recovered transformants on LB plates containing kanamycin. The DNA sequencing experiments utilized primers provided by the manufacturer (Epicentre).

Site-specific mutagenesis of yjhH and yagE genes. Disruption of the yjhH and yagE genes in E. coli W3110 utilized a chromosomal modification method described previously. See, K. A. Datsenko & B. L. Wanner, Proc. Natl. Acad. Sci. USA 97:6640-6645 (2000). In this method, E. coli strain that contained plasmid encoding the phage A red homologous recombination machinery was transformed with linear DNA fragment amplified using primers that were homologous to the targeted gene and template plasmid carrying antibiotic resistance gene flanked by FLP recognition target (FRT) sites. The DNA fragment used to disrupt the yjhH gene was amplified using the following primers from template pKD3: 5′-GTTGCCGACTTCCTGATTAATAAAGGGGTCGACGGGCTGTGTGTAGGCTGGA GCTGCTTCG (SEQ ID NO:31) and 5′-AACTGTGTTGATCATCGTACGCAAGTGACCAACGCTGTCGCATATGAATATCC TCCTTAGT (SEQ ID NO:32). The DNA fragment used to disrupt the yagE gene was amplified using the following primers from template pKD4: 5′-CCGGGAAACCATCGAACTCAGCCAGCACGCGCAGCACATATGAATATCCTCC TTAGT (SEQ ID NO:33) and 5′-GGATGGGCACCTTTGACGGTATGGATCATGCTGCGCGTGTAGGCTGGAGCTG CTTCG (SEQ ID NO:34). The PCR fragments were digested with DpnI and purified by electrophoresis. The purified DNA fragments were introduced into E. coli W3110/pKD46 by electroporation, respectively. Candidates of E. coli WN3 that contained yjhH::Cm^{R} on the chromosome were selected on LB plates containing chloramphenicol. Candidates of E. coli WN4 that contained yagE::Kan^{R} on the chromosome were selected on LB plates containing kanamycin. The correct genotype of the candidate strains was verified using PCRs. E. coli WN5 was generated by P1 phage-mediated transduction (see, J. H. Miller, ibid.) of yagE::Kan^{R} to the genome of WN3. Removal of the antibiotic resistance genes from the chromosome of E. coli WN5 followed the procedure described previously. See, K. A. Datsenko & B. L. Wanner, Proc. Natl. Acad. Sci. USA 97:6640-6645 (2000). The resulting strain was named as WN6.

Construction of E. coli host strains for the synthesis of D-1,2,4-butanetriol. E. coli W3110serA and WN7 were generated by following a previously described method (K. Li et al., Biotechnol. Bioeng. 64:61-73 (1999)) from strain W3110 and WN6, respectively. E. coli W3110xy/AB::xdh-Cm^{R} was constructed following the same procedure for the construction of strain WN3 and WN4. The DNA fragment used for chromosomal replacement was amplified from plasmid pWN9.068A using the following primers: 5′-TACGACATCATCCATCACCCGCGGCATTACCTGATTATGTCCTCAGCCATCTAT CCC (SEQ ID NO:35) and 5′-CAGAAGTTGCTGATAGAGGCGACGGAACGTTTCTCATATGAATATCCTCCTTA GT (SEQ ID NO:36). Candidates of strain W3110xylAB::xdh-Cm^{R} were selected on LB plate containing chloramphenicol. E. coli WN13 was generated by P1 phage-mediated transductions (see, J. H. Miller, ibid.)) of xylAB::xdh-Cre to the genome of WN7.

Fermentor-controlled cultivation conditions. Fermentations employed a 2.0 L working capacity B. Braun M2 culture vessel. Utilities were supplied by a B. Braun Biostat MD controlled by a DCU-3. Data acquisition utilized a Dell Optiplex Gs⁺ 5166M personal computer (PC) equipped with B. Braun MFCS/Win software (v1.1). Temperature, pH, and glucose feeding were controlled with PID control loops. Temperature was maintained at 33° C. for all fermentations. pH was maintained at 7.0 by addition of concentrated NH4OH or 2N H2SO4. Dissolved oxygen (D.O.) was measured using a Mettler-Toledo 12 mm sterilizable O₂ sensor fitted with an Ingold A-type O₂ permeable membrane. D.O. was maintained at 10% air saturation. The initial glucose concentration in the fermentation medium was 23.5 g/L.

Inoculants were started by introduction of a single colony picked from an agar plate into 5 mL of M9 medium. Cultures were grown at 37° C. with agitation at 250 rpm until they were turbid (about 24 h) and subsequently transferred to 100 mL of M9 medium. Cultures were grown at 37° C. and 250 rpm for an additional 10 h. The inoculant (OD600=1.0−3.0) was then transferred into the fermentation vessel and the batch fermentation was initiated (t=0 h).

Three staged methods were used to maintain D.O. concentrations at 10% air saturation during the fermentations. With the airflow at an initial setting of 0.06 L/L/min, the D.O. concentration was maintained by increasing the impeller speed from its initial set point of 50 rpm to its preset maximum of 940 rpm. With the impeller rate constant at 940 rpm, the mass flow controller then maintained the D.O. concentration by increasing the airflow rate from 0.06 L/L/min to a preset maximum of 1.0 L/L/min. At constant impeller speed and constant airflow rate, the D.O. concentration was finally maintained at 10% air saturation for the remainder of the fermentation by oxygen sensor-controlled glucose feeding. At the beginning of this stage, the D.O. concentration fell below 10% air saturation due to residual initial glucose in the medium. This lasted for approximately 10 min to 30 min before glucose (65% w/v) feeding commenced. The glucose feed PID control parameters were set to 0.0 s (off) for the derivative control (I_{D}) and 999.9 s (minimum control action) for the integral control (rᵢ). X_{P} was set to 950% to achieve a K_{c}of 0.1. IPTG stock solution (1.0 mL) was added to fermentation medium at 18 h. Solutions of D-xylose or potassium D-xylonate were added to the fermentation medium at 24 h, 30 h, 36 h, and 42 h.

Samples (5-10 mL) of fermentation broth were removed at the indicated timed intervals. Cell densities were determined by dilution of fermentation broth with water (1:100) followed by measurement of OD600. Dry cell weight of E. coli cells (g/L) was calculated using a conversion coefficient of 0.43 g/L/OD600. The remaining fermentation broth was centrifuged to obtain cell-free broth. The cell pellets were used for enzyme assays.

Metabolite characterizations. For the biosynthesis of 1,2,4-butanetriol, the concentration of 1,2,4-butanetriol in cell-free broth was quantified by GC analysis by following the method of W. Niu et al., J. Am. Chem. Soc. 125:12998-12999 (2003). The concentrations of other molecules in the cell-free broth were quantified by ¹H NMR. Solutions were concentrated to dryness under reduced pressure, concentrated to dryness one additional time from D₂O, and then redissolved in D₂O containing a known concentration of the sodium salt of 3-(trimethylsilyl)propionic-2,2,3,3-d₄ acid (TSP, Lancaster Synthesis Inc.). All ¹H NMR spectra were recorded on a Varian VXR-500 FT-NMR Spectrometer (500 MHz). Compounds were quantified by ¹H NMR using the following resonances: o-xylonic acid (δ 4.08, d, 1H); 3-deoxy-D-glycero-pentulosonic acid (δ 4.58, m, 1H).

To identify the biosynthetic byproducts in the fermentation medium, the cell-free fermentation broth was first applied to Dowex-I X4 resin (CI⁻ form). After washing with three column volumes of water, the column was eluted with ten column volumes of 0.1 M HCl. The flow-through and the wash fractions were combined and further applied to Dowex-50×8 resin (H⁺ form). After washing with three column volumes of water, the column was eluted with ten column volumes of 1 M HCl. Fractions obtained from the purification were neutralized and analyzed using ¹H NMR. Identification of 3-deoxy-D-glycero-pentulosonic acid and D-3,4-dihydroxy butanoic acid was done by comparing ¹H NMR spectra of purified samples with ¹H NMR spectra of authentic samples. To identify other molecules, the following NMR data were used: 3-deoxy-D-glycero-pentanoic acid, ¹H NMR (D₂O, 500 MHz, TSP, δ=0 ppm), δ 4.12 (dd, J=4, 8 Hz, 1H), 3.91 (m, 1H), 3.67 (dd, J=3, 12 Hz, 1H), 3.54 (dd, J=6, 12 Hz, 1H), 1.94 (ddd, J=1, 4, 14 Hz, 1H), 1.76 (ddd, J=1, 8, 15 Hz, 1H); (4S) 2-amino-4,5-dihydroxy pentanoic acid, ¹H NMR (D₂O, 500 MHz, TSP, δ=0 ppm), δ 4.01 (dd, J=5, 6 Hz, 1H), 3.89 (m, 1H), 3.64 (dd, J=4, 12 Hz, 1H), 3.55 (dd, J=6, 12 Hz, 1H), 2.04 (dd, J=5, 7 Hz, 2H).

Characterization of Host Cell Alcohol Dehydrogenase Activity. Screening efforts of candidate E. coli alcohol dehydrogenases was performed to identify which were the most active for reduction of 3,4-dihydroxy-D-butanal to D-1,2,4-butanetriol (Table 4). These efforts led to identification of AdhP (e.g., SEQ ID NO:38, encoded by SEQ ID NO:37).

TABLE 4
Screening of E. coli Dehydrogenases for
Reduction of 3,4-Dihydroxy-D-Butanal

| | | | | Crude Lysate Activity (U/mg) | |
|---|---|---|---|---|---|
| | | Size | | | 3,4-Di- |
| Entry | Gene | (kb) | Construct | Acetaldehyde | hydroxybutanal |
| 1 | adhP | 1.0 | DH5a/pML6.166 | 9.6 | 0.8 |
| 2 | adhE | 2.7 | DH5a/pML6.168 | 0.06 | 0.02 |
| 3 | yhdH | 1.0 | DH5a/pML6.259 | 0 | 0 |
| 4 | yiaY | 1.2 | DH5a/pML6.261 | 0.13 | 0.03 |
| 5 | ydjO | 0.8 | DH5a/pML6.263 | 0 | 0 |

To further charactrerize the role of AdhP, e.g., to determine if it was the sole dehydrogenase responsible for the reduction of 3,4-dihydroxy-D-butanal in D-1,2,4-butanetriol-synthesizing E. coli constructs, the adhP gene was deleted in KIT10 (Table 5) and the impact on this deletion on biosynthesis of D-1,2,4-butanetriol appraised (Table 6). Underlining in Table 5 shows changes to the host cell genotype.

TABLE 5
Strains Used to Evaluate adhP Inactivation and D-1,2,4-Butanetriol Biosynthesis.

| Construct | Genotype |
|---|---|
| WN13/pWN7.126B | E. coliW3110serAΔyjhHΔyagExylAB::xdh-Cm^{R}/serA, lacl^{Q} P_{tac}mdlC |
| WN10/pWN7.126B | E. coliW3110serAΔyjhHΔyagExylAB::xdh-Cm^{R}ΔadhP/serA, lacl^{Q} P_{tac}mdlC |

TABLE 6
Impact of adhP Inactivation on D-1,2,4-Butanetriol Biosynthesis.

| | Titer, g/L | | | | |
|---|---|---|---|---|---|
| Construct | 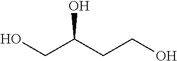 A | 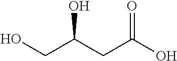 B | 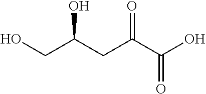 | A/B (mol/mol) | Yield of A (%) |
| WN13/pWN7.126B | 10.2 | 4.6 | 0 | 2.2 | 50 |
| WN10/pWN7.126B | 6.5 | 5.3 | 7.2 | 1.2 | 31 |

These tests showed that formation of D-1,2,4-butanetriol decreased (Table 6) and the ratio of 3,4-dihydroxy-D-butyric acid to D-1,2,4-butanetriol increased (Table 6) upon deletion of adhP. These experiments establish that adhP likely plays a role in the reduction of 3,4-dihydroxy-D-butanal in D-1,2,4-butanetriol-synthesizing E. coli constructs, but AdhP is not the only dehydrogenase involved in this reduction, as others exhibit the same activity to a lesser degree.

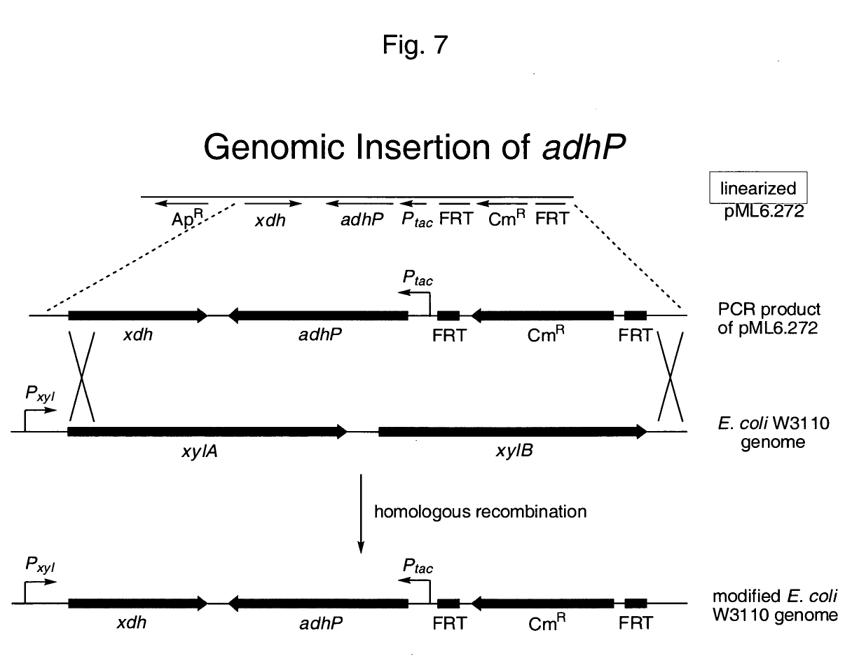

Effects of AdhP Alcohol Dehydrogenase Overexpression. In order to asses whether or not AdhP overexpression could decrease the amount of 3,4-dihydroxy-D-butyric acid and increase the amount of D-1,2,4-butanetriol, assays were performed using either plasmid-localized expression of adhP behind a P_{tac} promoter (E. coli WN13/pML6.195, Table 7) or genomic insertion of adhP behind the P_{xyl} promoter (E. coli KIT4/pWN7.126B, Table 7). Genomic insertion was performed according to the strategy illustrated in FIG. 7. Underlining in Table 7 shows changes to the host cell genotype.

TABLE 7
Strains Used to Evaluate adhP Overexpression and D-1,2,4-Butanetriol Biosynthesis.

| Construct | Genotype |
|---|---|
| WN13/pWN7.126B | E. coliW3110serAΔyjhHΔyagExylAB::xdh-Cm^{R}/serA, lacl^{Q} P_{tac}mdlC |
| KIT10/pWN7.126B | E. coliW3110serAΔyjhHΔyagExylAB::xdh-Cm^{R}ΔadhP/serA, lacl^{Q} P_{tac}mdlC |
| WN13/pML6.195 | E. coliW3110serAΔyjhHΔyagExylAB::xdh-Cm^{R}/serA, lacl^{Q} P_{tac}mdlC P_{tac}adhP |
| KIT4/pWN7.126B | E. coliW3110serAΔyjhHΔyagExylAB::xdh-ΔadhP-P_{tac}/serA, lacl^{Q} P_{tac}mdlC |

Results are presented in Table 8. These results indicate that genomic insertion was the most successful strategy (Table 8).

TABLE 8
Impact of adhP Overexpression on D-1,2,4-Butanetriol Biosynthesis.

| | Titer, g/L | | | | |
|---|---|---|---|---|---|
| Construct | 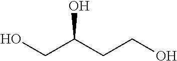 A | 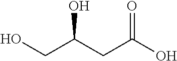 B | 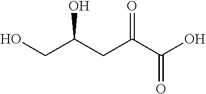 | A/B (mol/mol) | Yield of A (%) |
| WN13/pWN7.126B | 10.2 | 4.6 | 0 | 2.2 | 50 |
| KIT10/pWN7.126B | 6.5 | 5.3 | 7.2 | 1.2 | 31 |
| WN13/pML6.195 | 4.6 | 4.5 | 10.0 | 1 | 22 |
| KIT4/pWN7.126B | 11.5 | 4.5 | 0 | 2.6 | 55 |

Effects of Inactivation of Enzymes Competing for a Key Intermediate in the Novel Butanetriol Biosynthesis Pathway. Reduction of intermediate 3-deoxy-D-glycero-pentulosonic acid to the byproduct, 3-deoxy-D-glycero-pentanoic acid, is postulated to be responsible for lowering yields and concentrations of D-1,2,4-butanetriol biosynthesized by the novel pathway hereof. See reaction (e) in FIG. 5d. Two 2-keto acid dehydrogenases, YiaE (SEQ ID NO:40, encoded by SEQ ID NO:39) and YcdW (SEQ ID NO:42, encoded by SEQ ID NO:41), have been identified to catalyze this reduction of 3-deoxy-D-glycero-pentulosonic acid. To determine if improvement in butanetriol yield could be obtained, genomic inactivation of yiaE and ycdW was performed (E. coli KIT18/pWN7.126B, Table 9).

TABLE 9
Strains Used to Evaluate yiaE and ycdW Knockouts on D-1,2,4-butanetriol Biosynthesis.

| Construct | Genotype |
|---|---|
| WN13/pWN7.126B | E. coliW3110serAΔyjhHΔyagExylAB::xdh-Cm^{R}/serA, lacl^{Q} P_{tac}mdlC |
| KIT18/pWN7.126B | E. coliW3110serAΔyjhHΔyagExylAB::xdh-adhP-P_{tac}ΔyiaEΔycdW/serA, lacl^{Q} P_{tac}mdlC |

The biosynthesis of D-1,2,4-butanetriol from D-xlyose was determined, with monitoring of byproduct formation (Table 10).

TABLE 10
Impact of yiaE and ycdW Knockouts on D-1,2,4-butanetriol Biosynthesis

| | | | Titer, g/L | | |
|---|---|---|---|---|---|
| Construct | X (g) | t (h) | 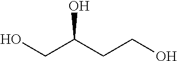 A | 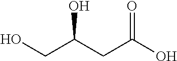 B | 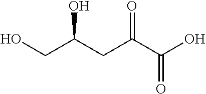 |
| WN13/pWN7.126B | 30 | 48 | 10.2 | 4.6 | 0 |
| KIT18/pWN7.126B | 30 | 48 | 11.2 | 3.9 | 0 |
| KIT18/pWN7.126B | 50 | 48 | 16.5 | 4.9 | 3 |
| KIT18/pWN7.126B | 50 | 54 | 18.0 | 5.2 | 0 |

| | | | Titer, g/L | | | |
|---|---|---|---|---|---|---|
| Construct | X (g) | t (h) | 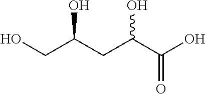 | 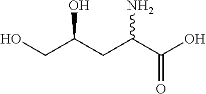 | A/B | % A |
| WN13/pWN7.126B | 30 | 48 | 5.1 | 3.8 | 2.2 | 50 |
| KIT18/pWN7.126B | 30 | 48 | 2.9 | 5.3 | 2.9 | 31 |
| KIT18/pWN7.126B | 50 | 48 | 5.4 | 6 | 3.4 | 22 |
| KIT18/pWN7.126B | 50 | 54 | 5.5 | 5.9 | 3.5 | 55 |

This data shows that gene inactivation decreases the concentration of the byproduct, 3-deoxy-D-glycero-pentanoic acid, and increase the concentration and yield of biosynthesized D-1,2,4-butanetriol. E. coli KIT18/pWN7.126B was also observed to continue growing for a longer period of time relative to E. coli WN13/pWN7.126B. This allowed a larger amount of D-xylose (50 g versus 30 g, Table 10) to be added and consumed, which resulted in a pronounced increase in the concentration of D-1,2,4-butanetriol. Increasing the amount of D-xylose added to cultures of E. coli KIT18/pWN7.126B also resulted in a pronounced increase in the ratio of D-1,2,4-butanetriol biosynthesized relative to 3,4-dihydroxy-D-butyric acid (Table 10).

In summary, these results show that the biosynthesis of butanetriol by a novel pathway hereof is improved by adding a second copy, preferably a second genomic copy or copies, of a 3,4-dihydroxy-D-butanal-utilizing alcohol dehydrogenase, such as adhP (or adhE or yiaY). In addition, these results show that inactivation of 2-keto acid dehydrogenase activity, e.g., as by inactivating yiaE and ycdW, independently improves butanetriol production. When done in combination, these two added elements provide a surprising 80% increase in the concentration of D-1,2,4-butanetriol biosynthesized from D-xylose.

### Sequence Listing Free Text

Coding sequence for Burkholderia fungorum LB400 RBU11704 xylose dehydrogenase
Coding sequence for Caulobacter crescentus CB15 RC001012 xylose dehydrogenase
Coding sequence for E. coli yjhG xylonate dehydratase
Coding sequence for Escherichia coli yagF xylonate dehydratase
Coding sequence for Pseudomonas fragi ATCC 4973 xylonate dehydratase fragment.
n is a, c, g, or t
Coding sequence for E. coli yjhH 3-deoxy-D-glycero-pentulosonate aldolase
Putative initiator codon
Alternative initiator codon
Alternative coding sequence for E. coli yjhH 3-deoxy-D-glycero-pentulosonate aldolase polypeptide
Putative initiator Met
E. coli yjhH 3-deoxy-D-glycero-pentulosonate aldolase polypeptide
Alternative E. coli yjhH 3-deoxy-D-glycero-pentulosonate aldolase polypeptide.
Alternative initiator Met
Coding sequence for E. coli yagE 3-deoxy-D-glycero-pentulosonate aldolase
Forward amplification primer for Burkholderia fungorum LB400 D-xylose dehydrogenase gene (RBU11704)
Reverse amplification primer for B. fungorum LB400 D-xylose dehydrogenase gene (RBU11704)
Forward amplification primer for Caulobacter crescentus CB15 D-xylose dehydrogenase gene (RC001012)
Reverse amplification primer for C. crescentus CB15 D-xylose dehydrogenase gene (RC001012)
Forward amplification primer for E. coli W3110 D-xylonate dehydratase gene (yjhG)
Reverse amplification primer for E. coli W3110 D-xylonate dehydratase gene (yjhG)
Forward amplification primer for E. coli W3110 D-xylonate dehydratase gene (yagF)
Reverse amplification primer for E. coli W3110 D-xylonate dehydratase gene (yagF)
Forward amplification primer for E. coli W3110 3-deoxy-D-glycero-pentulosonate aldolase gene (yjhH)
Reverse amplification primer for E. coli W3110 3-deoxy-D-glycero-pentulosonate aldolase gene (yjhH)
Forward amplification primer for E. coli W3110 3-deoxy-D-glycero-pentulosonate aldolase gene (yagE)
Reverse amplification primer for E. coli W3110 3-deoxy-D-glycero-pentulosonate aldolase gene (yagE)
Forward amplification primer for C. crescentus CB15 D-xylose dehydrogenase gene, for construction of plasmid pWN9.068A
Reverse amplification primer for C. crescentus CB15 D-xylose dehydrogenase gene, for construction of plasmid pWN9.068A
Forward amplification primer for Pseudomonas fragi xylonate dehydratase gene
Reverse amplification primer for Pseudomonas fragi xylonate dehydratase gene
Forward amplification primer for the DNA fragment for use in disrupting the E. coli genomic 3-deoxy-D-glycero-pentulosonate aldolase gene (yjhH)
Reverse amplification primer for the DNA fragment for use in disrupting the E. coli genomic 3-deoxy-D-glycero-pentulosonate aldolase gene (yjhH)
Forward amplification primer for the DNA fragment for use in disrupting the E. coli genomic 3-deoxy-D-glycero-pentulosonate aldolase gene (yagE)
Reverse amplification primer for the DNA fragment for use in disrupting the E. coli genomic 3-deoxy-D-glycero-pentulosonate aldolase gene (yagE)
Forward amplification primer for the DNA fragment for use in inserting xdh into the E. coli genomic DNA
Reverse amplification primer for the DNA fragment for use in inserting xdh into the E. coli genomic DNA
Coding Sequence for E. coli AdhP alcohol dehydrogenase, from GenBank 000096
AdhP 1-propanol-preferring, two-zinc-ion-containing alcohol dehydrogenase (Genbank Accession No. AAC74551) of IUBMB EC 1.1.1.1
H24-V131 constitutes an alcohol dehydrogenase GroES-like domain belonging to PfamA Accession No. PF08240
Conserved Cys binding to catalytic zinc ion
G57-V71 constitutes a Zinc-Containing Alcohol Dehydrogenase Signature Domain classified under ProSite Accession No. PS00059 whose consensus pattern is “G-H-E-x-{EL}-G-{AP}-x(4)-[GA]-x(2)-[IVSAC]”
Conserved H is binding to catalytic zinc ion
Conserved Cys binding to second zinc ion
Conserved Cys binding to second zinc ion
Conserved Cys binding to second zinc ion
Conserved Cys binding to second zinc ion
Conserved Cys binding to catalytic zinc ion
P161-E299 constitutes a zinc-binding alcohol dehydrogenase domain belonging to PfamA Accession No. PF00107
G172-L260 constitutes a nucleotide-binding motif belonging to ProSite Accession No. PS50193 for “SAM (and some other nucleotide) Binding Motif”
Coding Sequence for E. coli yiaE 2-keto acid dehydrogenase, from GenBank AE005174
YiaE 2-keto acid dehydrogenase (Genbank Accession No. AAG58702)
Coding Sequence for E. coli ycdW 2-Keto acid Dehydrogenase, from GenBank AP009048
YcdW 2-Keto acid Dehydrogenase (Genbank Accession No. BAA35814)
Coding Sequence for P. putida mdIC 2-keto acid decarboxylase, from GenBank AY143338
MdIC 2-keto acid decarboxylase (Genbank Accession No. AAC15502)

What is claimed is:

1. A process for preparing D-1,2,4-butanetriol, comprising:
(A) providing
(1) a recombinant cellular entity containing a 1,2,4-butanetriol biosynthetic enzyme system that comprises (a) a D-xylose dehydrogenase, (b) a D-xylonic acid dehydratase, (c) a 2-keto acid decarboxylase, and (d) an alcohol dehydrogenase, wherein the cellular entity is one that has been manipulated to inhibit or inactivate (e) a 3-deoxy-D-glycero-pentulosonic acid aldolase polypeptide or nucleic acid thereof, (f) a 2-keto-acid dehydrogenase polypeptide or nucleic acid thereof, or (g) both (e) and (f); and
(2) a xylose source that is capable, under conditions in which the enzyme system can operate to produce 1,2,4-butanetriol, of providing D-xylose to the D-xylose dehydrogenase enzyme; and
(B) placing the cellular entity and the xylose source under conditions in which the enzyme system can produce 1,2,4-butanetriol from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate,
(3) the 2-keto acid decarboxylase to convert resulting 3-deoxy-D-glycero-pentulosonate to 3,4-dihydroxy-D-butanal, and
(4) the alcohol dehydrogenase to convert resulting 3,4-dihydroxy-D-butanal to D-1,2,4-butanetriol;
thereby preparing D-1,2,4-butanetriol.

2. A process for preparing D-1,2,4-butanetriol, comprising:
(A) providing
(1) a recombinant cellular entity containing a 1,2,4-butanetriol biosynthetic enzyme system that comprises (a) a D-xylose dehydrogenase comprising the amino acid sequence of any one of SEQ ID NO:2, SEQ ID NO:4, or a conservative-substituted variant of or homologous polypeptide to SEQ ID NO:2 or SEQ ID NO:4 and having D-xylose dehydrogenase activity, (b) a D-xylonic acid dehydratase, (c) a 2-keto acid decarboxylase, and (d) an alcohol dehydrogenase, and
(2) a xylose source that is capable, under conditions in which the enzyme system can operate to produce 1,2,4-butanetriol, of providing D-xylose to the D-xylose dehydrogenase enzyme; and
(B) placing the cellular entity and the xylose source under conditions in which the enzyme system can produce 1,2,4-butanetriol from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate,
(3) the 2-keto acid decarboxylase to convert resulting 3-deoxy-D-glycero-pentulosonate to 3,4-dihydroxy-D-butanal, and
(4) the alcohol dehydrogenase to convert resulting 3,4-dihydroxy-D-butanal to D-1,2,4-butanetriol;
thereby preparing D-1,2,4-butanetriol.

3. A process for preparing D-1,2,4-butanetriol, comprising:
(A) providing
(1) a recombinant cellular entity containing a 1,2,4-butanetriol biosynthetic enzyme system that comprises
(a) a D-xylose dehydrogenase,
(b) a D-xylonic acid dehydratase comprising
(i) the amino acid sequence of any one of SEQ ID NO:6, SEQ ID NO:8, or a conservative-substituted variant of or homologous polypeptide to SEQ ID NO:6 or SEQ ID NO:8 and having D-xylonate dehydratase activity, or
(ii) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or a conservative-substituted variant thereof or homologous polypeptide thereto,
(c) a 2-keto acid decarboxylase, and
(d) an alcohol dehydrogenase, and
(2) a xylose source that is capable, under conditions in which the enzyme system can operate to produce 1,2,4-butanetriol, of providing D-xylose to the D-xylose dehydrogenase enzyme; and
(B) placing the cellular entity and the xylose source under conditions in which the enzyme system can produce 1,2,4-butanetriol from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate,
(3) the 2-keto acid decarboxylase to convert resulting 3-deoxy-D-glycero-pentulosonate to 3,4-dihydroxy-D-butanal, and
(4) the alcohol dehydrogenase to convert resulting 3,4-dihydroxy-D-butanal to D-1,2,4-butanetriol,
thereby preparing D-1,2,4-butanetriol.

4. A process for preparing D-1,2,4-butanetriol, comprising:
(A) providing
(1) a recombinant cellular entity containing a 1,2,4-butanetriol biosynthetic enzyme system that comprises
(a) a D-xylonic acid dehydratase comprising
(i) the amino acid sequence of any one of SEQ ID NO:6, SEQ ID NO:8, or a conservative-substituted variant of or homologous polypeptide to SEQ ID NO:6 or SEQ ID NO:8 and having D-xylonate dehydratase activity, or
(ii) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or a conservative-substituted variant thereof or homologous polypeptide thereto,
(b) a 2-keto acid decarboxylase, and
(c) an alcohol dehydrogenase, and
(2) a xylonate source that is capable, under conditions in which the enzyme system can operate to produce 1,2,4-butanetriol, of providing D-xylonate to the D-xylonic acid dehydratase enzyme; and
(B) placing the cellular entity and the xylonate source under conditions in which the enzyme system can produce 1,2,4-butanetriol from D-xylonate, and in which the xylonate source provides D-xylonate to the D-xylonic acid dehydratase enzyme, the enzyme system operating under the conditions by action of
(1) the D-xylonic acid dehydratase to convert resulting D-xylonate to 3-deoxy-D-glycero-pentulosonate,
(2) the 2-keto acid decarboxylase to convert resulting 3-deoxy-D-glycero-pentulosonate to 3,4-dihydroxy-D-butanal, and
(3) the alcohol dehydrogenase to convert resulting 3,4-dihydroxy-D-butanal to D-1,2,4-butanetriol;
thereby preparing D-1,2,4-butanetriol.

5. A process for preparing D-1,2,4-butanetriol, comprising:
(A) providing
(1) a recombinant cellular entity containing a 1,2,4-butanetriol biosynthetic enzyme system that comprises (a) a D-xylonic acid dehydratase, (b) a 2-keto acid decarboxylase, and (c) an alcohol dehydrogenase, wherein the cellular entity is one that has been manipulated to inhibit or inactivate (d) a 3-deoxy-D-glycero-pentulosonic acid aldolase polypeptide or nucleic acid thereof, (e) a 2-keto-acid dehydrogenase polypeptide or nucleic acid thereof, or (f) both (d) and (e); and
(2) a xylonate source that is capable, under conditions in which the enzyme system can operate to produce 1,2,4-butanetriol, of providing D-xylonate to the D-xylonic acid dehydratase enzyme; and
(B) placing the cellular entity and the xylose source under conditions in which the enzyme system can produce 1,2,4-butanetriol from D-xylonic acid, and in which the xylonate source provides D-xylonate to the D-xylonate dehydratase enzyme, the enzyme system operating under the conditions by action of
(1) the D-xylonic acid dehydratase to convert D-xylonate to 3-deoxy-D-glycero-pentulosonate,
(2) the 2-keto acid decarboxylase to convert resulting 3-deoxy-D-glycero-pentulosonate to 3,4-dihydroxy-D-butanal, and
(3) the alcohol dehydrogenase to convert resulting 3,4-dihydroxy-D-butanal to D-1,2,4-butanetriol;
thereby preparing D-1,2,4-butanetriol.

6. The process according to claim 1, wherein the recombinant cellular entity comprises a microbial or plant cell that contains the enzyme system.

7. (canceled)

8. The process according to claim 1, wherein the xylose source comprises D-xylose, a carbon source from which D-xylose can be anabolically synthesized under said conditions, or a D-xylose-residue-containing polymer from which D-xylose residues can be hydrolyzed under said conditions.

9-12. (canceled)

13. A process for preparing 1,2,4-butanetriol trinitrate, comprising
(A) providing D-1,2,4-butanetriol prepared by a process according to claim 1, and a nitrating agent, and
(B) contacting the D-1,2,4-butanetriol with the nitrating agent under conditions in which the nitrating agent can nitrate the D-1,2,4-butanetriol, thereby preparing 1,2,4-butanetriol trinitrate.

14. (canceled)

15. A D-xylose dehydrogenase comprising the amino acid sequence of any one of SEQ ID NO:2, SEQ ID NO:4, or a conservative-substituted variant of or homologous polypeptide to SEQ ID NO:2 or SEQ ID NO:4 and having D-xylose dehydrogenase activity.

16. (canceled)

17. Nucleic acid encoding a D-xylose dehydrogenase according to claim 15, wherein the nucleic acid comprises the base sequence of any one of SEQ ID NO:1, SEQ ID NO:3, or a homologous polynucleotide to SEQ ID NO:1 or SEQ ID NO:3.

18. (canceled)

19. The nucleic acid according to claim 17, wherein the nucleic acid is a plasmid.

20. (canceled)

21. A D-xylonic acid dehydratase comprising the amino acid sequence of any one of: SEQ ID NO:6; SEQ ID NO:8; a conservative-substituted variant of or homologous polypeptide to SEQ ID NO:6 or SEQ ID NO:8; a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end; or a conservative-substituted variant of or homologous polypeptide to the P. fragi D-xylonate dehydratase amino acid sequence.

22. (canceled)

23. Nucleic acid encoding a D-xylonic acid dehydratase according to claim 21, wherein the nucleic acid comprises the base sequence of any one of SEQ ID NO:1, SEQ ID NO:3, or a homologous polynucleotide to SEQ ID NO:1 or SEQ ID NO:3.

24. (canceled)

25. The nucleic acid according to claim 23, wherein the nucleic acid is a plasmid.

26. (canceled)

27. An isolated or recombinant 1,2,4-butanetriol biosynthetic enzyme system that comprises:
(A) a D-xylose dehydrogenase comprising the amino acid sequence of any one of SEQ ID NO:2, SEQ ID NO:4, or a conservative-substituted variant of or homologous polypeptide to SEQ ID NO:2 or SEQ ID NO:4 and having D-xylose dehydrogenase activity,
(B) a D-xylonic acid dehydratase,
(C) a 2-keto acid decarboxylase, and
(D) an alcohol dehydrogenase,
the enzyme system being capable of catalyzing the conversion of D-xylose to D-1,2,4-butanetriol.

28. An isolated or recombinant 1,2,4-butanetriol biosynthetic enzyme system that comprises:
(A) a D-xylose dehydrogenase,
(B) a D-xylonic acid dehydratase comprising
(1) the amino acid sequence of any one of SEQ ID NO:6, SEQ ID NO:8, or a conservative-substituted variant of or homologous polypeptide to SEQ ID NO:6 or SEQ ID NO:8 and having D-xylonate dehydratase activity, or
(2) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or a conservative-substituted variant thereof or homologous polypeptide thereto,
(C) a 2-keto acid decarboxylase, and
(D) an alcohol dehydrogenase,
the enzyme system being capable of catalyzing the conversion of D-xylose to D-1,2,4-butanetriol.

29. An isolated or recombinant 2,4-butanetriol biosynthetic enzyme system that comprises
(A) a D-xylonic acid dehydratase comprising
(1) the amino acid sequence of any one of SEQ ID NO:6, SEQ ID NO:8, or a conservative-substituted variant of or homologous polypeptide to SEQ ID NO:6 or SEQ ID NO:8 and having D-xylonate dehydratase activity, or
(2) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or a conservative-substituted variant thereof or homologous polypeptide thereto,
(B) a 2-keto acid decarboxylase, and
(C) an alcohol dehydrogenase,
the enzyme system being capable of catalyzing the conversion of D-xylonate to D-1,2,4-butanetriol.

30. A recombinant cellular entity that comprises an enzyme system according to claim 27, wherein the cellular entity comprises a single cell that contains the enzyme system.

31. (canceled)

32. The recombinant cellular entity according to claim 31, wherein the cell is a recombinant DgPu⁻ cell.

33. A 3-deoxy-D-glycero-pentulosonate aldolase knock-out vector comprising a polynucleotide containing a base sequence from any one of SEQ ID NO:11, SEQ ID NO:13, or nt55-319 of SEQ ID NO:11, wherein the vector is capable of inserting into or recombining with a genomic copy of a 3-deoxy-D-glycero-pentulosonate aldolase gene in such a manner as to inactivate the gene or its encoded aldolase.

34. A recombinant cell that is DgPu⁻ (3-deoxy-D-glycero-pentulosonate aldolase “minus”), or KAD⁻ (2-keto-acid dehydrogenase “minus”), or both DgPu⁻ and KAD⁻.

35. A process for preparing 3-deoxy-D-glycero-pentanoic acid, comprising:
(A) providing
(1) a recombinant cellular entity containing a 3-deoxy-D-glycero-pentanoic acid biosynthetic enzyme system that comprises (a) a D-xylose dehydrogenase, (b) a D-xylonic acid dehydratase, and (c) a 2-keto-acid reductase,
and
(2) a xylose source that is capable, under conditions in which the enzyme system can operate to produce 3-deoxy-D-glycero-pentanoic acid, of providing D-xylose to the D-xylose dehydrogenase enzyme; and
(B) placing the cellular entity and the xylose source under conditions in which the enzyme system can produce 3-deoxy-D-glycero-pentanoic acid from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate, and
(3) the 2-keto acid dehydrogenase (reductase) to convert resulting 3-deoxy-D-glycero-pentulosonate to 3-deoxy-D-glycero-pentanoic acid,
thereby preparing 3-deoxy-D-glycero-pentanoic acid.

36. A process for preparing 3-deoxy-D-glycero-pentanoic acid, comprising:
(A) providing
(1) a recombinant cellular entity containing a 3-deoxy-D-glycero-pentanoic acid biosynthetic enzyme system that comprises (a) a D-xylonic acid dehydratase, and (b) a 2-keto-acid reductase,
and
(2) a xylonate source that is capable, under conditions in which the enzyme system can operate to produce 3-deoxy-D-glycero-pentanoic acid, of providing D-xylonate to the D-xylonate dehydratase enzyme; and
(B) placing the cellular entity and the xylose source under conditions in which the enzyme system can produce 3-deoxy-D-glycero-pentanoic acid from D-xylonate, and in which the xylonate source provides D-xylonate to the D-xylonate dehydratase enzyme, the enzyme system operating under the conditions by action of
(1) the D-xylonic acid dehydratase to convert D-xylonate to 3-deoxy-D-glycero-pentulosonate, and
(2) the 2-keto acid dehydrogenase (reductase) to convert resulting 3-deoxy-D-glycero-pentulosonate to 3-deoxy-D-glycero-pentanoic acid,
thereby preparing 3-deoxy-D-glycero-pentanoic acid.

37. (canceled)

38. A process for preparing D-3,4-dihydroxy-butanoic acid, comprising:
(A) providing
(1) a recombinant cellular entity containing a D-3,4-dihydroxy-butanoic acid biosynthetic enzyme system that comprises (a) a D-xylose dehydrogenase, (b) a D-xylonic acid dehydratase, and (c) a 2-keto-acid decarboxylase, and (d) an aldehyde dehydrogenase, and
(2) a xylose source that is capable, under conditions in which the enzyme system can operate to produce D-3,4-dihydroxy-butanoic acid, of providing D-xylose to the D-xylose dehydrogenase enzyme; and
(B) placing the cellular entity and the xylose source under conditions in which the enzyme system can produce D-3,4-dihydroxy-butanoic acid from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate, and
(3) the 2-keto acid decarboxylase to convert resulting 3-deoxy-D-glycero-pentulosonate to 3,4-dihydroxy-D-butanal, and
(4) the aldehyde dehydrogenase to convert resulting 3,4-dihydroxy-D-butanal to D-3,4-dihydroxy-butanoic acid,
thereby preparing D-3,4-dihydroxy-butanoic acid.

39. A process for preparing D-3,4-dihydroxy-butanoic acid, comprising:
(A) providing
(1) a recombinant cellular entity containing a D-3,4-dihydroxy-butanoic acid biosynthetic enzyme system that comprises (a) a D-xylonic acid dehydratase, and (b) 2-keto-acid decarboxylase, and (c) an aldehyde dehydrogenase, and
(2) a xylonate source that is capable, under conditions in which the enzyme system can operate to produce D-3,4-dihydroxy-butanoic acid, of providing D-xylonate to the D-xylonate dehydratase enzyme; and
(B) placing the cellular entity and the xylonate source under conditions in which the enzyme system can produce D-3,4-dihydroxy-butanoic acid from D-xylonic acid, and in which the xylonate source provides D-xylonate to the D-xylonate dehydratase enzyme, the enzyme system operating under the conditions by action of
(1) the D-xylonic acid dehydratase to convert D-xylonate to 3-deoxy-D-glycero-pentulosonate, and
(2) the 2-keto acid decarboxylase to convert resulting 3-deoxy-D-glycero-pentulosonate to 3,4-dihydroxy-D-butanal, and
(3) the aldehyde dehydrogenase to convert resulting 3,4-dihydroxy-D-butanal to D-3,4-dihydroxy-butanoic acid,
thereby preparing D-3,4-dihydroxy-butanoic acid.

40. (canceled)

41. A process for preparing (4S)-2-amino-4,5-dihydroxy pentanoic acid, comprising:
(A) providing
(1) a recombinant cellular entity containing a 3-deoxy-D-glycero-pentanoic acid biosynthetic enzyme system that comprises (a) a D-xylose dehydrogenase, (b) a D-xylonic acid dehydratase, and (c) a 2-keto acid transaminase,
and
(2) a xylose source that is capable, under conditions in which the enzyme system can operate to produce (4S)-2-amino-4,5-dihydroxy pentanoic acid, of providing D-xylose to the D-xylose dehydrogenase enzyme; and
(B) placing the cellular entity and the xylose source under conditions in which the enzyme system can produce (4S)-2-amino-4,5-dihydroxy pentanoic acid from D-xylose, and in which the xylose source provides D-xylose to the D-xylose dehydrogenase enzyme, the enzyme system operating under the conditions by action of
(1) the D-xylose dehydrogenase enzyme to convert D-xylose to D-xylonate,
(2) the D-xylonic acid dehydratase to convert resulting D-xylonate to 3-deoxy-D-glycero-pentulosonate, and
(3) the 2-keto acid transaminase to convert resulting 3-deoxy-D-glycero-pentulosonate to (4S)-2-amino-4,5-dihydroxy pentanoic acid,
thereby preparing (4S)-2-amino-4,5-dihydroxy pentanoic acid.

42. A process for preparing (4S)-2-amino-4,5-dihydroxy pentanoic acid, comprising:
(A) providing
(1) a recombinant cellular entity containing a 3-deoxy-D-glycero-pentanoic acid biosynthetic enzyme system that comprises (a) a D-xylonic acid dehydratase, and (b) a 2-keto acid transaminase,
and
(2) a xylonate source that is capable, under conditions in which the enzyme system can operate to produce (4S)-2-amino-4,5-dihydroxy pentanoic acid, of providing D-xylonate to the D-xylonate dehydratase enzyme; and
(B) placing the cellular entity and the xylose source under conditions in which the enzyme system can produce (4S)-2-amino-4,5-dihydroxy pentanoic acid from D-xylonic acid, and in which the xylonate source provides D-xylonate to the D-xylonate dehydratase enzyme, the enzyme system operating under the conditions by action of
(1) the D-xylonic acid dehydratase to convert D-xylonate to 3-deoxy-D-glycero-pentulosonate, and
(2) the 2-keto acid transaminase to convert resulting 3-deoxy-D-glycero-pentulosonate to (4S)-2-amino-4,5-dihydroxy pentanoic acid, thereby preparing (4S)-2-amino-4,5-dihydroxy pentanoic acid.

43-45. (canceled)

46. An isolated or recombinant 3-deoxy-D-glycero-pentanoic acid biosynthetic enzyme system that comprises: (A) a D-xylose dehydrogenase, (B) a D-xylonic acid dehydratase, and (C) a 2-keto-acid reductase, the enzyme system being capable of catalyzing the conversion of D-xylose to 3-deoxy-D-glycero-pentanoic acid.

47. An isolated or recombinant 3-deoxy-D-glycero-pentanoic acid biosynthetic enzyme system that comprises: (A) a D-xylonic acid dehydratase, and (B) a 2-keto-acid reductase, the enzyme system being capable of catalyzing the conversion of D-xylonate to 3-deoxy-D-glycero-pentanoic acid.

48. An isolated or recombinant D-3,4-dihydroxy-butanoic acid biosynthetic enzyme system that comprises: (A) a D-xylose dehydrogenase, (B) a D-xylonic acid dehydratase, and (C) a 2-keto-acid decarboxylase, and (D) an aldehyde dehydrogenase, the enzyme system being capable of catalyzing the conversion of D-xylose to D-3,4-dihydroxy-butanoic acid.

49. An isolated or recombinant D-3,4-dihydroxy-butanoic acid biosynthetic enzyme system that comprises: (A) a D-xylonic acid dehydratase, (B) a 2-keto-acid decarboxylase, and (C) an aldehyde dehydrogenase, the enzyme system being capable of catalyzing the conversion of D-xylonate to D-3,4-dihydroxy-butanoic acid.

50. An isolated or recombinant (4S)-2-amino-4,5-dihydroxy pentanoic acid biosynthetic enzyme system that comprises: (A) a D-xylose dehydrogenase, (B) a D-xylonic acid dehydratase, and (C) a 2-keto acid transaminase, the enzyme system being capable of catalyzing the conversion of D-xylose to (4S)-2-amino-4,5-dihydroxy pentanoic acid.

51. An isolated or recombinant (4S)-2-amino-4,5-dihydroxy pentanoic acid biosynthetic enzyme system that comprises: (A) a D-xylonic acid dehydratase, and (B) a 2-keto acid transaminase, the enzyme system being capable of catalyzing the conversion of D-xylonate to (4S)-2-amino-4,5-dihydroxy pentanoic acid.

52-54. (canceled)

55. A process for screening for candidate enzyme-encoding polynucleotides, comprising:
(A) providing
(1) a nucleic acid or nucleic acid analog probe comprising a nucleobase sequence identical to that of about 20 or more contiguous nucleotides of a coding sequence that encodes an enzyme polypeptide having any one of
(a) the amino acid sequence of any one of SEQ ID NOs:2, 4, 6, 8, 10, 12, or 14, or
(b) the amino acid sequence of residues 19-319 of SEQ ID NO:12, or
(c) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or
(d) the amino acid sequence of a biocatalytic activity retaining conservative substituted variant of or homologous amino acid sequence to any of (a), (b), or (c); and
(2) a test sample comprising or suspected of comprising at least one target nucleic polynucleotide to which such a probe can specifically bind;
(B) contacting the probe with the test sample under conditions in which the probe can specifically hybridize to a target polynucleotide, if present, to form a probe-target polynucleotide complex, and
(C) detecting whether or not any probe-target polynucleotide complexes were formed thereby,
wherein a target polynucleotide that was identified as part of a complex is thereby identified as a candidate enzyme-encoding polynucleotide.

56. An antibody having specificity for an epitope of:
(A) an enzyme polypeptide having any one of
(1) the amino acid sequence of any one of SEQ ID NOs:2, 4, 6, 8, 10, 12, or 14, or
(2) the amino acid sequence of residues 19-319 of SEQ ID NO:12, or
(3) the amino acid sequence of a Pseudomonas fragi (ATCC 4973) D-xylonate dehydratase that comprises a polypeptide having a putative length of about 430+ residues, an approximate MW of about 60 kDa, and a C-terminal portion comprising the amino acid sequence of SEQ ID NO:10 near its end, or
(4) the amino acid sequence of a biocatalytic activity-retaining conservative substituted variant of or homologous amino acid sequence to any of (1), (2), or (3); or
(B) a polynucleotide or nucleic acid analog having a base sequence encoding such an enzyme polypeptide (A).

57. (canceled)
